(19) United States
(12) Patent Application Publication
(10) Pub. No.: US 2025/0327070 A1
(43) Pub. Date: Oct. 23, 2025

(54) COMPOSITIONS AND METHODS FOR INHIBITING COMPLEMENT FACTOR B

(71) Applicant: ASTRAZENECA IRELAND LIMITED, Dublin (IE)

(72) Inventors: Melissa Lasaro, Sandy Hook, CT (US); Susan Faas McKnight, Old Lyme, CT (US); Jihye Park, Newtonville, MA (US); Bob Dale Brown, Littleton, MA (US); Chengjung Lai, Hudson, NH (US); Henryk T. Dudek, Woburn, MA (US); Kathleen Nicole Beasley, Boston, MA (US); SungKwon Kim, Cheshire, CT (US)

(73) Assignee: ASTRAZENECA IRELAND LIMITED, Dublin (IE)

(21) Appl. No.: 18/292,187

(22) PCT Filed: Jan. 20, 2023
(86) PCT No.: PCT/US2023/011200

§ 371 Date: Mar. 10, 2024

Related U.S. Application Data
(60) Provisional application No. 63/301,454, filed on Jan. 20, 2022.
(60) Provisional application No. 63/328,629, filed on Apr. 7, 2022.

(51) Int. Cl. C12N 15/113; A61P 13/12

(52) U.S. Cl. CPC C12N 15/113; A61P 13/12; C12N 2310/14; C12N 2310/321; C12N 2310/322; C12N 2310/351; C12N 2310/531

(57) ABSTRACT

Described herein are oligonucleotides (e.g., RNAi oligonucleotides) containing sense and antisense strands for targeting complement factor B (CFB) mRNA. The RNAi oligonucleotide may be used to inhibit CFB expression, levels, and/or activity in a cell. Also, described herein are methods for using an oligonucleotide (e.g., an RNAi oligonucleotide) for the prophylaxis or treatment of a disease, disorder, or condition mediated by complement pathway activation or dysregulation.

### SEQUENCE LISTING

The present application is being filed along with a Sequence Listing in electronic format. The Sequence Listing is provided as a file entitled 50694-092WO3_Sequence_Listing_1_19_23_ST26.xml created on Jan. 19, 2023, which is 394.2 kilobytes in size. The information in electronic format of the sequence listing is incorporated herein by reference in its entirety.

### BACKGROUND

The complement system plays a central role in the clearance of immune complexes and in immune responses to infectious agents, foreign antigens, virus-infected cells, and tumor cells. Complement consists of a group of more than 50 proteins that form part of the innate immune system. The complement system is poised to defend the body from microbial infections and functions to maintain tissue hemostasis. Complement is a tightly regulated enzymatic cascade that can be activated by one of three pathways: the classical pathway, in which antibody complexes trigger activation, the alternative pathway, which is constitutively activated at a low level by a process called “tickover”, and which can be amplified by bacterial pathogens or injured tissue surfaces, and the lectin pathway, which is initiated by mannose residues found on certain microorganisms including certain bacteria, fungi, and viruses. Uncontrolled activation or insufficient regulation of the complement pathway can lead to systemic inflammation, cellular injury, and tissue damage. Thus, the complement pathway has been implicated in the pathogenesis of a number of diverse diseases. Inhibition or modulation of complement pathway activity has been recognized as a promising therapeutic strategy. The number of treatment options available for these diseases is limited. Thus, developing innovative strategies to treat diseases associated with complement pathway activation or dysregulation is a significant unmet need.

Complement factor B (CFB) is a component of the complement pathway that initiates the alternative complement pathway cascade. CFB is cleaved into Ba and Bb fragments. The Bb fragment associates with C3b and together they form the C3 convertase, which is integral to activation of the alternative complement pathway. Dysregulation or excessive activation of CFB has been linked to several diseases, including paroxysmal nocturnal hemoglobinuria (PNH), multiple sclerosis, and rheumatoid arthritis.

There exists a need for compositions and methods that can be used to inhibit or silence CFB in a subject with a disease associated with complement pathway activation or dysregulation.

### SUMMARY OF THE DISCLOSURE

Described herein are oligonucleotides (e.g., RNAi oligonucleotides, including sense and antisense strand oligonucleotides) that target complement factor B (CFB), which is known to play a role in complement pathway activation. The RNAi oligonucleotides, or a pharmaceutically acceptable salt thereof (e.g., a sodium salt thereof), may be used to treat patients with diseases associated with complement pathway activation or dysregulation.

A first aspect of the disclosure provides RNAi oligonucleotides, or a pharmaceutically acceptable salt thereof (e.g., a sodium salt thereof), for reducing complement factor B (CFB) expression, in which the oligonucleotide includes a sense strand and an antisense strand. The sense strand and the antisense strand of the oligonucleotide form a duplex region. The antisense strand of the oligonucleotide includes a region of complementarity to a CFB mRNA target sequence of, for example, SEQ ID NO: 13 or 14, and the region of complementarity is at least 15 contiguous nucleotides in length (e.g., at least 16, 17, 18, 19, 20, 21, 22, 23, 24, 25, 26, 27, 28, 29, or 30 contiguous nucleotides in length). In an embodiment, the sense strand is 15 to 50 nucleotides in length (e.g., 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length). In an embodiment, the sense strand is 18 to 36 nucleotides in length (e.g., 19, 20, 21, 22, 23, 24, 25, 26, 27, 28, 29, 30, 31, 32, 33, 34, 35, or 36 nucleotides in length). In an embodiment, the antisense strand is 15 to 30 nucleotides in length (e.g., 16, 17, 18, 19, 20, 21, 22, 23, 24, 25, 26, 27, 28, 29, or 30 nucleotides in length).

In an embodiment, the antisense strand of the oligonucleotide is 22 nucleotides in length, and the antisense strand and the sense strand form a duplex region of at least 19 nucleotides in length, optionally at least 20 nucleotides in length. In some embodiments, the sense strand is 36 nucleotides in length, and the antisense strand and the sense strand form a duplex region of at least 19 nucleotides in length, optionally at least 20 nucleotides in length. In some embodiments, the region of complementarity is at least 19 contiguous nucleotides in length, optionally at least 20 nucleotides in length.

In some embodiments, the 3′ end of the sense strand of the oligonucleotide includes a stem-loop set forth as S1-L-S2, in which S1 is complementary to S2, and in which L forms a loop between S1 and S2 that is 3-5 nucleotides (e.g., 3, 4, or 5 nucleotides) in length. In some embodiments, L is a triloop or a tetraloop. In an embodiment, L is a tetraloop. In an embodiment, the tetraloop includes the nucleic acid sequence of 5′ GAAA 3′.

In some embodiments, S1 and S2 of the stem-loop are 1-10 nucleotides in length (e.g., 2, 3, 4, 5, 6, 7, 8, 9, or 10 nucleotides in length). S1 and S2 may have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In an embodiment, S1 and S2 are 6 nucleotides in length.

In some embodiments, the stem-loop region includes a nucleic acid sequence with at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 7. In some embodiments, the stem-loop region includes a nucleic acid sequence with at least 95% (e.g., at least 96%, 97%, 98%, 99%, or 100%) sequence identity to the nucleic acid sequence of SEQ ID NO: 7. In some embodiments, the stem-loop includes the sequence 5′-GCAGCCGAAAGGCUGC-3′ (SEQ ID NO: 7). In some embodiments, the stem-loop includes a nucleic acid with up to 1, 2, or 3 nucleic acid substitutions, insertions, or deletions relative to the sequence of SEQ ID NO: 7.

In some embodiments, the antisense strand of the oligonucleotide includes a 3′ overhang sequence of one or more nucleotides in length. In some embodiments, the antisense strand includes a 3′ overhang of at least 2 linked nucleotides. In an embodiment, the 3′ overhang sequence is 2 nucleotides in length, such as a sequence is GG. In some embodiments, the sense strand includes a 5′ overhang of at least 2 linked nucleotides.

In some embodiments, the oligonucleotide includes at least one modified nucleotide. In some embodiments, the oligonucleotide includes between 20 and 50 modified nucleotides (e.g., 21, 22, 23, 24, 25, 26, 27, 28, 29, 30, 31, 32, 33, 34, 35, 36, 37. 38, 39, 40, 41, 42, 43, 44, 45, 46, 47, 48, 49, or 50 modified nucleotides). In some embodiments, the oligonucleotide includes between 20 and 40 (e.g., between 25 and 40, 30 and 40, 35 and 40, 30 and 35, 25 and 35, 20 and 25, 21 and 30, and 31 and 40) modified nucleotides. In an embodiment, all of the nucleotides of the oligonucleotide are modified.

The modified nucleotide may contain a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the 2′-modification is a 2′-fluoro or 2′-O-methyl, in which, optionally, the 2′-fluoro modification is 2′-fluoro deoxyribonucleoside and/or the 2′-O-methyl modification is 2′-O-methyl ribonucleoside. In some embodiments, the RNAi oligonucleotide, or pharmaceutically acceptable salt thereof, includes between 40 and 50 (e.g., 41, 42, 43, 44, 45, 46, 47, 48, 49, or 50) 2′-O-methyl modifications, in which, optionally, the RNAi oligonucleotide, or pharmaceutically acceptable salt thereof, includes between 40 and 50 (e.g., 41, 42, 43, 44, 45, 46, 47, 48, 49, or 50) 2′-O-methyl ribonucleosides.

In some embodiments, at least one of nucleotides 1-7, 12-27, and 31-36 of the sense strand and at least one of nucleotides 1, 4, 6, 8, 9, 11-13, and 15-22 of the antisense strand are modified with a 2′-O-methyl, such as a 2′-O-methyl ribonucleoside. In some embodiments, between 10 and 29 (e.g., 11, 12, 13, 14, 15, 16, 17, 18, 19, 20, 21, 22, 23, 24, 25, 26, 27, 28, or 29) of nucleotides 1-7, 12-27, and 31-36 of the sense strand and between 10 and 16 (e.g., 11, 12, 13, 14, 15, or 16) of nucleotides 1, 4, 6, 8, 9, 11-13, and 15-22 of the antisense strand are modified with a 2′-O-methyl, such as a 2′-O-methyl ribonucleoside. In an embodiment, all of nucleotides 1-7, 12-27, and 31-36 of the sense strand and all of nucleotides 1, 4, 6, 8, 9, 11-13, and 15-22 of the antisense strand are modified with a 2′-O-methyl, such as a 2′-O-methyl ribonucleoside. In an embodiment, all of nucleotides 1-7, 12-27, and 31-36 of the sense strand and all of nucleotides 1, 6, 8, 9, 11-13, and 15-22 of the antisense strand are modified with a 2′-O-methyl, such as a 2′-O-methyl ribonucleoside.

In some embodiments, the RNAi oligonucleotide, or pharmaceutically acceptable salt thereof, includes between 5 and 15 (e.g., 6, 7, 8, 9, 10, 11, 12, 13, 14, or 15) 2′-fluoro modified nucleotides, such as 2′-fluoro deoxyribonucleosides. In some embodiments, at least one of nucleotides 8, 9, 10, and 11 of the sense strand and at least one of nucleotides 2, 3, 4, 5, 7, 10 and 14 of the antisense strand are modified with a 2′-fluoro modified nucleotide, such as 2′-fluoro deoxyribonucleoside. In some embodiments, between 2 and 4 (e.g., 2, 3, and 4) of nucleotides 8, 9, 10, and 11 of the sense strand and between 2 and 7 (e.g., 2, 3, 4, 5, 6, and 7) of nucleotides 2, 3, 4, 5, 7, 10 and 14 of the antisense strand are modified with a 2′-fluoro modified nucleotide, such as 2′-fluoro deoxyribonucleoside. In an embodiment, all of nucleotides 8, 9, 10, and 11 of the sense strand and all of nucleotides 2, 3, 5, 7, 10 and 14 of the antisense strand are modified with a 2′-fluoro modified nucleotide, such as 2′-fluoro deoxyribonucleoside. In an embodiment, all of nucleotides 8, 9, 10, and 11 of the sense strand and all of nucleotides 2, 3, 4, 5, 7, 10, and 14 of the antisense strand are modified with a 2′-fluoro modified nucleotide, such as 2′-fluoro deoxyribonucleoside.

In an embodiment, the sense strand has a nucleic acid sequence of SEQ ID NO: 37 and the antisense strand has a nucleic acid sequence of SEQ ID NO: 38. In an embodiment, the sense strand has a nucleic acid sequence of SEQ ID NO: 66 and the antisense strand has a nucleic acid sequence of SEQ ID NO: 67.

In some embodiments, the RNAi oligonucleotide, or pharmaceutically acceptable salt thereof, includes at least one modified internucleotide linkage. In an embodiment, the at least one modified internucleotide linkage is a phosphorothioate linkage. In some embodiments, the RNAi oligonucleotide, or pharmaceutically acceptable salt thereof, has a phosphorothioate linkage between nucleotides 1 and 2 of the sense strand and nucleotides 1 and 2, 2 and 3, 20 and 21, and 21 and 22 of the antisense strand. In some embodiments, there is no internucleotide linkage between the sense strand and the antisense strand.

In some embodiments, the 4′-carbon of the sugar of the 5′-nucleotide of the antisense strand includes a phosphate analog. In some embodiments, the RNAi oligonucleotide, or pharmaceutically acceptable salt thereof, includes a uridine at the first position of the 5′ end of the antisense strand. In an embodiment, the uridine includes a phosphate analog. In an embodiment, the phosphate analog is 4′-O-monomethyl phosphonate. In embodiment, the uridine including the phosphate analog includes the following structure:

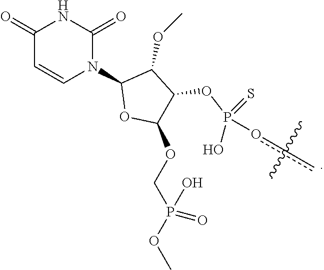

In some embodiments, at least one nucleotide of the oligonucleotide is conjugated to one or more targeting ligands. In some embodiments, each targeting ligand includes a carbohydrate, amino sugar, cholesterol, polypeptide, or lipid. In some embodiments, each targeting ligand includes an N-acetylgalactosamine (GalNAc) moiety. In some embodiments, the GalNAc moiety is a monovalent GalNAc moiety, a bivalent GalNAc moiety, a trivalent GalNAc moiety or a tetravalent GalNAc moiety. In some embodiments, the RNAi oligonucleotide includes between one and five (e.g., 1, 2, 3, 4, and 5) 2′-O—N-acetylgalactosamine (GalNAc) moieties conjugated to the sense strand. In an embodiment, up to 4 nucleotides of L of the stem-loop are conjugated to a monovalent GalNAc moiety. In an embodiment, 3 nucleotides of L of the stem-loop are conjugated to a monovalent GalNAc moiety. In some embodiments, one or more of the nucleotides at nucleotides positions 28-30 on the sense strand is conjugated to a monovalent GalNAc moiety. In an embodiment, each of the nucleotides at positions 28-30 of any one of SEQ ID NOs: 1, 4, 17, 19, 21, 23, 25, 27, and 29 (and variants thereof with at least 85% sequence identity thereto) is conjugated to a monovalent GalNAc moiety. In an embodiment, the nucleotides at positions 28-30 of any one of SEQ ID NOs: 1, 4, 17, 19, 21, 23, 25, 27, and 29 (and variants thereof with at least 85% sequence identity thereto) include the structure:

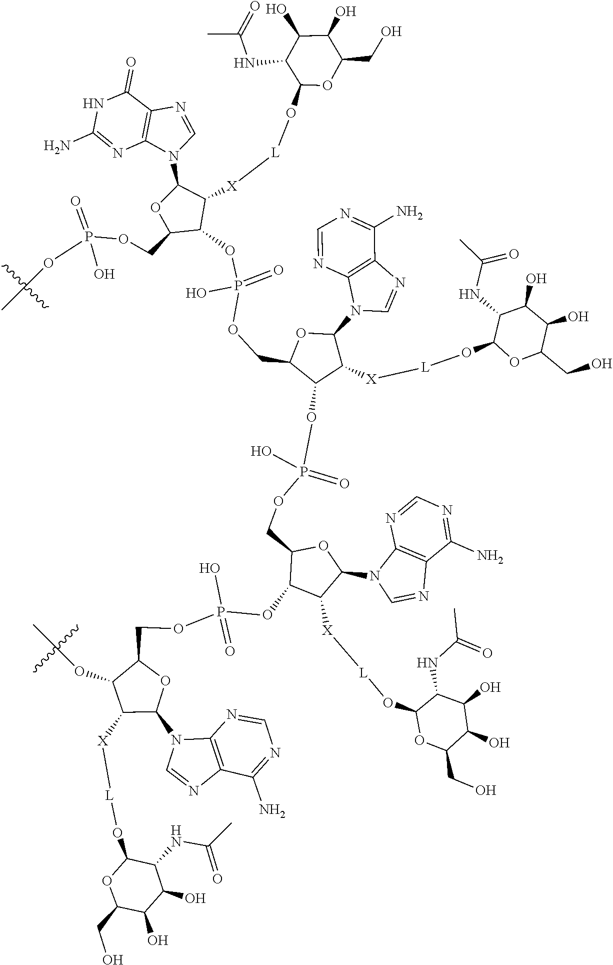

in which Z represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is an O, S, or N. In some embodiments, Z is an acetal linker. In some embodiments, X is O. In an embodiment, the nucleotides at positions 28-30 of any one of SEQ ID NOs: 1, 4, 17, 19, 21, 23, 25, 27, and 29 (and variants thereof with at least 85% sequence identity thereto) include the structure:

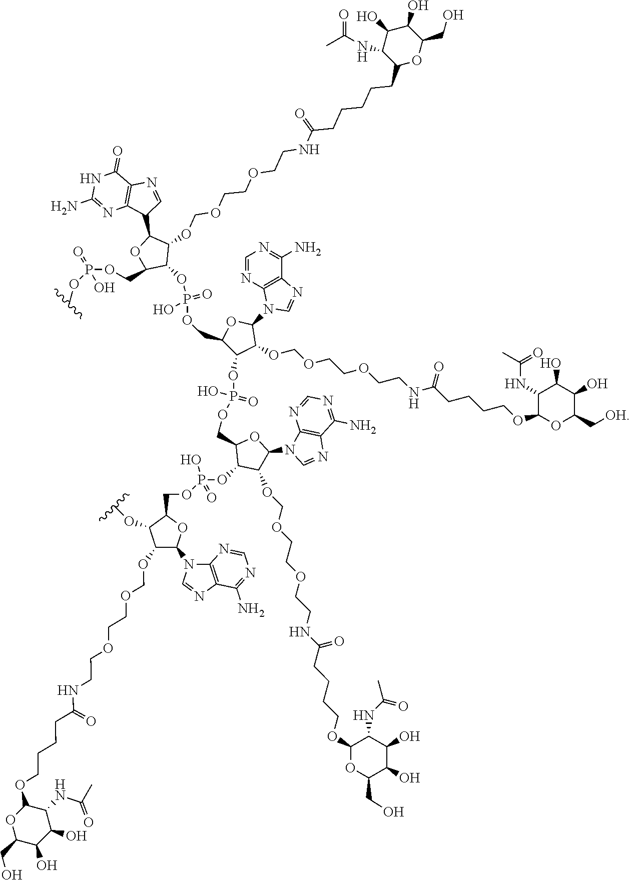

In some embodiments, an RNAi oligonucleotide herein, or pharmaceutically acceptable salt thereof, comprises a sense strand having a tetraloop, wherein three (3) GalNAc moieties are conjugated to nucleotides comprising the tetraloop, and wherein each GalNAc moiety is conjugated to one (1) nucleotide. In some embodiments, an oligonucleotide herein (e.g., an RNAi oligonucleotide) comprises a sense strand having a tetraloop comprising GalNAc-conjugated nucleotides, wherein the tetraloop comprises the following structure:

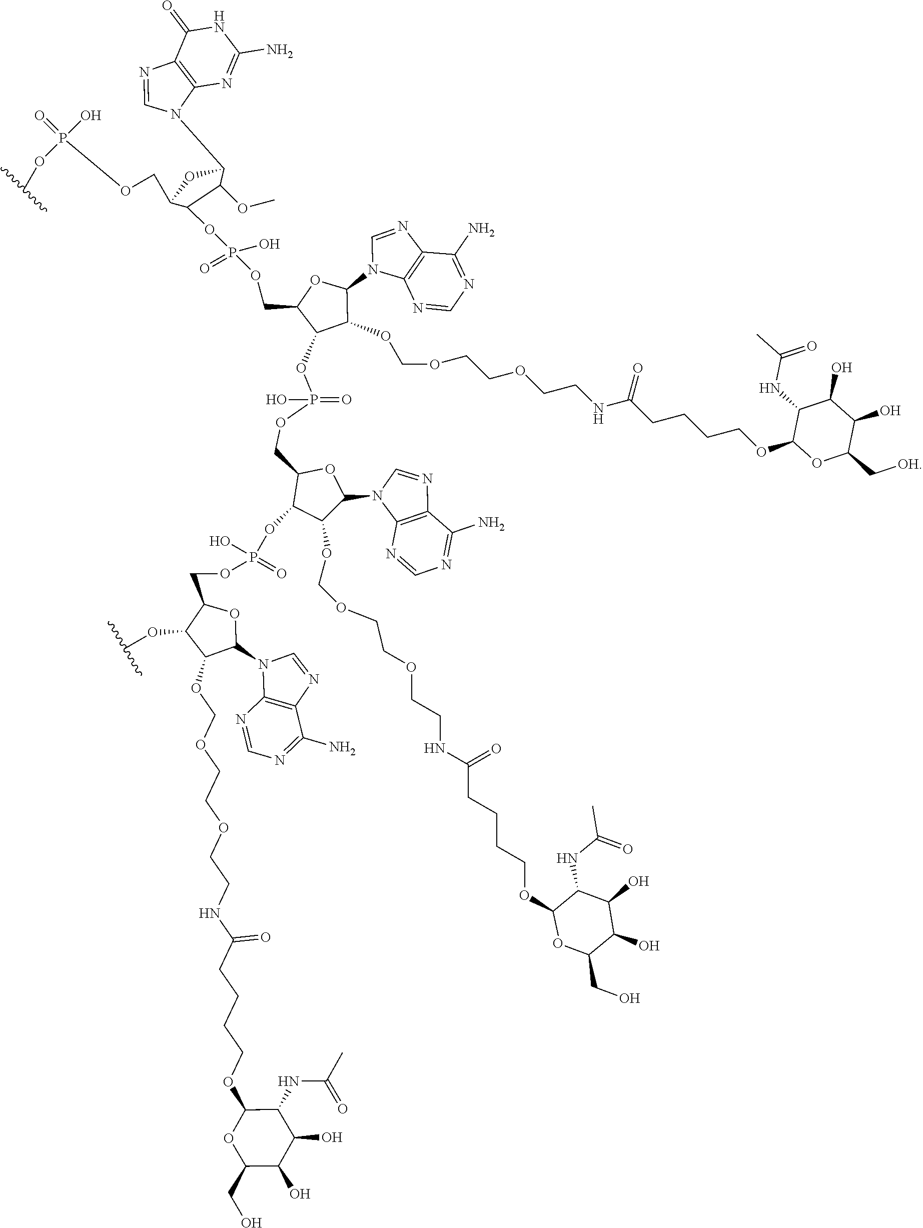

In some embodiments, the sense strand of the RNAi oligonucleotide, or pharmaceutically acceptable salt thereof, includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 1, SEQ ID NO: 2, SEQ ID NO: 4., or SEQ ID NO: 5. In some embodiments, the antisense strand of the oligonucleotide (e.g., an RNAi oligonucleotide) includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 3 or SEQ ID NO: 6. In some embodiments, the sense strand includes a nucleotide sequence having at least 95% (e.g., at least 96%, 97%, 98%, 99%, or 100%) sequence identity to SEQ ID NO: 1, SEQ ID NO: 2, SEQ ID NO: 4, or SEQ ID NO: 5. In some embodiments, the antisense strand includes a nucleotide sequence having at least 95% (e.g., at least 95%, 96%, 97%, 98%, 99%, or 100%) sequence identity to SEQ ID NO: 3 or SEQ ID NO: 6. In some embodiments, the sense strand includes a nucleotide sequence of SEQ ID NO: 1 or SEQ ID NO: 4. In some embodiments, the antisense strand includes a nucleotide sequence of SEQ ID NO: 3 or SEQ ID NO: 6. In some embodiments, the sense strand and antisense strands include nucleotide sequences selected from the group consisting of SEQ ID NOs: 1 and 3, respectively, and SEQ ID NOs: 4 and 6, respectively.

In an embodiment, the sense strand includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 1 and the antisense strand includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 3. In another embodiment, the sense strand includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 4 and the antisense strand includes a nucleotide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 6. In an embodiment, the sense strand includes a nucleotide sequence having at least 95% (e.g., at least 95%, 96%, 97%, 98%, 99%, or 100%) sequence identity to SEQ ID NO: 1 and the antisense strand includes a nucleotide sequence having at least 95% (e.g., at least 95%, 96%, 97%, 98%, 99%, or 100%) sequence identity to SEQ ID NO: 3. In another embodiment, the sense strand comprises a nucleotide sequence having at least 95% (e.g., at least 95%, 96%, 97%, 98%, 99%, or 100%) sequence identity to SEQ ID NO: 4 and the antisense strand comprises a nucleotide sequence having at least 95% (e.g., at least 95%, 96%, 97%, 98%, 99%, or 100%) sequence identity to SEQ ID NO: 6. In another embodiment, the sense strand comprises a nucleotide sequence as set forth in SEQ ID NO: 1 and the antisense strand comprises a nucleotide sequence as set forth in SEQ ID NO: 3. In another embodiment, the sense strand comprises a nucleotide sequence as set forth in SEQ ID NO: 4 and the antisense strand comprises a nucleotide sequence as set forth in SEQ ID NO: 6. In an embodiment, the sense strand has a nucleic acid sequence of SEQ ID NO: 37 and the antisense strand has the a nucleic acid sequence of SEQ ID NO: 38. In an embodiment, the sense strand has a nucleic acid sequence of SEQ ID NO: 66 and the antisense strand has the a nucleic acid sequence of SEQ ID NO: 67.

In some embodiments, the RNA oligonucleotide includes a pharmaceutically acceptable salt. In some embodiments, the pharmaceutically acceptable salt is a sodium salt.

In a second aspect, the disclosure provides a pharmaceutical composition comprising any one of the RNA oligonucleotides, or pharmaceutically acceptable salt thereof, described herein and a pharmaceutically acceptable carrier, excipient, or diluent.

In a third aspect, the disclosure provides a vector encoding one or both of the sense and antisense strands of the RNAi oligonucleotides, or pharmaceutically acceptable salt thereof, described herein.

In a fourth aspect, the disclosure provides a cell comprising the vector encoding all or a part of the RNAi oligonucleotides, or pharmaceutically acceptable salt thereof, described herein.

In a fifth aspect, the disclosure provides a method for treating a subject with a disease, disorder, or condition associated with complement pathway activation or dysregulation (e.g., activation or dysregulation of CFB) comprising administering to the subject a therapeutically effective amount of any one or more of the RNAi oligonucleotides, or pharmaceutically acceptable salt thereof, described herein or a pharmaceutical composition containing the same, a vector encoding the same, or a cell containing the oligonucleotide(s) or vector(s), as described herein. In some embodiments, the RNAi oligonucleotide degrades an mRNA transcript of CFB in a cell of the subject. In some embodiments, the expression of CFB in a cell of the subject is reduced. In some embodiments, the expression of CFB in a cell of the subject is reduced by between 10% and 100% (e.g., between 10% and 90%, 10% and 70%, 10% and 50%, 10% and 30%, 20% and 100%, 40% and 100%, 60% and 100%, and 80% and 100%) relative to the level of expression of CFB in a cell of a subject that is not administered an RNAi oligonucleotide, pharmaceutical composition, vector, or cell described herein. In some embodiments, the level and/or activity of CFB in the subject is reduced. In some embodiments, the level and/or activity of CFB is reduced by between 10% and 100% (e.g., between 10% and 90%, 10% and 70%, 10% and 50%, 10% and 30%, 20% and 100%, 40% and 100%, 60% and 100%, and 80% and 100%) relative to the level and/or activity of CFB in a subject that is not administered an RNAi oligonucleotide, pharmaceutical composition, vector, or cell described herein. In some embodiments, the level and/or activity of CFB is reduced by between 50% and 100% relative (e.g., between 50% and 90%, 50% and 80%, 50% and 70%, 50% and 60%, 60% and 100%, 70% and 100%, 80% and 100%, and 90% and 100%) to the level and/or activity of CFB in a subject that is not administered an RNAi oligonucleotide, pharmaceutical composition, vector, or cell described herein. In some embodiments, administration of an RNAi oligonucleotide, pharmaceutical composition, vector, or cell, as described herein, to a subject in need thereof reduces the amount of CFB circulating in the blood of the subject, relative to a subject that is not administered an RNAi oligonucleotide, pharmaceutical composition, vector, or cell described herein (an untreated subject). The amount of CFB in the blood of a treated subject may be reduced to less than 1,000 μg/mL, 900 μg/mL, 800 μg/mL, 700 μg/mL, 600 μg/mL, 500 μg/mL, 400 μg/mL, 300 μg/mL, 200 μg/mL, 100 μg/mL, or 50 μg/mL, or less. For example, administration of an RNAi oligonucleotide, pharmaceutical composition, vector, or cell, as described herein, may reduce the amount of CFB in the blood of a treated subject to within the range of 50-1000 μg/mL (e.g., within the range of 50-900 μg/mL, 50-800 μg/mL, 50-700 μg/mL, 50-600 μg/mL, 50-500 μg/mL, 50-400 μg/mL, 50-300 μg/mL, or 50-200 μg/mL) or to less than 50 μg/mL.

In an embodiment of the fifth aspect, the subject is a mammal, such as a human.

In some embodiments, the RNAi oligonucleotide, or pharmaceutically acceptable salt thereof, the pharmaceutical composition, the vector, or the cell is formulated for daily, weekly, monthly, or yearly administration. In some embodiments, the RNAi oligonucleotide, or pharmaceutically acceptable salt thereof, the pharmaceutical composition, the vector, or the cell is formulated for intravenous, subcutaneous, intramuscular, oral, nasal, sublingual, intrathecal, and intradermal administration. In an embodiment, the RNAi oligonucleotide, or pharmaceutically acceptable salt thereof, the pharmaceutical composition, the vector, or the cell is formulated for subcutaneous administration. In an embodiment, the RNAi oligonucleotide, or pharmaceutically acceptable salt thereof, the pharmaceutical composition, the vector, or the cell is formulated for administration at a dosage of between about 0.1 mg/kg to about 150 mg/kg (e.g., 0.1 mg/kg to 100 mg/kg, 0.1 mg/kg to 50 mg/kg, 0.1 mg/kg to 1 mg/kg, 1 mg/kg to 150 mg/kg, 50 mg/kg to 150 mg/kg, and 100 mg/kg to 150 mg/kg).

In a sixth aspect, the disclosure provides a method for reducing CFB expression in a cell, a population of cells, or a subject by contacting the cell, the population of cells, or the subject with an oligonucleotide(s) (e.g., an RNAi oligonucleotide) of the disclosure, or a pharmaceutical composition containing the oligonucleotide(s), a vector encoding the oligonucleotide(s), or a cell containing the vector, as described herein. The subject can be administered an oligonucleotide(s) (e.g., an RNAi oligonucleotide(s)) of the disclosure, or a pharmaceutical composition containing the oligonucleotide(s), a vector encoding the oligonucleotides, or a cell containing the vector, as described herein. In some embodiments, reducing CFB expression comprises reducing an amount or level of CFB mRNA, an amount or level of CFB protein, or both. In some embodiments, the level of CFB mRNA, level of CFB protein, or both is reduced by between 10% and 100% (e.g., between 10% and 80%, 10% and 60%, 10% and 40%, 10% and 20%, 20% and 100%, 40% and 100%, 60% and 100%, and 80% and 100%) relative to the level of CFB mRNA, level of CFB protein, or both in the cell of a subject that is not administered the oligonucleotide(s) (e.g., the RNAi oligonucleotide(s)), the pharmaceutical composition, the vector, or the cell, as described herein. In some embodiments, the level of CFB mRNA, level of CFB protein, or both is reduced by between 50% and 100% (e.g., between 50% and 90%, 50% and 80%, 50% and 70%, 50% and 60%, 60% and 100%, 70% and 100%, 80% and 100%, and 90% and 100%) relative to the level of CFB mRNA, level of CFB protein, or both in the cell of a subject that is not administered the RNAi oligonucleotide, the pharmaceutical composition, the vector, or the cell, as described herein. In some embodiments, administration of an RNAi oligonucleotide, pharmaceutical composition, vector, or cell, as described herein, to a subject in need thereof reduces the amount of CFB circulating in the blood of the subject, relative to a subject that is not administered an RNAi oligonucleotide, pharmaceutical composition, vector, or cell described herein (an untreated subject). The amount of CFB in the blood of a treated subject may be reduced to less than 1,000 μg/mL, 900 μg/mL, 800 μg/mL, 700 μg/mL, 600 μg/mL, 500 μg/mL, 400 μg/mL, 300 μg/mL, 200 g/mL, 100 g/mL, or 50 μg/mL, or less. For example, administration of an RNAi oligonucleotide, pharmaceutical composition, vector, or cell, as described herein, may reduce the amount of CFB in the blood of a treated subject to within the range of 50-1000 μg/mL (e.g., within the range of 50-900 μg/mL, 50-800 μg/mL, 50-700 μg/mL, 50-600 μg/mL, 50-500 g/mL, 50-400 μg/mL, 50-300 g/mL, or 50-200 μg/mL) or to less than 50 μg/mL.

In a seventh aspect, the disclosure provides a kit comprising an oligonucleotide(s) (e.g., an RNAi oligonucleotide(s)), a pharmaceutical composition, a vector, or a cell, as described herein. In some embodiments, the kit includes a pharmaceutical composition which includes an RNAi oligonucleotide, or pharmaceutically acceptable salt thereof, agent that reduces the level and/or activity of CFB in a cell or subject described herein and, optionally, a pharmaceutically acceptable carrier, excipient, or diluent. In some embodiments, the kit includes a vector encoding any one of the RNAi oligonucleotides, pharmaceutical compositions, vectors, or cells described herein. In some embodiments, the kit includes a package insert with instructions to perform any of the methods described herein. In some embodiments, the kit includes a pharmaceutical composition including an RNAi oligonucleotide agent, pharmaceutical composition, vector, or cell described herein that reduces the level and/or activity of CFB in a cell or subject; an additional therapeutic agent; and a package insert with instructions to perform any of the methods described herein.

In an eighth aspect, the disclosure features the use of an oligonucleotide(s) (e.g., an RNAi oligonucleotide(s)), a pharmaceutical composition, a vector, or a cell, as described herein, for use in the prophylaxis or treatment of a disease, disorder, or condition mediated by complement pathway activation or dysregulation (e.g., CFB activation or dysregulation) in a subject in need thereof.

In any one of the fifth, sixth, or eighth aspects, the subject is identified as having a disease, disorder, or condition mediated by complement pathway activation or dysregulation (e.g., CFB activation of dysregulation). In some embodiments, the disease is paroxysmal nocturnal hemoglobinuria (PNH), C3 glomerulopathy (C3G), immunoglobulin A nephropathy (IgAN), membranous nephropathy (MN), including primary MN, E. coli-induced or typical hemolytic uremic syndrome (HUS), atypical hemolytic uremic syndrome (aHUS), age-related macular degeneration, geographic atrophy, diabetic retinopathy, uveitis, intermediate uveitis, Behcet's uveitis, retinitis pigmentosa, macular edema, multifocal choroiditis, Vogt-Koyanagi-Harada syndrome, birdshot retinochoriodopathy, sympathetic ophthalmia, ocular cicatricial pemphigoid (OCP), ocular pemphigus, nonarthritic ischemic optic neuropathy, post-operative inflammation, retinal vein occlusion, neurological disorders, multiple sclerosis, stroke, Guillain Barre Syndrome, traumatic brain injury, Parkinson's disease, hemodialysis complications, hyperacute allograft rejection, xenograft rejection, interleukin-2 (IL-2) induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, arthritis, autoimmune heart disease, inflammatory bowel disease, ischemia-reperfusion injuries, Barraquer-Simons Syndrome, hemodialysis, anti-neutrophil cytoplasmic antibody (ANCA) vasculitis, cryoglobulinemia, psoriasis, transplantation, diseases of the central nervous system such as Alzheimer's disease and other neurodegenerative conditions, dense deposit disease, blistering cutaneous diseases, membranoproliferative glomerulonephritis type II (MPGN II), chronic graft vs. host disease, Felty syndrome, pyoderma gangrenosum (PG), hidradenitis suppurativa (HS), pulmonary arterial hypertension, primary Sjogren's syndrome, primary biliary cholangitis, autosomal dominant polycystic kidney disease, and myelin oligodendrocyte glycoprotein antibody disease (MOGAD). In some embodiments, the disease is rheumatoid arthritis.

In some embodiments, the RNA oligonucleotide described herein includes a pharmaceutically acceptable salt. In some embodiments, the pharmaceutically acceptable salt is or includes acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate, methylamine, dimethylamine, trimethylamine, triethylamine, or ethylamine, or is an alkali or alkaline earth metal salt. In some embodiments, the alkali or alkaline earth metal salt is selected from the group consisting of sodium, lithium, potassium, calcium, magnesium, and ammonium (e.g., quaternary ammonium and tetramethylammonium). In some embodiments, the pharmaceutically acceptable salt is a sodium salt.

### BRIEF DESCRIPTION OF THE DRAWINGS

The patent or application file contains at least one drawing executed in color. Copies of this patent or patent application publication with color drawing(s) will be provided by the Office upon request of the payment of the necessary fee.

FIG. 1A shows the chemical structure of the sense strand of Compound A (SEQ ID NO: 66).

FIG. 1B shows the chemical structure of the antisense strand of Compound A (SEQ ID NO: 67).

FIG. 1C shows the nucleic acid sequence for the sense (SEQ ID NO: 1) and antisense (SEQ ID NO: 3) strands of Compound A.

FIG. 2A shows the chemical structure of the sense strand of Compound B (SEQ ID NO: 37).

FIG. 2B shows the chemical structure of the antisense strand of Compound B (SEQ ID NO: 38).

FIG. 2C shows the nucleic acid sequence for the sense (SEQ ID NO: 4) and antisense (SEQ ID NO: 6) strands of Compound B.

FIG. 2D shows a schematic drawing of the sense and antisense strands of Compound B.

FIG. 2E-1 and FIG. 2E-2 show the chemical structure of the RNAi oligonucleotide of Compound B (SEQ ID NOs: 37 and 38).

FIG. 3A is a graph showing the percentage of CFB mRNA remaining after HuH-7 cells were treated in vitro with various oligonucleotides in an amount of 1 nM. The effect of CFB knockdown promoted by Compounds A and B are specifically identified.

FIG. 3B is graph showing the percent of CFB mRNA remaining after treating HuH-7 cells in vitro with various oligonucleotides in an amount of 0.03 nM, 0.1 nM, and 1 nM. The effect of CFB knockdown promoted by Compound B is specifically identified.

FIG. 4A is a graph showing amount of CFB mRNA remaining in mice 4 days after subcutaneous administration of a single dose of 0.25 mg/kg, 0.5 mg/kg, 1.0 mg/kg, or 3.0 mg/kg of Compound A or Compound C.

FIG. 4B is a graph showing amount of CFB mRNA remaining in mice 4 days after subcutaneous administration of a single dose of 0.3 mg/kg of Compound A or Compound D.

FIG. 4C is a graph showing amount of CFB mRNA remaining in mice 4 days after subcutaneous administration of a single dose of 0.5 mg/kg of Compound A, B, E, F, G, H, or I.

FIG. 5 is a graph showing measurement of the percent CFB mRNA in the liver of cynomolgus macaques pre dose and 28 days and 56 days after treatment with a single dose of 4 mg/kg Compounds A-I as compared to PBS administered as a control.

FIG. 6A is a graph showing measurement of the percent of CFB mRNA in the liver of cynomolgus macaques after treatment with 1 mg/kg or 2 mg/kg Compound A or Compound B on days 0, 28, 56, and 84 as compared to PBS administered as a control.

FIG. 6B is a graph showing measurement of the percent of CFB in serum of cynomolgus macaques after treatment with 1 mg/kg or 2 mg/kg Compound A or Compound B as compared to PBS administered as a control.

FIG. 7 is a graph showing the approximate ED₅₀ for Compound A and Compound B measured as CFB mRNA in liver of cynomolgus macaques 28 days after a single dose of Compound A or Compound B at 2 mg/kg.

FIG. 8 is a graph showing the percent of complement activity (AP) in serum of cynomolgus macaques after treatment 2 mg/kg Compound A or Compound B on days 0, 28, 56 and 84 as measured by WIESLAB® ELISA-based functional assay. PBS was administered in the same multidose regimen as a control group.

FIG. 9 is a graph showing the percent of lysis from serum of cynomolgus macaques after treatment 1 mg/kg or 2 mg/kg Compound B on days 0, 28, 56 and 84 as measured by hemolysis of rabbit erythrocytes method. PBS was administered in the same multidose regimen as a control group.

FIG. 10A is a graph showing RT-qPCR measurement of the percent CFB mRNA in the liver of CD-1 mice after administration of a single, subcutaneous dose of 0.25 mg/kg, 0.5 mg/kg, and 3 mg/kg of Compound J as compared to PBS administered as a control. The levels of hepatic knockdown were followed for 63 days, and 5 mice were sacrificed at each time point for measurements.

FIG. 10B is a graph showing qualitative measurement by immunoblot of the percent CFB circulating protein in serum of CD-1 mice over a 42-day period after being administered a single, subcutaneous dose of Compound J of 0.25 mg/kg (second leftmost column), 0.5 mg/kg (third leftmost column), and 3 mg/kg (rightmost column) as compared to PBS administered as a control (leftmost column).

FIG. 11 is a graph showing stem loop-qPCR measurement of the amount of siRNA exposure in the plasma, spleen, liver, and kidney tissue of CD-1 mice administered a single, subcutaneous dose of 3 mg/kg of Compound J. The time-course of measurement was for a period of 672 hours. Five mice were sacrificed at each time point for measurements.

FIG. 12A is a graph showing RT-qPCR measurement of the percent of CFB mRNA in the liver of CD-1 mice over a period of 70 days following administration of 4 doses of 0.5 mg/kg (only 56 days) or 3 mg/kg Compound J on days 0, 14, 28, and 42 as compared to PBS administered as a control.

FIG. 12B is a graph showing qualitative measurement by immunoblot of the CFB serum protein in CD-1 mice over a period of 70 days following administration of 4 doses of 0.5 mg/kg or 3 mg/kg Compound J on days 0, 14, 28, and 42. CFB levels were calculated as a percentage to CFB serum levels measured from PBS control group (n=5/timepoint).

FIG. 13A is a graph showing stem loop-qPCR measurement of the concentration of Compound J in liver tissue of CD-1 mice dosed with 4 doses of 0.5 mg/kg Compound J on Days 0, 14, 28, and 42.

FIG. 13B is a graph showing stem loop-qPCR measurement of the concentration of Compound J in plasma of CD-1 mice dosed with 4 doses of 0.5 mg/kg Compound J on Days 0, 14, 28, and 42.

FIG. 14A is a graph showing the clinical score of the hind paws from collagen antibody-induced arthritis model in which arthritis was induced on Day 0 and an LPS booster on Day 3 and then prophylactically treated with 3 doses of 0.5 mg/kg or 3 mg/kg dose of Compound J on day −7, 0 and 7. PBS treated CAIA animals were used as control group.

FIG. 14B is a graph showing the clinical score of the hind paws from collagen antibody-induced arthritis model in which arthritis was induced on Day 0 and an LPS booster on Day 3 and then therapeutically treated with a single dose of 0.5 mg/kg or 3 mg/kg dose of Compound J on day 5 post disease induction. PBS-treated CAIA animals were used as control group.

FIG. 15A are images of hind paw inflammation on Day 11 of a CAIA mouse model in which arthritis was induced with a collagen antibody administered on Day 0 and an LPS booster on Day 3 and then prophylactically treated with 3 doses of 3 mg/kg dose of Compound J on day −7, 0 and 7. PBS treated CAIA animals were used as control group.

FIG. 15B are images of hind paw inflammation on Day 13 of a CAIA mouse model in which arthritis was induced with a collagen antibody administered on Day 0 and an LPS booster on Day 3 and then therapeutically treated with a single 3 mg/kg dose of Compound J on day 5 post disease induction. PBS treated CAIA animals were used as control group.

FIG. 16 are images of H&E staining demonstrating the reduction of mononuclear cells infiltration to the hind paws after prophylactic treatment with 3 doses of 3 mg/kg dose of Compound J on day −7, 0 and 7. Naïve and PBS-treated CAIA animals were used as negative and positive controls for inflammation, respectively.

FIG. 17 are images of Safranin O staining demonstrating prevention of cartilage erosion and pannus formation and H&E staining demonstrating reduction of mononuclear cell infiltration in the knee joint of CAIA-induced arthritis model after animals were prophylactically treated with 3 doses of 3 mg/kg Compound J on day −7, 0 and 7. Naïve and PBS treated CAIA animals were used as negative and positive controls, respectively.

FIG. 18 are images of Safranin O staining demonstrating prevention of cartilage erosion and pannus formation in the knee joint of CAIA-induced arthritis model after animals were prophylactically treated with 3 doses of 3 mg/kg Compound J on day −7, 0, and 7. Naïve and PBS treated CAIA animals were used as negative and positive controls, respectively.

FIG. 19 are images of lymphocyte (CD45+) staining of the hind paws of CAIA-induced arthritis animals demonstrating the reduction of immune cell infiltration after therapeutic treatment with a single dose of 3 mg/kg of Compound J on day 5 post disease induction. Naïve and PBS treated CAIA animals were used as negative and positive controls, respectively.

FIG. 20 are images of neutrophils and macrophages (CD11b+) staining of the hind paws of CAIA-induced arthritis animals demonstrating the reduction of immune cell infiltration after therapeutic treatment with a single dose of 3 mg/kg of Compound J on day 5 post disease induction. Naïve and PBS treated CAIA animals were used as negative and positive controls, respectively.

FIG. 21 are images of macrophage (F4/80+) staining of the hind paws of CAIA-induced arthritis animals demonstrating the reduction of immune cell infiltration after therapeutic treatment with a single dose of 3 mg/kg of Compound J on day 5 post disease induction. Naïve and PBS treated CAIA animals were used as negative and positive controls, respectively.

FIG. 22 shows images of in situ hybridization of fluorescent tags to CFB mRNA (red) to monitor local complement expression and CD45+ cells (green-lymphocytes) infiltration to the hind paw of CAIA-induced animals after therapeutic treatment with a single 3 mg/kg dose of Compound J on day 5 post disease induction.

FIG. 23 is a graph showing the mean clinical score from 2 experiments using MOG-induced experimental autoimmune encephalomyelitis (EAE) mice in which disease was induced on Day 0 and receive 2 doses of Pertussis toxin on Day 0 and 1 and then therapeutically treated with 5 weekly doses of 3 mg/kg dose of Compound J starting on day 7 post disease induction. PBS-treated EAE animals were used as disease positive control.

FIG. 24 shows representative images of Luxol fast blue spinal cord staining of MOG-induced EAE mice after 5 weekly doses of 3 mg/kg of Compound J in comparison to naïve, PBS-treated EAE mice were used as disease positive control.

FIG. 25A is a graph showing the amount of liver CFB mRNA in MOG-induced EAE mice after 5 weekly doses of 3 mg/kg of Compound J in comparison to naïve, PBS-treated EAE mice (disease positive control).

FIG. 25B is a graph showing the amount of serum CFB in MOG-induced EAE mice after 5 weekly doses of 3 mg/kg of Compound J in comparison to naïve, PBS-treated EAE mice (disease positive control).

FIG. 26 is a graph showing the ratio of proteinuria:creatinine measured from spot urine collection from Passive Heyman's nephritis (PHN) rat model in which proteinuria was induced on Day 0 with a single dose of sheep anti-Rat Fx1A and prophylactically treated with 3 doses of 12 mg/kg of Compound J on day −14, −7 and 0, in comparison to PBS-treated PHN animals (disease positive control) and healthy animals.

FIG. 27 is a graph showing the percent of lysis from serum of Passive Heyman's nephritis (PHN) rats after treatment with 12 mg/kg Compound J on days −14, −7, and 0 as measured by hemolysis of rabbit erythrocytes method before disease induction (day −1) or 6 days after disease induction, in comparison to PBS-treated PHN animals (disease positive control) and healthy animals.

### DEFINITIONS

As used herein, the terms “about” and “approximately” refer to an amount that is ±10% of the recited value and is optionally ±5% of the recited value, or more optionally ±2% of the recited value.

As used herein, “administering” and “administration” refers to any method of providing a pharmaceutical preparation to a subject. The oligonucleotides described herein may be administered by any method known to those skilled in the art. Suitable methods for administering an oligonucleotide may include, for example, orally, by injection (e.g., intravenously, intraperitoneally, intramuscularly, intravitreally, and subcutaneously), drop infusion preparations, and the like. Methods of administering an oligonucleotide may include subcutaneous administration. Oligonucleotides prepared as described herein may be administered in various forms, depending on the disorder to be treated and the age, condition, and body weight of the subject, as is known in the art. A preparation can be administered prophylactically; that is, administered to decrease the likelihood of developing a disease or condition.

As used herein, an “agent that reduces the level and/or activity of CFB” refers to an oligonucleotide (e.g., an RNAi oligonucleotide) disclosed herein that can be used (e.g., administered) to reduce the level or expression of CFB in a cell or subject, such as in the subject's cells or serum. By “reducing the level of CFB,” “reducing expression of CFB,” and “reducing transcription of CFB” is meant decreasing the level, decreasing the expression, or decreasing the transcription of CFB mRNA and/or CFB protein in a cell or subject, e.g., by administering an oligonucleotide agent (such as those described herein) to the cell or subject. The level of CFB mRNA and/or CFB protein may be measured using any method known in the art (e.g., by measuring the level of CFB mRNA or level of CFB protein in a cell or a subject). The reduction may be a decrease in the level, expression, or transcription of CFB mRNA and/or CFB protein of about 5% or more (e.g., about 10%, 20%, 30%, 40%, 50%, 60%, 70%, 80%, 90%, 95%, 96%, 97%, 98%, 99%, or about 100%) in a cell or subject compared to prior to treatment or relative to a level of CFB mRNA or CFB protein in an untreated subject (e.g., a subject with a disease or disorder associated with complement activation or dysregulation (e.g., activation or dysregulation of CFB) or relative to a control subject (e.g., a healthy subject (e.g., a subject without a disease or disorder associated with complement activation or dysregulation (e.g., activation or dysregulation of CFB)). The CFB may be any CFB (such as, e.g., mouse CFB, rat CFB, monkey CFB, or human CFB), as well as variants or mutants of CFB. Thus, the CFB may be a wild-type CFB, a mutant CFB, or a transgenic CFB in the context of a genetically manipulated cell, group of cells, or organism. “Reducing the activity of CFB” also means decreasing the level of an activity related to CFB (e.g., by reducing the activation of the complement pathway associated with a disease mediated by complement pathway activation or dysregulation). The activity of CFB may decreased by about 5% or more (e.g., about 10%, 20%, 30%, 40%, 50%, 60%, 70%, 80%, 90%, 95%, 96%, 97%, 98%, 99%, or about 100%). The activity level of CFB may be measured using any method known in the art. The reduction may be a decrease in the level, expression, or transcription of CFB mRNA and/or CFB protein of at least about 5% or more (e.g., about 10%, 20%, 30%, 40%, 50%, 60%, 70%, 80%, 90%, 95%, 96%, 97%, 98%, 99%, or about 100% or more, relative to a cell or a subject not treated with an oligonucleotide agent disclosed herein). This reduction in the level, expression, or transcription of CFB mRNA and/or CFB protein may be for a period of at least one day or more (e.g., at least 2 days, 3 days, 4 days, 5 days, 10 days, 15 days, 20 days, 30 days, 40 days, 50 days, 60 days, 70 days, 80 days, 90 days, 100 days, 110 days, 120 days, or more). The reduction may be a decrease in the amount of CFB protein in blood of a treated subject (e.g., a human subject) of at least 5 μg/mL (e.g., between at least 5-1000 μg/mL), such as for a period of at least 1 day (e.g., at least 2 days, 3 days, 4 days, 5 days, 10 days, 15 days, 20 days, 30 days, 40 days, 50 days, 60 days, 70 days, 80 days, or more).

The term “alternative nucleoside” or “alternative nucleotide” refers to a nucleoside having an alternative sugar or an alternative nucleobase, such as those described herein. An alternative nucleoside may include a nucleoside in which the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine, 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine. An alternative nucleoside may also include a nucleoside where the sugar moiety is modified; for example, 2′-O-methyladenosine, 2′-O-methylguanosine, 2′-O-methylcytosine, 2′-O-methyluridine, 2-fluoro-deoxyadenosine, 2-fluoro-deoxyguanosine, 2-fluoro-deoxycytidine, and 2-fluoro-deoxyuridine.

Exemplary nucleobases having an alternative uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (m⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

Exemplary nucleobases having an alternative cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

Exemplary nucleobases having an alternative adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

Exemplary nucleobases having an alternative guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, 2′-F-ara-guanosine, and 2′-F-guanosine.

The nucleobase moieties may be indicated by the letter code for each corresponding nucleobase, e.g., A, T, G, C, or U, wherein each letter may optionally include alternative nucleobases of equivalent function.

As used herein, the term “alternative complement pathway” refers to one of three pathways of complement activation, the others being the classical pathway and the lectin pathway.

The term “antisense,” as used herein, refers to an oligonucleotide that is sufficiently complementary to all or a portion of a gene, primary transcript, or processed mRNA (e.g., the sequence of CFB (e.g., SEQ ID NO: 12), so as to interfere with expression of the endogenous gene (e.g., CFB).

The terms “antisense strand” and “guide strand” refer to the strand of an RNAi oligonucleotide that includes a region that is substantially complementary to a target sequence, e.g., a CFB mRNA (e.g., SEQ ID NO: 12).

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 10 nucleotides of a 21-nucleotide nucleic acid molecule” means that a range of from 10-21 nucleotides, such as, e.g., 10, 11, 12, 13, 14, 15, 16, 17, 18, 19, 20, or 21 nucleotides, have the indicated property. When “at least” is present before a series of numbers or a range, it is understood that “at least” can modify each of the numbers in the series or range.

As used herein, the term “attenuates” means reduces or effectively halts. As a non-limiting example, one or more of the treatments provided herein may reduce or effectively halt the onset or progression of a disease mediated by complement pathway activation or dysregulation (e.g., CFB activation or dysregulation) in a subject. This attenuation may be exemplified by, for example, a decrease in one or more aspects (e.g., symptoms, tissue characteristics, and cellular, inflammatory or immunological activity, etc.) of a disease associated with complement pathway activation or dysregulation, such as for example, a disease described herein.

The term “cDNA” refers to a nucleic acid sequence that is a DNA equivalent of an mRNA sequence (i.e., having uridine substituted with thymidine). Generally, the terms cDNA and mRNA may be used interchangeably in reference to a particular gene (e.g., CFB gene) as one of skill in the art would understand that a cDNA sequence is the same as the mRNA sequence with the exception that uridines are read as thymidines.

As used herein the terms “CFB” and “complement factor B” refer to the protein or gene encoding complement factor B, depending upon the context in which the term is used. The term “CFB” also encompasses natural variants of the wild-type CFB protein, such as proteins having at least 85% sequence identity (e.g., at least 86%, 87%, 88%, 89%, 90%, 91%, 92%, 93%, 94%, 95%, 96%, 97%, 98%, 99%, or 99.9% sequence identity, or more) to the amino acid sequence of wild-type human CFB, which is set forth in NCBI Reference No: NP_001701.2 (SEQ ID NO: 11). The term “CFB” also refers to natural variants of the wild-type CFB gene, such as those having at least 85% identity (e.g., 85%, 86%, 87%, 88%, 89%, 90%, 91%, 92%, 93%, 94%, 95%, 96%, 97%, 98%, 99%, 99.9% identity, or more) to the nucleic acid sequence of wild-type human CFB, which is set forth in NCBI Reference No. NM_001710.5 (SEQ ID NO: 12).

As used herein, the term “complement pathway activation or dysregulation” refers to an aberration in the ability of the complement pathway, including the classical pathway, alternative pathway, and lectin pathway, to provide host defense against pathogens and clear immune complexes and damaged cells and for immunoregulation. Alternative complement pathway activation or dysregulation can occur in the fluid phase and at the cell surface and can lead to excessive complement activation or insufficient regulation, both causing tissue injury.

As used herein, “complementary,” when used to describe a first nucleotide or nucleoside sequence in relation to a second nucleotide or nucleoside sequence, refers to the ability of an oligonucleotide comprising the first nucleotide or nucleoside sequence to hybridize and form a duplex structure under certain conditions with an oligonucleotide comprising the second nucleotide sequence, as will be understood by a skilled person in the art. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides. “Complementary” sequences, as used herein, can also include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G: U Wobble or Hoogstein base pairing. Complementary sequences within an oligonucleotide (e.g., RNAi oligonucleotide), or between an oligonucleotide and a target sequence, as described herein, include base-pairing of the oligonucleotide comprising a first nucleotide or nucleoside sequence to an oligonucleotide comprising a second nucleotide or nucleoside sequence over the entire length of one or both nucleotide or nucleoside sequences. Such sequences can be referred to as “fully complementary” with respect to each other herein. Where a first sequence is referred to as “substantially complementary” with respect to a second sequence, the two sequences can be fully complementary or they can form one or more, but generally not more than 5, 4, 3, or 2, mismatched base pairs upon hybridization for a duplex of up to 30 base pairs, while retaining the ability to hybridize under the conditions most relevant to their ultimate application, e.g., reduction of expression via a RISC pathway. “Substantially complementary” can also refer to an oligonucleotide that is substantially complementary to a contiguous portion of the mRNA of interest (e.g., an mRNA encoding a CFB). For example, an oligonucleotide is complementary to at least a part of a CFB mRNA if the sequence is substantially complementary to a non-interrupted portion of an mRNA encoding CFB. However, where two oligonucleotides are designed to form, upon hybridization, one or more single stranded overhangs, such overhangs shall not be regarded as mismatches with regard to the determination of complementarity. For example, an oligonucleotide (e.g., RNAi oligonucleotide) comprising one oligonucleotide of 22 linked nucleosides in length and another oligonucleotide of 20 nucleosides in length, can be referred to as “fully complementary” for the purposes described herein even though they have different lengths.

As used herein, “complementary oligonucleotides” are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two oligonucleotides may hybridize to each other even if they are not completely complementary to each other, provided that each has at least one region that is substantially complementary to the other.

The phrase “contacting a cell with an oligonucleotide,” as used herein, includes contacting a cell with an oligonucleotide, such as an RNAi oligonucleotide (e.g., a single-stranded oligonucleotide or a double-stranded oligonucleotide that forms a duplex), by methods known in the art. Contacting a cell with an oligonucleotide includes contacting a cell in vitro with the oligonucleotide or contacting a cell in vivo with the oligonucleotide. The contacting may be done directly or indirectly. Thus, for example, the oligonucleotide may be put into physical contact with the cell by the individual performing the method, or alternatively, the oligonucleotide agent may be put into a situation that will permit or cause it to subsequently come into contact with the cell. Contacting a cell in vitro may be done, for example, by incubating the cell with the oligonucleotide. Contacting a cell in vivo may be done, for example, by injecting the oligonucleotide into or near the tissue where the cell is located, or by injecting the oligonucleotide agent into another area, e.g., the bloodstream or the subcutaneous space, such that the agent will subsequently reach the tissue where the cell to be contacted is located. For example, the oligonucleotide may contain and/or be coupled to a ligand that directs the oligonucleotide to a site of interest or may be integrated into a vector (e.g., a viral vector) that delivers the oligonucleotide to the target site of interest. Combinations of in vitro and in vivo methods of contacting are also possible. For example, a cell may also be contacted in vitro with an oligonucleotide and subsequently transplanted into a subject.

The term “contiguous nucleobase region” refers to a region of an oligonucleotide (e.g., the antisense strand of an RNAi oligonucleotide) that is complementary to a target nucleic acid. The term may be used interchangeably herein with the term “contiguous nucleotide sequence” or “contiguous nucleobase sequence.” In some embodiments, all of the nucleotides of the oligonucleotide are present in the contiguous nucleotide or nucleoside region. In some embodiments, the oligonucleotide includes the contiguous nucleotide region and may optionally include further nucleotide(s) or nucleoside(s). The nucleotide linker region may or may not be complementary to the target nucleic acid. The internucleoside linkages present between the nucleotides of the contiguous nucleotide region may include phosphorothioate internucleoside linkages. Additionally, the contiguous nucleotide region may include one or more sugar-modified nucleosides.

As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, the term “disease” refers to an interruption, cessation, or disorder of body functions, systems, or organs. Diseases or disorders of interest include those that would benefit from treatment with an oligonucleotide as described herein (e.g., a single-stranded or a double-stranded RNA construct which forms a duplex as described herein) that is targeted to CFB, such as by a treatment method described herein. Non-limiting examples of diseases or disorders mediated by or associated with complement pathway activation or the dysregulation (e.g., dysregulation or activation of CFB) that can be treated using the compositions and methods described herein include, for example, cutaneous disorders, neurological disorders, nephrology disorders, acute care, rheumatic disorders, pulmonary disorders, dermatological disorders, hematologic disorders, and ophthalmic disorders, such as e.g., paroxysmal nocturnal hemoglobinuria (PNH), C3 glomerulopathy (C3G), immunoglobulin A nephropathy (IgAN), membranous nephropathy (MN), including primary MN, E. coli-induced or typical hemolytic uremic syndrome (HUS), atypical hemolytic uremic syndrome (aHUS), age-related macular degeneration, geographic atrophy, diabetic retinopathy, uveitis, intermediate uveitis, Behcet's uveitis, retinitis pigmentosa, macular edema, multifocal choroiditis, Vogt-Koyanagi-Harada syndrome, birdshot retinochoriodopathy, sympathetic ophthalmia, ocular cicatricial pemphigoid, ocular pemphigus, nonarthritic ischemic optic neuropathy, post-operative inflammation, retinal vein occlusion, neurological disorders, multiple sclerosis, stroke, Guillain Barre Syndrome, traumatic brain injury, Parkinson's disease, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, arthritis, autoimmune heart disease, inflammatory bowel disease, ischemia-reperfusion injuries, Barraquer-Simons Syndrome, hemodialysis, anti-neutrophil cytoplasmic antibody (ANCA) vasculitis, cryoglobulinemia, psoriasis, transplantation, diseases of the central nervous system such as Alzheimer's disease and other neurodegenerative conditions, dense deposit disease, blistering cutaneous diseases, membranoproliferative glomerulonephritis type II (MPGN II), chronic graft vs. host disease, Felty syndrome, pyoderma gangrenosum (PG), hidradenitis suppurativa (HS), pulmonary arterial hypertension, primary Sjogren's syndrome, primary biliary cholangitis, autosomal dominant polycystic kidney disease, and myelin oligodendrocyte glycoprotein antibody disease (MOGAD).

As used herein, the term “duplex,” in reference to nucleic acids (e.g., oligonucleotides), refers to a structure formed through complementary base pairing of two antiparallel sequences of nucleotides.

As used herein, the terms “effective amount,” “therapeutically effective amount,” and “a “sufficient amount” of an agent (e.g., an oligonucleotide described herein) that reduces the level and/or activity of CFB (e.g., in a cell or a subject) refers to a quantity sufficient to, when administered to the subject, including a human, effect beneficial or desired results, including clinical results, and, as such, an “effective amount” or synonym thereto depends on the context in which it is being applied. For example, in the context of treating a disease associated with complement pathway activation or dysregulation, it is an amount of the agent that reduces the level and/or activity of CFB sufficient to achieve a treatment response as compared to the response obtained without administration of the agent that reduces the level and/or activity of CFB. The amount of a given agent that reduces the level and/or activity of CFB described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of CFB of the present disclosure is an amount which results in a beneficial or desired result in a subject as compared to a control. As defined herein, a therapeutically effective amount of an agent that reduces the level and/or activity of CFB of the present disclosure may be readily determined by one of ordinary skill by routine methods known in the art. Dosage regimen may be adjusted to provide the optimum therapeutic response.

As used herein, the term “excipient” refers to a non-therapeutic agent that may be included in a composition, for example, to provide or contribute to a desired consistency or stabilizing effect.

“G,” “C,” “A,” “T,” and “U” each generally stand for a nucleotide that contains guanine, cytosine, adenine, thymidine, and uracil as a base, respectively, but may include alternative sugar moieties in addition to ribose and deoxyribose. It is also understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of an oligonucleotide featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure. As used herein, the term “inhibitor” refers to any agent which reduces the level and/or activity of a protein (e.g., CFB). Non-limiting examples of inhibitors include oligonucleotides (e.g., dsRNA, SIRNA, or shRNA). The term “reducing,” as used herein, is used interchangeably with “silencing,” “downregulating,” “suppressing,” and other similar terms, and includes any level of reduction by 5% or more (e.g., 10%, 15%, 25%, 35%, 50%, 75%, and 100%). The typical level of CFB protein found in serum in healthy humans is about 200 μg/mL; therefore, a reduced level of CFB protein may be, e.g., an amount that is less than about 200 μg/mL (e.g., 5 μg/mL, 25 μg/mL, 50 μg/mL, 100 μg/mL, 150 μg/mL, and 190 μg/mL)

By “level” is meant a level or activity of a protein, or mRNA encoding the protein (e.g., CFB), optionally as compared to a reference. The reference can be any useful reference, as defined herein. By a “decreased level” or an “increased level” of a protein is meant a decrease or increase in protein level, respectively, as compared to a reference (e.g., a decrease or an increase of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more, e.g., as compared to a reference; a decrease or an increase of more than about 10%, about 15%, about 20%, about 50%, about 75%, about 100%, or about 200%, e.g., as compared to a reference; a decrease or an increase by less than about 0.01-fold, about 0.02-fold, about 0.1-fold, about 0.3-fold, about 0.5-fold, about 0.8-fold, or less, e.g., as compared to a reference; or a decrease or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., as compared to a reference). A level of a protein or mRNA may be expressed in mass/vol (e.g., g/dL, mg/mL, μg/mL, ng/mL) or percentage relative to total protein or mRNA in a sample.

As used herein the term, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer or in a cell), the two antiparallel regions, which flank the unpaired region, hybridize to form a duplex (referred to as a “stem”).

As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. A “nicked tetraloop structure” is a structure of an RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand. The nicked tetraloop structure causes a single break in the nucleotides of the sense and antisense strands, such that they are no longer joined at that site by a covalent linkage.

The terms “nucleobase” and “base” include the purine (e.g., adenine and guanine) and pyrimidine (e.g., uracil, thymine, and cytosine) moieties present in nucleosides and nucleotides which form hydrogen bonds in nucleic acid hybridization. In the context of the present disclosure, the term nucleobase also encompasses alternative nucleobases which may differ from naturally-occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are, for example, described in Hirao et al. (Accounts of Chemical Research, vol. 45: page 2055, 2012) and Bergstrom (Current Protocols in Nucleic Acid Chemistry Suppl. 37 1.4.1, 2009).

The term “nucleoside” refers to a monomeric unit of or an oligonucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

A “nucleotide,” as used herein, refers to a monomeric unit of an oligonucleotide that comprises a nucleoside and an internucleosidic linkage. The internucleosidic linkage may or may not include a phosphate linkage. Similarly, “linked nucleosides” may or may not be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

As used herein, the term “oligonucleotide” refers to a short nucleic acid, e.g., of less than 100 nucleotides in length. An oligonucleotide may be single-stranded or RNAi. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded SIRNA. In some embodiments, an oligonucleotide is an RNAi oligonucleotide.

As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of an oligonucleotide (e.g., RNAi oligonucleotide). In certain embodiments, the overhang is a 3′ or 5′ overhang on the antisense strand or sense strand of an oligonucleotide (e.g., RNAi oligonucleotide).

As used herein, the term “patient in need thereof” or “subject in need thereof,” refers to the identification of a subject based on need for treatment of a disease or disorder, such as a disease mediated by alternative complement dysregulation (e.g., dysregulation related to CFB, such as dysregulation of one or all of the complement pathways (e.g., alternative, classical, and/or lectin pathways)). A subject can be identified, for example, as having a need for treatment of a disease or disorder, e.g., based upon an earlier diagnosis by a person of skill in the art (e.g., a physician).

“Percent (%) sequence identity” with respect to a reference oligonucleotide or polypeptide sequence is defined as the percentage of nucleic acids or amino acids in a candidate sequence that are identical to the nucleic acids or amino acids in the reference oligonucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid. The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “pharmaceutical composition,” as used herein, represents a composition containing a compound (e.g., an oligonucleotide agent) as described herein formulated with a pharmaceutically acceptable excipient, and optionally manufactured or sold with the approval of a governmental regulatory agency as part of a therapeutic regimen for the treatment of disease in a mammal. Pharmaceutical compositions can be formulated, for example, for subcutaneous administration, for intravenous administration (e.g., as a sterile solution free of particulate emboli and in a solvent system suitable for intravenous use); for intrathecal injection; for intracerebroventricular injections; for intraparenchymal injection; for oral administration in unit dosage form (e.g., a tablet, capsule, caplet, gelcap, or syrup); for topical administration (e.g., as a cream, gel, lotion, or ointment; or in any other pharmaceutically acceptable formulation.

As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, US 2019/0177729, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), Nucleic Acids Res., 43 (6): 2993-3011, the contents of each of which relating to phosphate analogs are incorporated herein by reference).

The term “probe,” as used herein, refers to any molecule that is capable of selectively binding to a specific sequence, e.g., a nucleic acid molecule, such as an mRNA. Probes can be synthesized using well-known and conventional methods of the art or derived from appropriate biological preparations. Probes may be specifically designed to be labeled. Examples of molecules that can be utilized as probes include, but are not limited to, RNA, DNA, proteins, antibodies, and organic molecules.

As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with an RNAi oligonucleotide (e.g., one having an antisense strand that is complementary to CFB mRNA sequence) may result in a decrease in the amount of RNA transcript, protein and/or activity (e.g., encoded by the CFB gene) compared to a cell that is not treated with the RNAi oligonucleotide. Similarly, “reducing expression” as used herein refers to an act that results in reduced expression of a gene (e.g., CFB). The reduction in expression can be assessed by a decrease in the serum concentration of CFB, as described herein (e.g., relative to, e.g., a cell not contacted with an oligonucleotide described herein). Alternatively, the reduction in expression can be assessed by a decrease in the level of transcription and/or translation of CFB mRNA (e.g., a reduction of at least 1%, 2%, 3%, 4%, 5%, 10%, 15%, 20%, 25%, 30%, 40%, 50%, 55%, or 60% or more, such as a reduction in the range of 1%-60% or more, relative to, e.g., a cell not contacted with an oligonucleotide described herein). The reduction in expression of CFB may be measured using a WIESLAB® Complement assay, an ELISA assay, a hemolytic assay, or assay known in the art.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified oligonucleotide or protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., a disease or disorder associated with complement pathway activation or dysregulation); a subject that has been treated with a compound described herein. In some embodiments, the reference sample, standard, or level is matched to the subject sample by at least one of the following criteria: age, weight, sex, disease stage, and overall health. A standard curve of levels of a purified oligonucleotide or protein, e.g., any described herein, within the normal reference range can also be used as a reference.

As used herein, the term “region of complementarity” refers to the region on the antisense strand of an oligonucleotide that is substantially complementary to all or a portion of a gene, primary transcript, a sequence (e.g., a target sequence, e.g., a CFB nucleotide sequence), or processed mRNA, so as to interfere with expression of the endogenous gene (e.g., CFB). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide (e.g., RNAi oligonucleotide).

As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA. In some embodiments, the RNAi oligonucleotide includes a loop region, such as a stem-loop, that contains nucleosides as that term is defined herein.

RNAi oligonucleotide includes, for example, dsRNAs, siRNAs, and shRNAs, which mediate the targeted cleavage of an RNA transcript via an RNA-induced silencing complex (RISC) pathway. RNAi oligonucleotide directs the sequence-specific degradation of mRNA through a process known as RNA interference (RNAi). The RNAi oligonucleotide reduces the expression of C3 in a cell, e.g., a cell within a subject, such as a mammalian subject. In general, the majority of the nucleosides of an RNAi oligonucleotide are ribonucleosides, but as described in detail herein, each or both strands can also include one or more non-ribonucleosides, e.g., deoxyribonucleosides and/or alternative nucleosides. An RNAi oligonucleotide is substantially in a duplex form. In some embodiments, the complementary base-pairing of duplex region(s) of an RNAi oligonucleotide is formed between antiparallel sequences of nucleotides of covalently separate nucleic acid strands. In some embodiments, complementary base-pairing of duplex region(s) of an RNAi oligonucleotide is formed between antiparallel sequences of nucleotides of nucleic acid strands that are covalently linked. In some embodiments, complementary base-pairing of duplex region(s) of an RNAi oligonucleotide is formed from a single nucleic acid strand that is folded (e.g., via a hairpin) to provide complementary antiparallel sequences of nucleotides that base pair together. In some embodiments, an RNAi oligonucleotide comprises two covalently separate nucleic acid strands that are fully duplexed with one another. However, in some embodiments, an RNAi oligonucleotide comprises two covalently separate nucleic acid strands that are partially duplexed, e.g., having overhangs at one or both ends. In some embodiments, an RNAi oligonucleotide comprises an antiparallel sequence of nucleotides that are partially complementary, and, thus, may have one or more mismatches, which may include internal mismatches or end mismatches.

The terms “sense strand” and “passenger strand,” as used herein, refer to the strand of a dsRNA that includes a region that is substantially complementary to a region of the antisense strand. The region of the sense strand that is complementary to a region of the antisense strand is at least 85% (e.g., 86%, 87%, 88%, 89%, 90%, 91%, 92%, 93%, 94%, 95%, 96%, 97%, 98%, 99%, and 100%) identical to a portion of the target gene (e.g., the CFB gene). For example, the sense strand may have a region that is at least 85% identical to a portion of SEQ ID NO: 12, such as, e.g., over at least 10 to 36 nucleotides, e.g., over a length of 10 to 31 nucleotides, 10 to 26 nucleotides, 10 to 20 nucleotides, or 10 to 15 nucleotides.

The terms “siRNA” and “short interfering RNA” also known as “small interfering RNA” refer to an RNA agent, optionally an RNAi agent, of about 10-50 nucleotides in length, the strands optionally having overhanging ends comprising, for example 1, 2 or 3 overhanging linked nucleosides, which is capable of directing or mediating RNA interference. Naturally-occurring siRNAs are generated from longer dsRNA molecules (e.g., >25 linked nucleosides in length) by a cell's RNAi machinery (e.g., Dicer or a homolog thereof).

As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals, such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “sugar” or “sugar moiety,” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

As used herein, the term “stem-loop” refers to a region of an oligonucleotide where two regions have a complementary nucleotide sequence when one is read in the 5′ to 3′ direction and the other is read in the 3′ to 5′ direction and nucleotides between the two regions form an unpaired loop. A stem-loop region may also be referred to as a hairpin or a hairpin loop.

As used herein, the term “strand” refers to an oligonucleotide comprising a chain of linked nucleosides. A “strand comprising a nucleobase sequence” refers to an oligonucleotide comprising a chain of linked nucleosides that is described by the sequence referred to using the standard nucleobase nomenclature.

As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

As used herein, the term “target” or “targeting” refers to an oligonucleotide able to specifically bind to a CFB gene or a CFB mRNA encoding a CFB gene product. For example, it refers to an oligonucleotide able to inhibit said gene or said mRNA (e.g., by reducing the level of protein encoded by the gene or mRNA) by the methods known to those of skill in the art (e.g., in the antisense and RNA interference field).

As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide or to a vector (e.g., a viral vector) containing an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide or vector facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, the term “tetraloop” refers to a loop that increases stability of an adjacent duplex formed by hybridization of flanking sequences of nucleotides. The increase in stability is detectable as an increase in melting temperature (Tm) of an adjacent stem duplex that is higher than the Tm of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base pairing, stacking interactions, hydrogen bonding, and contact interactions (Cheong et al., Nature 1990 Aug. 16; 346 (6285): 680-2; Heus and Pardi, Science 1991 Jul. 12; 253 (5016): 191-4). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) Nucl. Acids Res. 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop. (Woese et al., Proc Natl Acad Sci USA. 1990 November; 87 (21): 8467-71; Antao et al., Nucleic Acids Res. 1991 Nov. 11; 19 (21): 5901-5). Examples of DNA tetraloops include the d (GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, for example: Nakano et al. Biochemistry, 41 (48), 14281-14292, 2002. SHINJI et al. Nippon Kagakkai Koen Yokoshu VOL. 78th; NO. 2; pg. 731 (2000), which are incorporated by reference herein for their relevant disclosures. In some embodiments, the tetraloop is contained within a nicked tetraloop structure.

A “therapeutically-effective amount” or “prophylactically effective amount” refers to an amount (either administered in a single or in multiple doses) of an oligonucleotide composition of the disclosure (e.g., an RNAi oligonucleotide such as a dsRNA) that produces a desired local or systemic effect. e.g., the treatment of one or more symptoms of a disease resulting from complement pathway activation or dysregulation). Oligonucleotides (e.g., RNAi oligonucleotides) employed in the methods of the present disclosure may be administered in a sufficient amount to produce a reasonable benefit/risk ratio applicable to such treatment.

As used herein, the term “treat” refers to the act of providing care to a subject in need thereof, e.g., through the administration a therapeutic agent (e.g., an oligonucleotide described herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom, or contributing factor of a condition (e.g., disease, disorder) experienced by a subject. In some embodiments, the nucleic acid or RNAi oligonucleotide agents (e.g., dsRNAs) described herein are used to control the cellular and clinical manifestations of a disorder of the complement pathway (e.g., a disease or disorder caused by activation or dysregulation of CFB).

### DETAILED DESCRIPTION

Described herein are oligonucleotides (e.g., RNAi oligonucleotides), including sense and antisense strand oligonucleotides, and pharmaceutically acceptable salts thereof, that target complement factor B (CFB), which is known to play a role in alternative complement pathway activation. The oligonucleotides can be administered to decrease the level and/or activity of CFB in a cell (e.g., hepatocytes) or in a subject (e.g., a human). For example, the oligonucleotides can be administered in vivo and can be internalized by a cell (e.g., a hepatocyte; such as by binding to the sialoglycoprotein receptor (ASGPR)). Following cellular internalization, the oligonucleotides can be bound by the RNA-induced silencing complex (RISC) and targeted to CFB mRNA, thereby initiating degradation of the CFB mRNA and blocking translation thereof.

Diseases mediated by alternative complement dysregulation are often a result of complement overactivity. Described herein are methods for treating diseases mediated by, or associated with, complement pathway activation or dysregulation by administration of the oligonucleotides described herein, which reduce the level of expression and/or activity of CFB. Examples of disorders mediated by, or associated with, complement pathway activation or dysregulation that can be treated by the oligonucleotides and compositions described herein include, for example, cutaneous disorders, neurological disorders, nephrology disorders, acute care, rheumatic disorders, pulmonary disorders, dermatological disorders, hematologic disorders, and ophthalmic disorders, such as e.g., paroxysmal nocturnal hemoglobinuria (PNH), C3 glomerulopathy (C3G), immunoglobulin A nephropathy (IgAN), membranous nephropathy (MN), including primary MN, E. coli-induced or typical hemolytic uremic syndrome (HUS), atypical hemolytic uremic syndrome (aHUS), age-related macular degeneration, geographic atrophy, diabetic retinopathy, uveitis, intermediate uveitis, Behcet's uveitis, retinitis pigmentosa, macular edema, multifocal choroiditis, Vogt-Koyanagi-Harada syndrome, birdshot retinochoriodopathy, sympathetic ophthalmia, ocular cicatricial pemphigoid, ocular pemphigus, nonarthritic ischemic optic neuropathy, post-operative inflammation, retinal vein occlusion, neurological disorders, multiple sclerosis, stroke, Guillain Barre Syndrome, traumatic brain injury, Parkinson's disease, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, arthritis, autoimmune heart disease, inflammatory bowel disease, ischemia-reperfusion injuries, Barraquer-Simons Syndrome, hemodialysis, anti-neutrophil cytoplasmic antibody (ANCA) vasculitis, cryoglobulinemia, psoriasis, transplantation, diseases of the central nervous system such as Alzheimer's disease and other neurodegenerative conditions, dense deposit disease, blistering cutaneous diseases, membranoproliferative glomerulonephritis type II (MPGN II), chronic graft vs. host disease, Felty syndrome, pyoderma gangrenosum (PG), hidradenitis suppurativa (HS), pulmonary arterial hypertension, primary Sjogren's syndrome, primary biliary cholangitis, autosomal dominant polycystic kidney disease, and myelin oligodendrocyte glycoprotein antibody disease (MOGAD).

The compositions and methods described herein feature an oligonucleotide (e.g., RNAi oligonucleotide), and pharmaceutically acceptable salts thereof (e.g., a sodium salt thereof), that includes a sense strand and antisense strand, which has substantial sequence identity to a region of the CFB gene (e.g., the human CFB gene).

The oligonucleotide (e.g., RNAi oligonucleotide) can be used to regulate complement pathway activity, e.g., by reducing the level and/or activity of CFB in a cell (e.g., a hepatocyte), such as a cell in a subject (e.g., a human) in need thereof). The oligonucleotide agents target CFB of the complement pathway and leaves activation (protection) of the other pathways of the alternative, classical, and lectin pathways intact. Accordingly, the disclosure features compositions and methods for treating diseases or disorders mediated by complement pathway activation or dysregulation (e.g., diseases or disorders mediated by activation or dysregulation of CFB).

### Complement Factor B Target Sequence

Oligonucleotide-based inhibitors of CFB expression are provided herein that can be used to achieve a therapeutic benefit. Through examination of the CFB mRNA, (see, e.g., Example 1) and in vitro and in vivo testing, it has been discovered that sequences of CFB mRNA are useful as targeting sequences because they are amenable to oligonucleotide-based inhibition. For example, a CFB target sequence can include, or may consist of, a sequence as forth in either of SEQ ID NO: 13 or 14, which corresponds to nucleotides 1827-1845 and 489-507, respectively, of the Homo sapiens complement factor B with Reference Sequence NM_001710.6 (SEQ ID NO: 12). These CFB sequences may be the target sequences of Compound A and Compound B, respectively, and variants thereof described herein that have up to 85% sequence identity thereto. Compounds A and B (and their variants described herein) may also effectively target the Rhesus macaque CFB with Reference Sequences XM_015122636.2. Furthermore, a CFB target sequence can include, or may consist of, a sequence as forth in SEQ ID NO: 31, which corresponds to nucleotides 770-789 of the Mus musculus complement factor B with Reference Sequence NM_008198.2 (SEQ ID NO: 32), which is the target of Compound J (e.g., an RNAi oligonucleotide having the sense sequence of SEQ ID NO: 15 and the antisense sequence of SEQ ID NO: 16). Compound J may also target the Rattus norvegicus complement CFB with Reference Sequence NM_212466.3. These regions of CFB mRNA may be targeted using the oligonucleotide such as the dsRNA agents described herein for purposes of inhibiting CFB mRNA expression and subsequent CFB protein expression.

In some embodiments, the antisense strands of the oligonucleotide (e.g., RNAi oligonucleotide) agents described herein can be designed to have regions of complementarity to CFB mRNA (e.g., within a target sequence of CFB mRNA) for purposes of targeting the mRNA in cells and inhibiting its expression. The region of complementarity is generally of a suitable length and base content to promote annealing of the oligonucleotide (e.g., RNAi oligonucleotide), or a strand thereof, to CFB mRNA for purposes of inhibiting its transcription. The region of complementarity can be at least 11, e.g., at least 12, at least 13, at least 14, at least 15, at least 16, at least 17, at least 18, at least 19 or at least 20 nucleotides in length. For example, an oligonucleotide provided herein may have a region of complementarity to CFB mRNA that is in the range of 12 to 30 (e.g., 12 to 30, 12 to 22, 15 to 25, 17 to 21, 18 to 27, 19 to 27, or 15 to 30) nucleotides in length. Accordingly, the oligonucleotide provided herein may have a region of complementarity to CFB that is 12, 13, 14, 15, 16, 17, 18, 19, 20, 21, 22, 23, 24, 25, 26, 27, 28, 29 or 30 nucleotides in length. In some instances, the oligonucleotide provided herein may have a region of complementarity to the CFB mRNA that is 19 nucleotides in length.

In certain instances, an oligonucleotide agent of the present disclosure may include a region of complementarity (e.g., on an antisense strand of an RNAi oligonucleotide) that is at least partially complementary to a sequence as set forth in SEQ ID NO: 12. For example, an oligonucleotide disclosed herein may comprise a region of complementarity (e.g., on an antisense strand of an RNAi oligonucleotide) that is fully complementary to a sequence as set forth in SEQ ID NO: 12. The region of complementarity of an oligonucleotide (e.g., on an antisense strand of an RNAi oligonucleotide) may be complementary to a contiguous sequence of nucleotides of a sequence as set forth in SEQ ID NO: 12 that is in the range of 12 to 20 nucleotides (e.g., 12 to 20, 12 to 18, 12 to 16, 12 to 14, 14 to 20, 14 to 18, 14 to 16, 16 to 20, 16 to 18, or 18 to 20) in length. In some embodiments, the region of complementarity of an oligonucleotide (e.g., on an antisense strand of an RNAi oligonucleotide) may be complementary to a contiguous sequence of nucleotides of a sequence as set forth in SEQ ID NO: 12 that is 19 nucleotides in length. In certain embodiments, the region of complementarity of an oligonucleotide (e.g., an antisense strand of an RNAi oligonucleotide) may be complementary to a contiguous sequence of nucleotide of a sequence as set forth in SEQ ID NO: 12 that is 20 nucleotides in length.

The region of complementarity of an oligonucleotide that is complementary to contiguous nucleotides of a sequence as set forth in SEQ ID NO: 12 may span a portion of the entire length of an antisense strand. For example, the region of complementarity of an oligonucleotide that is complementary to contiguous nucleotides of a sequence as set forth in SEQ ID NO: 12 may span at least 85% (e.g., at least 86%, at least 90%, at least 95%, and at least 99%) of the entire length of the antisense strand. In certain embodiments, the region of complementarity of the oligonucleotide that is complementary to contiguous nucleotides as set forth in SEQ ID NO:12 may span the entire length of the antisense strand.

The region of complementarity to CFB mRNA may have one or more mismatches as compared with a corresponding sequence of CFB mRNA. For example, a region of complementarity on an oligonucleotide (e.g., an oligonucleotide of 20 to 50 nucleotides in length, such as an oligonucleotide of 20-25 nucleotides in length (e.g., 22 nucleotides in length) may have up to 1, up to 2, up to 3, up to 4, or up to 5 mismatches provided that it maintains the ability to form complementary base pairs with CFB mRNA under appropriate hybridization conditions. Alternatively, a region of complementarity of an oligonucleotide may have no more than 1, no more than 2, no more than 3, no more than 4, or no more than 5 mismatches provided that it maintains the ability to form complementary base pairs with CFB mRNA under appropriate hybridization conditions. If there is more than one mismatch in a region of complementarity, the mismatches may be positioned consecutively (e.g., 2, 3, 4, or 5 in a row) or interspersed throughout the region of complementarity, provided that the oligonucleotide maintains the ability to form complementary base pairs with CFB mRNA under appropriate hybridization conditions. For example, the oligonucleotide agent may include a sense oligonucleotide with the sequence of SEQ ID NO: 4 and variants thereof with up to 1, 2, 3, 4, or 5 mismatches relative to the corresponding CFB sequence of SEQ ID NO: 12, or a corresponding antisense sequence of SEQ ID NO: 6 and variants thereof with up to 1, 2, 3, 4, or 5 mismatches relative to the sequence of SEQ ID NO: 4.

### Types of Oligonucleotides

There are a variety of structures of oligonucleotides that are useful for targeting CFB in the methods of the present disclosure, including RNAi, antisense miRNA, shRNA, and others. Any of the structures described herein or elsewhere may be used as a framework to incorporate or target a sequence described herein (e.g., a hotspot sequence of CFB, such as those of SEQ ID NOs: 13 and 14).

The compositions described herein, which are oligonucleotides (e.g., RNAi oligonucleotides), encode inhibitory constructs (e.g., nucleic acid vectors encoding the same) that target a CFB mRNA (e.g., SEQ ID NO: 12). The oligonucleotides for reducing the expression of CFB expression may engage RNA interference (RNAi) pathways upstream or downstream of dicer involvement. For example, oligonucleotides (e.g., RNAi oligonucleotides) have been developed with 19-25 nucleotides in lengths and with at least one of the sense or antisense strands having a 3′ overhang between 1 and 5 nucleotides (see, e.g., U.S. Pat. No. 8,372,968, which is incorporated herein by reference). Longer oligonucleotides have also been developed that are processed by dicer to generate active RNAi products (see, e.g., U.S. Pat. No. 8,883,996, which is incorporated herein by reference). Furthermore, extended oligonucleotides (e.g., RNAi oligonucleotides) have been produced where either one or both of the 5′ or the 3′ ends of either one or both of the antisense and sense strands are extended beyond a duplex targeting region, such that either the sense strand or the antisense strand includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions on one or both of the 5′ and 3′ ends of the molecule, as well as RNAi extensions.

Additionally, or alternatively, the oligonucleotides provided herein may be designed to engage in the RNA interference pathway downstream of the involvement of dicer, meaning after cleavage by dicer. Such oligonucleotides may have an overhang which includes 1, 2, or 3 nucleotides at the 3′ end of the sense strand. Such oligonucleotides, such as siRNAs, may include a 22-nucleotide guide strand that is antisense to a target RNA (e.g., SEQ ID NO: 13 and 14) and a complementary passenger strand, in which both strands anneal to form a 20-bp duplex and 2 nucleotide overhangs at either or both 3′ ends. Longer oligonucleotide designs are also available, including oligonucleotides having a guide strand of 23 nucleotides and a passenger strand of 21 nucleotides, where there is a blunt end on the 3′-end of passenger strand and 5′-end of the guide strand and a two nucleotide 3′-guide strand overhang on the left side of the molecule 5′-end of the passenger strand and 3′-end of the guide strand. In such molecules, there is a 21 base pair duplex region (see U.S. Pat. Nos. 9,012,138, 9,012,621, and 9,193,753, which are incorporated by reference herein for their disclosure regarding longer oligonucleotides).

The oligonucleotides as disclosed herein may include sense and antisense strands that are both in the range of 17 to 26 (e.g., 17 to 26, 20 to 25, or 21-23) nucleotides in length. For example, an oligonucleotide disclosed herein may include a sense and antisense strand that are both in the range of 19-22 nucleotide in length. The sense and antisense strands may also be of equal length. Alternatively, an oligonucleotide may include sense and antisense strands, such that there is a 3′-overhang on either the sense strand or the antisense strand, or both the sense and antisense strand. For example, the 3′ overhang on the sense, antisense, or both sense and antisense strands may be 1 or 2 nucleotides in length. In some embodiments, the oligonucleotide has an antisense strand of 22 nucleotides and a sense strand of 20 nucleotides, where there is a blunt end on the “right” side of the molecule (i.e., at the 3′-end of the passenger strand and the 5′-end of the guide strand) and a two nucleotide 3′-guide strand overhang on the “left” side of the molecule (i.e., at the 5′-end of the passenger strand and the 3′-end of the guide strand). In such molecules, there may be, e.g., a 20 base pair duplex region.

Other oligonucleotide designs for use with the compositions and methods disclosed herein include, e.g., 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al. Methods Mol. Biol. 2010; 629:141-158), blunt siRNAs (e.g., of 19 bps in length; see: e.g., Kraynack and Baker, RNA Vol. 12, p 163-176 (2006)), asymmetrical siRNAs (aiRNA; see, e.g., Sun et al., Nat. Biotechnol. 26, 1379-1382 (2008)), asymmetric shorter-duplex siRNA (see, e.g., Chang et al., Mol Ther. 2009 April; 17 (4): 725-32), fork siRNAs (see, e.g., Hohjoh, FEBS Letters, Vol 557, issues 1-3; January 2004, p 193-198), single-stranded siRNAs (Elsner; Nature Biotechnology 30, 1063 (2012)), dumbbell-shaped circular siRNAs (see, e.g., Abe et al. J Am Chem Soc 129:15108-15109 (2007)), and small internally segmented interfering RNA (siRNA; see, e.g., Bramsen et al., Nucleic Acids Res. 2007 September; 35 (17): 5886-5897). Each of the foregoing references is incorporated by reference in its entirety for the related disclosures therein. Further non-limiting examples of an oligonucleotide structures that may be used in some embodiments to reduce or inhibit the expression of CFB are microRNA (miRNA), short hairpin RNA (shRNA), and short siRNA (see, Hamilton et al., Embo J., 2002, 21 (17): 4671-4679; see also U.S. Pat. Appln Pub. No. 2009/0099115).

### Oligonucleotides

Oligonucleotides (e.g., RNAi oligonucleotides) for targeting CFB expression via the RNAi pathway generally have a sense strand and an antisense strand that form a duplex with one another. The oligonucleotides (e.g., RNAi oligonucleotides) may be single-stranded or double-stranded ribonucleic acids (dsRNA). Furthermore, the sense and antisense strands may not be covalently linked; for example, the oligonucleotide may be nicked between the sense and antisense strand. The oligonucleotides (e.g., RNAi oligonucleotides) may be in the form of a pharmaceutically acceptable salt. For example, the oligonucleotide (e.g., RNAi oligonucleotide) may be in the form of a sodium salt.

The foregoing oligonucleotide (e.g., RNAi oligonucleotide) sequences are represented as RNA sequences that can be synthesized within the cell; however, these sequences may also be represented as corresponding DNA (e.g., cDNA) that can be incorporated into a vector of the disclosure. One skilled in the art would understand that the cDNA sequence is equivalent to the mRNA sequence, except for the substitution of uridines with thymidines, and can be used for the same purpose herein, i.e., the generation of an antisense oligonucleotide for inhibiting the expression of CFB mRNA. In the case of DNA, the polynucleotide containing the antisense nucleic acid is a DNA sequence. The DNA sequence may correspond to the antisense strand of Compound A or Compound B and may have the polynucleotide sequence of SEQ ID NO: 34 or SEQ ID NO: 36, respectively, or may have at least 85% or more sequence identity thereto. The DNA sequence may correspond to the sense strand of Compound A or Compound B and may have the polynucleotide sequence of SEQ ID NO: 33 or SEQ ID NO: 35, respectively, or may have at least 85% or more sequence identity thereto. In the case of RNA vectors, the transgene cassette incorporates the RNA equivalent of the antisense DNA sequences described herein.

In certain embodiments, the sense strand may include an oligonucleotide sequence having at least 85% (e.g., at least 87%, at least 90%, at least 95%, at least 97%, and at least 99%) sequence identity to SEQ ID NO: 4 or SEQ ID NO: 5. For example, the sense strand may include an oligonucleotide sequence of SEQ ID NO: 4, as in the case of Compound B. In other embodiments, the sense strand may include an oligonucleotide sequence having at least 85% (e.g., at least 87%, at least 90%, at least 95%, at least 97%, and at least 99%) sequence identity to SEQ ID NO: 1 or SEQ ID NO: 2. For example, the sense strand may include an oligonucleotide sequence of SEQ ID NO: 1, as in the case of Compound A.

In some embodiments, the antisense strand may include an oligonucleotides sequence having at least 85% (e.g., at least 87%, at least 90%, at least 95%, at least 97%, and at least 99%) sequence identity to SEQ ID NO: 6. In other embodiments, the antisense strand may include an oligonucleotide sequence having at least 85% (e.g., at least 87%, at least 90%, at least 95%, at least 97%, and at least 99%) sequence identity to SEQ ID NO: 3. For example, the antisense strand may include an oligonucleotide sequence of SEQ ID NO: 6, as in the case of Compound B, and/or the antisense strand may include an oligonucleotide sequence of SEQ ID NO: 3, as in the case of Compound A.

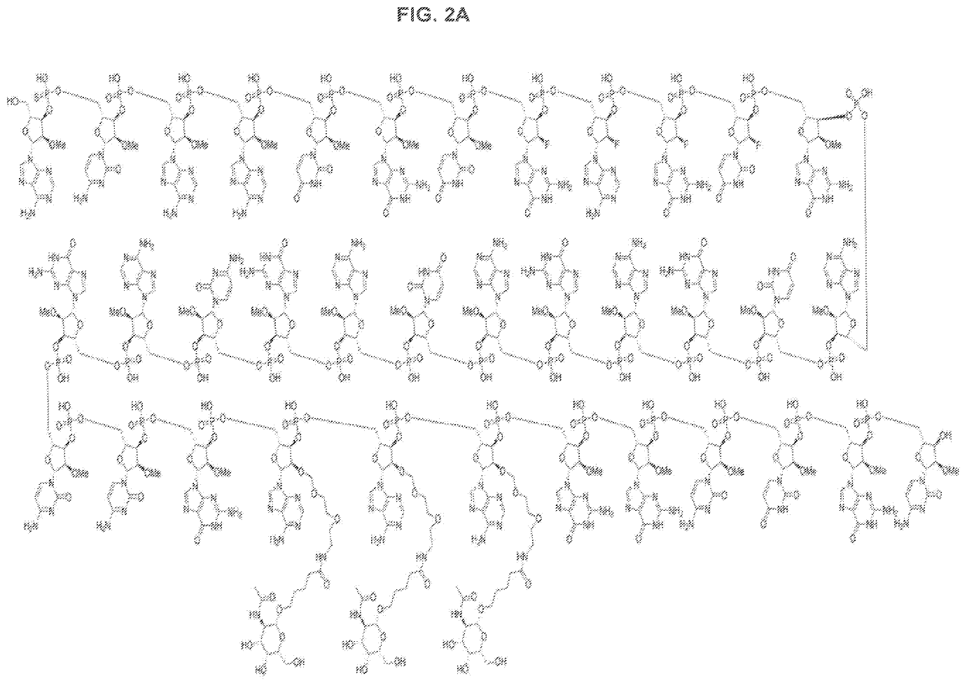

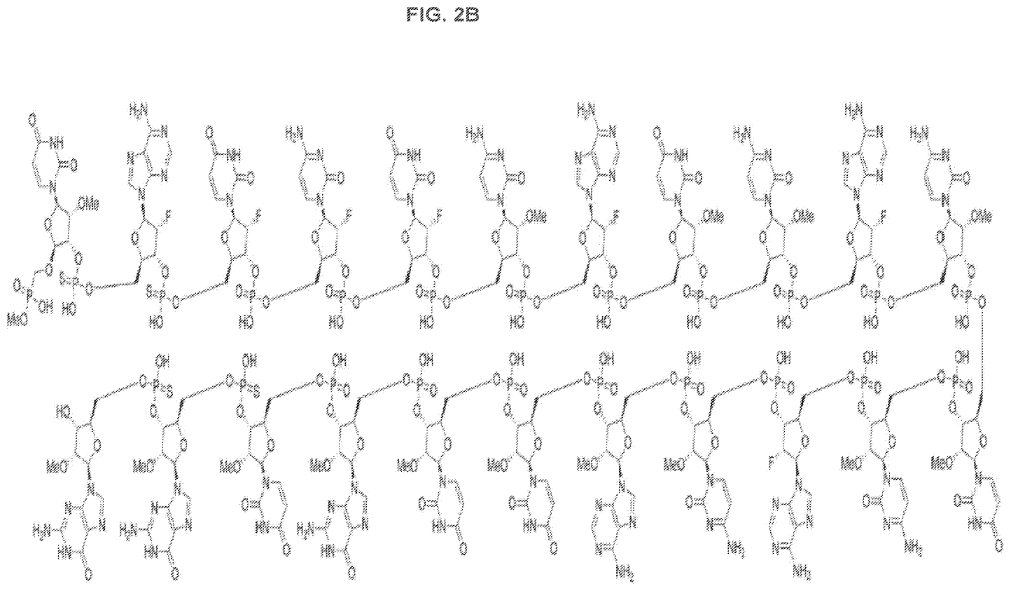

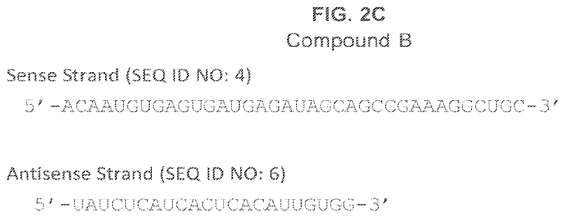

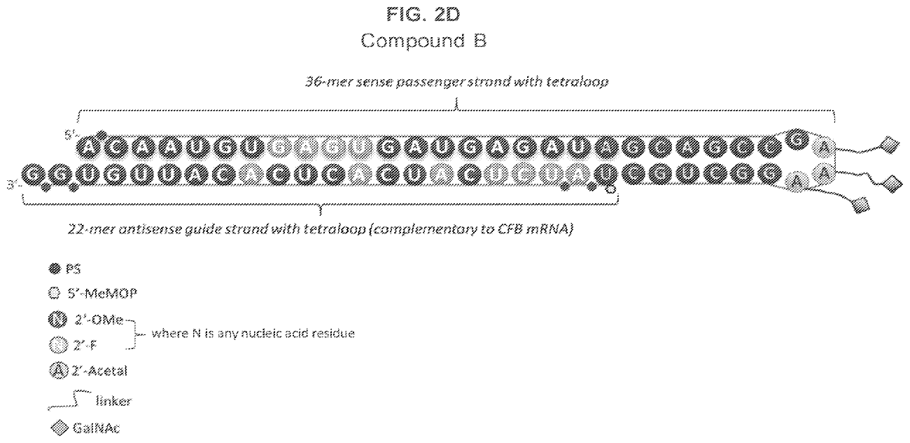

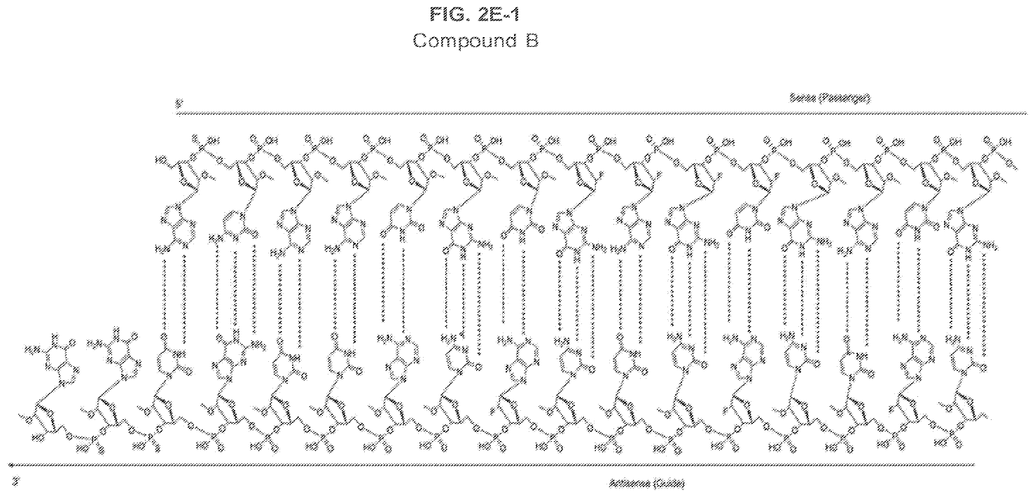

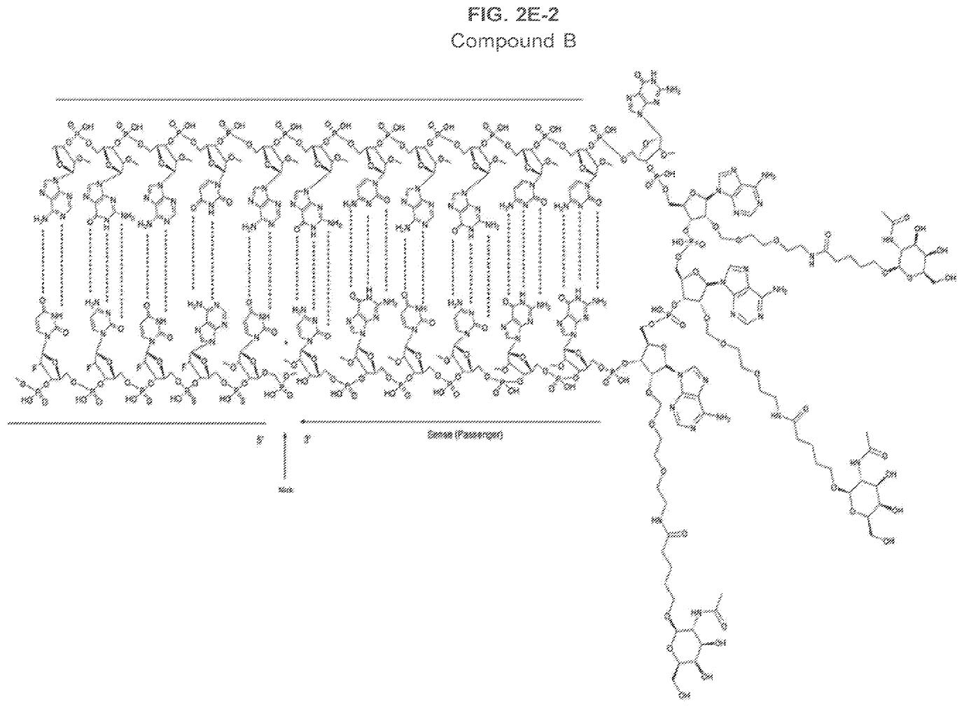

Furthermore, the sense strand may include an oligonucleotide sequence having at least 85% (e.g., at least 87%, at least 90%, at least 95%, at least 97%, and at least 99%) sequence identity to SEQ ID NO: 4 or SEQ ID NO: 5 and the antisense strand may include an oligonucleotide sequence having at least 85% (e.g., at least 87%, at least 90%, at least 95%, at least 97%, and at least 99%) sequence identity to SEQ ID NO: 6. The oligonucleotide (e.g., RNAi oligonucleotide) may contain a sense strand that includes an oligonucleotide sequence of SEQ ID NO: 4 or SEQ ID NO: 5 and an antisense strand that includes an oligonucleotide sequence of SEQ ID NO: 6, as shown for Compound B in FIG. 2C.

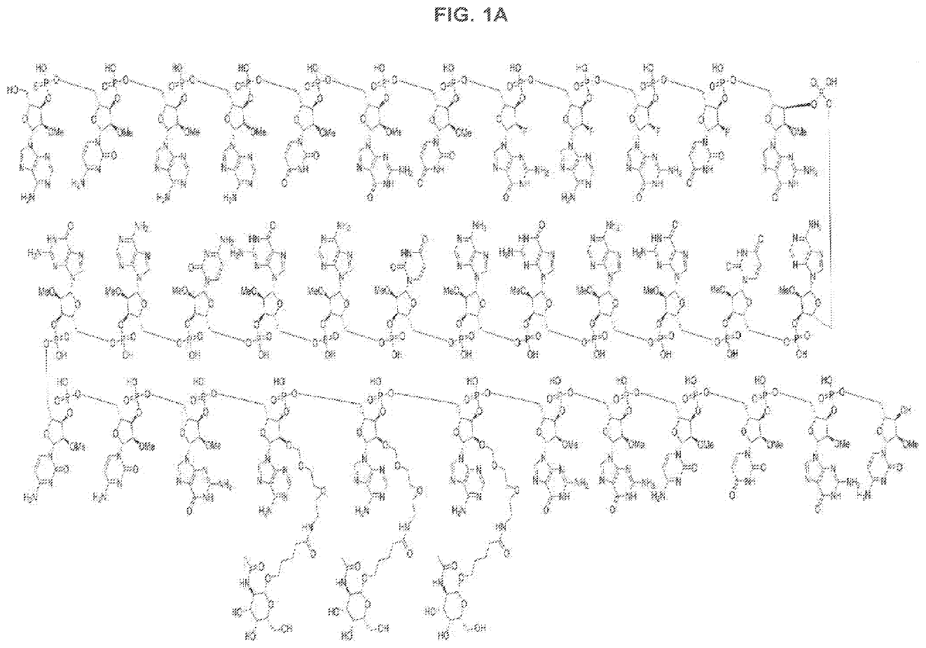

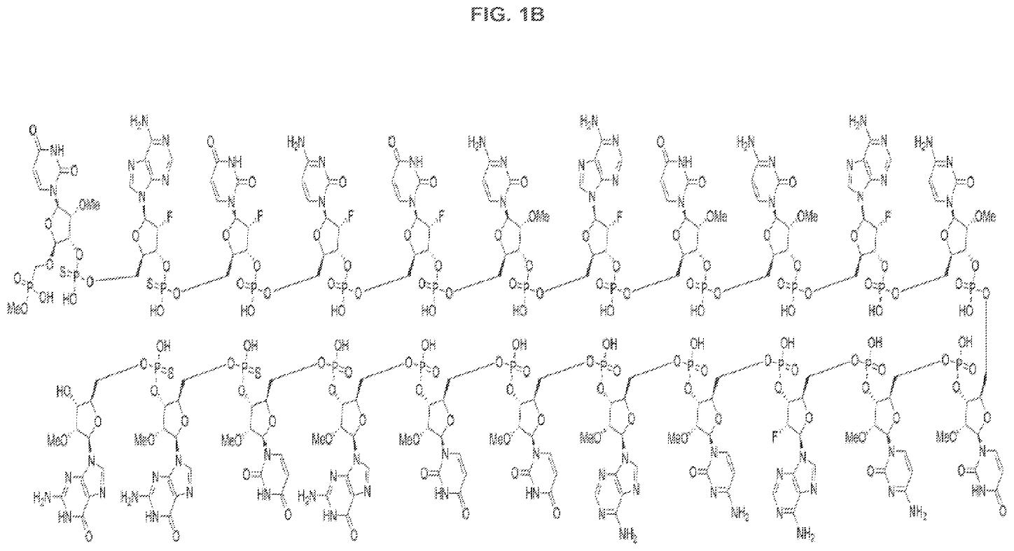

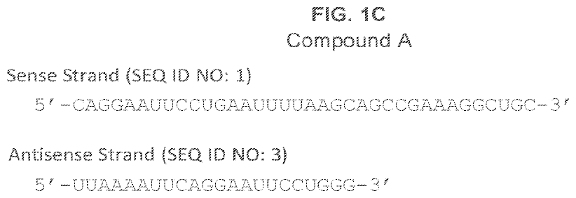

Additionally, the sense strand may include an oligonucleotide sequence having at least 85% (e.g., at least 87%, at least 90%, at least 95%, at least 97%, and at least 99%) sequence identity to SEQ ID NO: 1 or SEQ ID NO: 2 and the antisense strand may include an oligonucleotide sequence having at least 85% (e.g., at least 87%, at least 90%, at least 95%, at least 97%, and at least 99%) sequence identity to SEQ ID NO: 3. Furthermore, the oligonucleotide (e.g., RNAi oligonucleotide) may contain a sense strand that includes an oligonucleotide sequence of SEQ ID NO: 1 or SEQ ID NO: 2 and an antisense strand that includes an oligonucleotide sequence of SEQ ID NO: 3, as shown for Compound A in FIG. 1C.

Furthermore, the sense strand may include an oligonucleotide sequence of SEQ ID NO: 37 and the antisense strand may include an oligonucleotide sequence of SEQ ID NO: 38 as shown below.

Sense Strand (SEQ ID NO: 37):
5′ mA-S-mC-mA-mA-mU-mG-mU-fG-fA-fG-fU-mG-mA-mU-mG-mA-mG-mA-mU-mA-mG-mC-
mA-mG-mC-mC-mG-[ademA-GalNAc]-[ademA-GalNAc]-[ademA-GalNAc]-mG-mG-mC-mU-
mG-mC 3′
hybridized to:
Antisense Strand (SEQ ID NO: 38):
5′ [MePhosphonate-40-mU]-S-fA-S-fU-fC-fU-mC-fA-mU-mC-fA-mC-mU-mC-fA-mC-mA-mU-
mU-mG-mU-S-mG-S-mG 3′

wherein mX is a 2′-O-methyl ribonucleotide, fX is a 2′-fluoro-deoxyribonucleotide, [ademA-GalNAc] is a 2′-O-GalNAc-modified adenosine, [MePhosphonate-40-mU] is a 4′-O-monomethylphosphonate-2′-O-methyl uridine, “-” denotes a phosphodiester linkage, and “-S-” denotes a phosphorothioate linkage as shown in FIGS. 2D, 2E-1 and 2E-2. In some embodiments, the antisense strand may be a pharmaceutically acceptable salt (e.g., a sodium salt) of SEQ ID NO: 38. In some embodiments, the sense strand may be a pharmaceutically acceptable salt (e.g., a sodium salt) of SEQ ID NO: 37.

The sense strand may include an oligonucleotide sequence having at least 85% (e.g., at least 87%, at least 90%, at least 95%, at least 97%, and at least 99%) sequence identity to SEQ ID NO: 15 and the antisense strand may include an oligonucleotide sequence having at least 85% (e.g., at least 87%, at least 90%, at least 95%, at least 97%, and at least 99%) sequence identity to SEQ ID NO: 16. For example, the oligonucleotide (e.g., RNAi oligonucleotide) may contain a sense strand that includes an oligonucleotide sequence of SEQ ID NO: 15 and an antisense strand that includes an oligonucleotide sequence of SEQ ID NO: 16, as shown for Compound J. See Table 1 for examples of sense strand and antisense strand pairs.

TABLE 1
RNAi oligonucleotides targeting CFB mRNA

| | | SEQ | | SEQ | Target sequence |
|---|---|---|---|---|---|
| | | ID | | ID | nucleotide |
| Construct | Sense Strand | NO: | Antisense Strand | NO: | (CFB mRNA) |
| 1 | CAGGAAUUCCUG | 1 | UUAAAAUUCAGGAAUUC | 3 | 1827-1845 of SEQ |
| | AAUUUUAAGCAG | | CUGGG | | ID NO: 12 |
| | CCGAAAGGCUGC | | | | |
| 2 | CAGGAAUUCCUG | 2 | UUAAAAUUCAGGAAUUC | 3 | |
| | AAUUUUAA | | CUGGG | | |
| 3 | ACAAUGUGAGUG | 4 | UAUCUCAUCACUCACAU | 6 | 489-507 of SEQ |
| | AUGAGAUAGCAG | | UGUGG | | ID NO: 12 |
| | CCGAAAGGCUGC | | | | |
| 4 | ACAAUGUGAGUG | 5 | UAUCUCAUCACUCACAU | 6 | |
| | AUGAGAUA | | UGUGG | | |
| 5 | GUGACCAGAUUU | 15 | UUUGAAAAGAAAUCU | 16 | 770-789 of SEQ |
| | CUUUUCAAGCAG | | GGUCACGG | | ID NO: 32 |
| | CCGAAAGGCUGC | | | | |

The oligonucleotide (e.g., RNAi oligonucleotide) includes a duplex region between the sense strand and the antisense strand. The duplex formed between the sense and antisense strand may be between 10 and 30 nucleotides in length (e.g., 10, 11, 12, 13, 14, 15, 16, 17, 18, 19, 20, 21, 22, 23, 24, 25, 26, 27, 28, 29, and 30 nucleotides in length). Accordingly, the duplex formed between a sense and antisense strand may be may between 15 and 25 nucleotides in length (e.g., 15, 16, 17, 18, 19, 20, 21, 22, 23, 24, and 25 nucleotides in length). In some embodiments, the duplex region may be 20 nucleotides in length.

The region on the sense strand that forms a duplex with the antisense strand may have a nucleotide sequence that is at least 85% identical (e.g., 86%, 87%, 88%, 89%, 90%, 91%, 92%, 93%, 94%, 95%, 96%, 97%, 98%, 99%, or more) to the oligonucleotide sequences of either of SEQ ID NOs: 2 and 5. For example, the region on the sense strand that forms a duplex with the antisense strand may have an oligonucleotide sequence of either of SEQ ID NOs: 2 and 5.

Furthermore, a duplex formed between a sense and antisense strand may not span the entire length of the sense strand and/or antisense strand.

The oligonucleotide (e.g., RNAi oligonucleotide) may include a sense strand that is longer than 22 nucleotides (e.g., 23, 24, 25, 26, 27 28, 29, 30, 31, 32, 33, 34, 35, 36, 37, 38, 39 or 40 nucleotides in length), such as a 36-nucleotide sense strand, and an antisense strand that is 18-36 nucleotides in length, such as a 22-nucleotide antisense strand. The oligonucleotide (e.g., RNAi oligonucleotide) has a length such that, when acted upon by a dicer enzyme, the result is an antisense strand that is incorporated into the mature RISC.

The oligonucleotides provided herein may have one 5′ end that is thermodynamically less stable compared to the other 5′ end. The oligonucleotides provided herein may be an asymmetric oligonucleotide that includes a blunt end at the 3′ end of a sense strand and an overhang at the 3′ end of an antisense strand. The 3′ overhang on an antisense strand may be 1-8 nucleotides in length (e.g., 1, 2, 3, 4, 5, 6, 7 or 8 nucleotides in length). For example, the 3′ overhang on the antisense strand may be two nucleotides in length. Typically, an oligonucleotide for RNAi has a two-nucleotide overhang on the 3′ end of the antisense, guide, strand; however, other overhangs are possible. In other embodiments, the 3′ overhang may have a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5, or 6 nucleotides. In some instances, the oligonucleotides may have an overhang on the 5′ end. The overhang may be a 5′ overhang including a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

The two terminal nucleotides on the 3′ end of an antisense strand may be modified. In certain embodiments. The two terminal nucleotides on the 3′ end of the antisense strand may be complementary with the target CFB mRNA. Alternatively, the two terminal nucleotides on the 3′ end of the antisense strand may not be complementary with the target CFB mRNA. In some embodiments, the two terminal nucleotides on the 3′ end of the antisense strand may be GG. Typically, one or both of the two terminal GG nucleotides on each 3′ end of an oligonucleotide is not complementary with the target.

There may be one or more (e.g., 1, 2, 3, 4, 5) mismatches in complementarity between a sense and antisense strand. If there is more than one mismatch between a sense and antisense strand, they may be positioned consecutively (e.g., 2, 3 or more in a row), or interspersed throughout the region of complementarity. For instance, the 3′ end of the sense strand may contain one or more mismatches. Accordingly, two mismatches may be incorporated at the 3′ end of the sense strand. Base mismatches or destabilization of segments at the 3′-end of the sense strand of the oligonucleotide may improve the potency of synthetic duplexes in RNAi, possibly through facilitating processing by dicer.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modifications compared with the specified sequence while retaining essentially same or similar complementary properties as the specified sequence.

### Antisense Strands

The antisense strand of an oligonucleotide may be referred to as a guide strand. For example, if an antisense strand can engage with RNA-induced silencing complex (RISC) and bind to an Argonaute protein, or engage with or bind to one or more similar factors, and direct silencing of a target gene, it may be referred to as a guide strand.

In certain embodiments, the antisense strand is fewer nucleotides in length than the sense strand. In some examples, an oligonucleotide (e.g., RNAi oligonucleotide) provided herein may have an antisense strand including between 10 and 40 nucleotides (e.g., 10, 11, 12, 13, 14, 15, 16, 17,18, 19, 20, 21, 22, 23, 24, 25, 26, 27, 28, 29, 30, 31, 32, 33, 34, 35, 36, 37, 38, 39, and 40 nucleotides) in length. Accordingly, the oligonucleotide (e.g., RNAi oligonucleotide) provided herein may have an antisense strand including between 15 and 30 nucleotides (e.g., 15, 16, 17, 19, 20, 21, 22, 23, 24, 25, 26, 27, 28, 29, and 30 nucleotides) in length. For example, the antisense strand may include between 20 and 25 nucleotides (e.g., 20, 21, 22, 23, 24, and 25 nucleotides) in length. In certain embodiments, the antisense strand may be 22 nucleotides in length.

The oligonucleotide disclosed herein may include an antisense strand including a contiguous sequence between 12 and 22 nucleotides (e.g., 12, 13, 14, 15, 16, 17, 18, 19, 20, 21, and 22 nucleotides) in length that is complementary to a sequence of SEQ ID NO: 12. For example, the oligonucleotide may include an antisense strand including a contiguous sequence of between 15 and 21 nucleotides (e.g., 15, 16, 17, 18, 19, 20, and 21 nucleotides) in length that is complementary to a sequence of SEQ ID NOs: 12. In some embodiments, the oligonucleotide may include an antisense strand having a contiguous sequence of 19 nucleotides in length that is complementary to a sequence of SEQ ID NO: 12.

An oligonucleotide disclosed herein may include an antisense strand having a sequence of either of SEQ ID NOs: 3 or 6. For example, the oligonucleotide disclosed herein may include an antisense strand having the amino acid sequence of SEQ ID NO: 6, as in Compound B as shown in FIG. 2C. In some embodiments, the antisense strand may be a pharmaceutically acceptable salt (e.g., a sodium salt) of SEQ ID NO: 6. SEQ ID NO:6 may have the chemical structure as shown in FIG. 2B. Alternatively, the antisense strand may have a sequence of SEQ ID NO: 3, as in Compound A shown in FIG. 1C. In some embodiments, the antisense strand may be a pharmaceutically acceptable salt (e.g., a sodium salt) of SEQ ID NO: 3.

Additionally, the first position at the 5′ end of antisense strand may be a uridine. The uridine may include a phosphate analog; for example, the uridine may be a 4′-O-monomethylphosphonate-2′-O-methyl uridine.

### Sense Strands

The sense strand of an oligonucleotide may be referred to as a passenger strand. In certain embodiments, the passenger strand is a greater number of nucleotides in length than the guide strand. In some examples, an oligonucleotide (e.g., RNAi oligonucleotide) provided herein may have a sense strand including between 10 and 45 nucleotides (e.g., 10, 11, 12, 13, 14, 15, 16, 17,18, 19, 20, 21, 22, 23, 24, 25, 26, 27, 28, 29, 30, 31, 32, 33, 34, 35, 36, 37, 38, 39, 40, 41, 42, 43, 44, and 45 nucleotides) in length. Accordingly, the oligonucleotide (e.g., RNAi oligonucleotide) provided herein may have a sense strand including between 20 and 50 nucleotides (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 nucleotides) in length. In certain embodiments, the sense strand may be 20 nucleotides in length. In other embodiments, the sense strand may be 36 nucleotides in length.

The oligonucleotide may have a sense strand that includes a contiguous sequence of between 7 to 36 nucleotides in length (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 nucleotides in length) relative to the sequence of SEQ ID NO: 12. Accordingly, the sense strand may include a contiguous sequence between 10 and 30 nucleotides in length (e.g., 10, 11, 12, 13, 14, 15, 16, 17, 18, 19, 20, 21, 22, 23, 24, 25, 26, 27, 28, 29, and 30 nucleotides in length) relative to the sequence of SEQ ID NO: 12. In some embodiments, the oligonucleotides disclosed herein may include a sense strand that includes a contiguous sequence of nucleotides relative to the sequence of SEQ ID NO: 12 that is 19 nucleotides in length.

The sense strand may include a stem-loop at its 3′-end. In some embodiments, a sense strand includes a stem-loop at its 5′ end. The sense strand including a stem-loop may be in the range of 10 to 50 nucleotides in length (e.g., 10, 11, 12, 13, 14, 15, 16, 17, 18, 19, 20, 21, 22, 23, 24, 25, 26, 27,28, 29, 30, 31, 32, 33, 34, 35, 36, 37, 38, 39, 40, 41, 42, 43, 44, 45, 46, 47, 48, 49, and 50 nucleotides in length). Accordingly, the sense strand including a stem-loop may be in the range of 20 to 40 nucleotides in length (e.g., 20, 21, 22, 23, 24, 25, 26, 27,28, 29, 30, 31, 32, 33, 34, 35, 36, 37, 38, 39, and 40 nucleotides in length). For example, the sense strand including a stem-loop may be 36 nucleotides in length.

Furthermore, the stem-loop region on the sense strand may form a duplex region with itself. The duplex region included in stem-loop maybe 1, 2, 3, 4, 5, 6, 7, 8, 9, 10, 11, 12, 13, or 14 nucleotides in length. For example, the duplex region included in the stem-loop may be 6 nucleotides in length. A stem-loop may provide the oligonucleotide agent with protection against degradation (e.g., enzymatic degradation) and may facilitate targeting characteristics for delivery to a target cell. For example, a loop may provide added nucleotides on which modification can be made without substantially affecting the gene expression inhibition activity of an oligonucleotide. In certain embodiments, an oligonucleotide provided herein in which the sense strand includes (e.g., at its 3′-end) a stem-loop set forth as: S1-L-S2, in which S1 is complementary to S2, and in which L forms a loop between S1 and S2 of up to 10 nucleotides in length (e.g., 1, 2, 3, 4, 5, 6, 7, 8, 9, or 10 nucleotides in length). Accordingly, the loop between S1 and S2 may be 4 nucleotides in length, forming a tetraloop, as described herein. In some embodiments, the S1 region is 6 nucleotides in length, the S2 regions is 6 nucleotides in length, and the L region is a 4 nucleotide tetraloop.

The sense strand of the oligonucleotide (e.g., RNAi oligonucleotide) may include a stem-loop region and a region that forms a duplex with the antisense strand. The stem-loop region may include a nucleotide sequence that is at least 85% identical (e.g., 86%, 87%, 88%, 89%, 90%, 91%, 92%, 9%, 94%, 95%, 96%, 97%, 98%, 99%, or more) to the oligonucleotide sequence of SEQ ID NO: 7. In some embodiments, the stem-loop region has the oligonucleotide sequence of SEQ ID NO: 7.

The loop (L) of a stem-loop may a be tetraloop (e.g., within a nicked tetraloop structure). The loop of the stem-loop may have the nucleotide sequence of GAAA. The tetraloop may contain ribonucleotides, deoxyribonucleotides, modified nucleotides, and combinations thereof. Typically, a loop of a stem-loop has 4 to 5 nucleotides. However, in some embodiments, a loop of a stem-loop may include 3 to 6 nucleotides. For example, the loop of the stem-loop may include 3, 4, 5, or 6 nucleotides. The loop of the stem-loop may include a combination of guanosine and adenosine nucleic acid residues.

An oligonucleotide disclosed herein may include a sense strand sequence having a polynucleotide sequence of any one of SEQ ID NOs: 1, 2, 4, and 5. The sense strand may have a sequence of SEQ ID NO: 4, as in Compound B shown in FIG. 2C. SEQ ID NO: 4 may have the chemical structure as shown in FIG. 2A. In some embodiments, the sense strand may be a pharmaceutically acceptable salt (e.g., a sodium salt) of SEQ ID NO: 4. Alternatively, the sense strand may have a nucleotide sequence of SEQ ID NO: 1, as in Compound A shown in FIG. 1C. SEQ ID NO: 1 may have the chemical structure as shown in FIG. 1A. In some embodiments, the sense strand may be a pharmaceutically acceptable salt (e.g., a sodium salt) of SEQ ID NO: 1.

### Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use, see, Bramsen et al., Nucleic Acids Res., 2009, 37, 2867-2881; Bramsen et al., Frontiers in Genetics, 3 (2012): 1-22). Accordingly, in some embodiments, oligonucleotides of the present disclosure may include one or more suitable modifications. The modified nucleotide may have a modification in its base or nucleobase, the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in certain embodiments of any of the oligonucleotides provided herein, all, or substantially all, of the nucleotides of an oligonucleotide are modified. In certain embodiments, more than half of the nucleotides are modified. In other embodiments, less than half of the nucleotides are modified. Typically, with naked delivery, every sugar is modified at the 2′-position. These modifications may be reversible or irreversible. The oligonucleotide as disclosed herein may have a number and type of modified nucleotides sufficient to cause the desired characteristic (e.g., protection from enzymatic degradation, capacity to target a desired cell after in vivo administration, and/or thermodynamic stability).

### Sugar Modifications

A modified sugar, also referred herein to a sugar analog, includes a modified deoxyribose or ribose moiety, in which one or more modifications occur at the 2′, 3′, 4′, and/or 5′ carbon position of the sugar. The modified sugar may also include non-natural alternative carbon structures such as those present in locked nucleic acids (“LNA”) (see, Koshkin et al. (1998), Tetrahedron 54, 3607-3630), unlocked nucleic acids (“UNA”) (see, Snead et al. (2013), Molecular Therapy-Nucleic Acids, 2, e103), and bridged nucleic acids (“BNA”) (see, Imanishi and Obika (2002), The Royal Society of Chemistry, Chem. Commun., 1653-1659). Koshkin et al., Snead et al., and Imanishi and Obika are incorporated by reference herein for their disclosures relating to sugar modifications.

A nucleotide modification at a sugar may include a 2′-modification. A 2′-modification may be 2′-aminoethyl, 2′-fluoro, 2′-O-methyl, 2′-O-methoxyethyl, and 2′-deoxy-2′-fluoro-β-d-arabinonucleic acid. Typically, the modification is 2′-fluoro, 2′-O-methyl, or 2′-O-methoxyethyl. In some embodiments, the modification is a 2′-fluoro and/or a 2′-O-methyl. A modification at a sugar may include a modification of the sugar ring, which may have a modification of one or more carbons of the sugar ring. For example, a modification of a sugar of a nucleotide may include a 2′-oxygen of a sugar linked to a 1′-carbon or 4′-carbon of the sugar, or a 2′-oxygen linked to the 1′-carbon or 4′-carbon via an ethylene or methylene bridge. In certain embodiments, a modified nucleotide may have an acyclic sugar that lacks a 2′-carbon to 3′-carbon bond. In some embodiments, a modified nucleotide may have a thiol group, e.g., in the 4′ position of the sugar.

The oligonucleotide (e.g., RNAi oligonucleotide) described herein may include at least one modified nucleotide (e.g., at least 1, at least 5, at least 10, at least 15, at least 20, at least 25, at least 30, at least 35, at least 40, at least 45, at least 50, at least 55, at least 60, or more). For example, the sense strand of the oligonucleotide may include at least one modified nucleotide (e.g., at least 1, at least 5, at least 10, at least 15, at least 20, at least 25, at least 30, at least 35, or more). Also, for example, the antisense strand of the oligonucleotide may include at least one modified nucleotide (e.g., at least 1, at least 5, at least 10, at least 15, at least 20, or more).

In certain embodiments, the oligonucleotide (e.g., RNAi oligonucleotide) described herein may contain between 20 and 50 (e.g., 20 to 30, 24 to 30, 28 to 30, 30 to 40, 34 to 40, 38 to 44, 44 to 50, and 48 to 50) modified nucleotides.

All of the nucleotides of the sense strand of the oligonucleotide may be modified. Furthermore, all of the nucleotides of the antisense strand of the oligonucleotide may be modified. In some embodiments, all of the nucleotides of the oligonucleotide (e.g., RNAi oligonucleotide) including both the sense strand and the antisense strand are modified. The modified nucleotide may be a 2′-modification (e.g., a 2′-fluoro or 2′-O-methyl). The 2′-modification to the nucleotide may be a 2′-fluoro and/or a 2′-O-methyl, wherein optionally the 2′-fluoro modification is 2′-fluoro deoxyribonucleoside and/or the 2′-O-methyl modification is 2′-O-methyl ribonucleoside.

The disclosure provides oligonucleotides having different modification patterns. The oligonucleotide including the sense strand and the antisense strand may include between 40 and 50 (e.g., 41, 2, 43, 44, 45, 46, 47, 48, and 49) 2′-O-methyl modifications. The modified oligonucleotides may include a sense strand having a nucleotide sequence of either of SEQ ID NO: 1 or 4, and an antisense strand having a nucleotide sequence of either of SEQ ID NO: 3 or 6 (e.g., the oligonucleotide agent may have a sense strand of SEQ ID NO: 4 and an antisense strand of SEQ ID NO: 6, or the oligonucleotide agent may have a sense strand of SEQ ID NO: 1 and an antisense strand of SEQ ID NO: 3). In some embodiments, for these oligonucleotides, one or more of positions 1, 2, 3, 4, 5, 6, 7, 12, 13, 14, 15, 16, 17, 18, 19, 20, 21, 22, 23, 24, 25, 26, 27, 31, 32, 33, 34, 35 and 36 of the sense strand, and/or one or more of positions 1, 4, 6, 8, 9, 11, 12, 13, 15, 16, 17, 18, 19, 20, 21, and 22 of the antisense strand are modified with a 2′-O-methyl modified nucleoside, such as a 2′-O-methyl ribonucleoside. In some embodiments, all of positions 1, 2, 3, 4, 5, 6, 7, 12, 13, 14, 15, 16, 17, 18, 19, 20, 21, 22, 23, 24, 25, 26, 27, 31, 32, 33, 34, 35 and 36 of the sense strand, and all of positions 1, 4, 6, 8, 9, 11, 12, 13, 15, 16, 17, 18, 19, 20, 21, and 22 of the antisense strand are modified with a 2′-O-methyl modified nucleoside, such as a 2′-O-methyl ribonucleoside. In other embodiments, one or more of positions 1, 2, 3, 4, 5, 6, 7, 12, 13, 14, 15, 16, 17, 18, 19, 20, 21, 22, 23, 24, 25, 26, 27, 31, 32, 33, 34, 35 and 36 of the sense strand, and/or one or more of positions 1, 6, 8, 9, 11, 12, 13, 15, 16, 17, 18 19, 20, 21, and 22 of the antisense strand are modified with a 2′-O-methyl modified nucleoside, such as a 2′-O-methyl ribonucleoside. In certain embodiments, all of positions 1, 2, 3, 4, 5, 6, 7, 12, 13, 14, 15, 16, 17, 18, 19, 20, 21, 22, 23, 24, 25, 26, 27, 31, 32, 33, 34, 35 and 36 of the sense strand, and/or all of positions 1, 6, 8, 9, 11, 12, 13, 15, 16, 17, 18 19, 20, 21, and 22 of the antisense strand are modified with a 2′-O-methyl modified nucleoside, such as a 2′-O-methyl ribonucleoside.

The oligonucleotide including the sense strand and the antisense strand may have between 5 and 15 (e.g., 6, 7, 8, 9, 10, 11, 12, 13, and 14) 2′-fluoro modifications. For these oligonucleotides, one or more of positions 8, 9, 10, and 11 of the sense strand, and/or one or more of positions 2, 3, 4, 5, 7, 10, and 14 of the antisense strand may be modified with a 2′-fluoro modified nucleoside. For example, all of positions 8, 9, 10, and 11 of the sense strand, and/or all of positions 2, 3, 5, 7, 10, and 14 of the antisense strand may be modified with a 2′-fluoro modified nucleoside. In another example, all of positions 8, 9, 10, and 11 of the sense strand, and/or all of positions 2, 3, 4, 5, 7, 10, and 14 of the antisense strand may be modified with a 2′-fluoro modified nucleoside.

For oligonucleotides comprising a sense strand having a sequence of SEQ ID NO: 4, and an antisense strand having a sequence of SEQ ID NO: 6, one or more of positions 1-7, −12-27, and 31-36 of the sense strand, and/or one or more of positions 1, 6, 8, 9, 11-13, and 15-22 of the antisense strand may be modified with a 2′-O-methyl modified nucleoside. Furthermore, all of positions 1-7, 12-27, and 31-36 of the sense strand, and all of positions 1, 6, 8, 9, 11-13, and 15-22 of the antisense strand may be modified with a 2′-O-methyl modified nucleoside. For oligonucleotides with a sense strand having a sequence of SEQ ID NO: 4, and an antisense strand having sequence of SEQ ID NO: 6, one or more of positions 8-11 of the sense strand, and one or more of positions 2, 3, 4, 5, 7, 10, and 14 of the antisense strand may be modified with a 2′-fluoro modified nucleoside. Accordingly, all of positions 8-11 of the sense strand, and all of positions 2, 3, 4, 5, 7, 10, and 14 of the antisense strand may be modified with a 2′-fluoro modified nucleoside.

For example, for oligonucleotides with a sense strand having a sequence of SEQ ID NO: 4, and an antisense strand having a sequence of SEQ ID NO: 6, all of positions 1-7, 12-27, and 31-36 of the sense strand, and all of positions 1, 6, 8, 9, 11-13, and 15-22 of the antisense strand may be modified with a 2′-O-methyl modified nucleoside; and all of positions 8-11 of the sense strand, and all of positions 2, 3, 4, 5, 7, 10, and 14 of the antisense strand may be modified with a 2′-fluoro modified nucleoside, shown in FIGS. 2A and 2B, respectively.

For oligonucleotides comprising a sense strand having a sequence of SEQ ID NO: 1, and an antisense strand having a sequence of SEQ ID NO: 3, one or more of positions 1-7, 12-27, and 31-36 of the sense strand, and/or one or more of positions 1, 4, 6, 8, 9, 11-13, and 15-22 of the antisense strand may be modified with a 2′-O-methyl modified nucleoside. In some embodiments, all of positions 1-7, 12-27, and 31-36 of the sense strand, and all of positions 1, 4, 6, 8, 9, 11-13, and 15-22 of the antisense strand may be modified with a 2′-O-methyl modified nucleoside. Additionally, for oligonucleotides that include a sense strand with a sequence of SEQ ID NO: 1 and an antisense strand with a sequence of SEQ ID NO: 3, one or more of positions 8-11 of the sense strand and/or one or more of positions 2, 3, 5, 7, 10, and 14 of the antisense strand may be modified with a 2′-fluoro modified nucleoside. In some embodiments, all of positions 8-11 of the sense strand, and all of positions 2, 3, 5, 7, 10, and 14 of the antisense strand are modified with a 2′-fluoro modified nucleoside. For example, for oligonucleotides having a sense strand including a sequence of SEQ ID NO: 1 and an antisense strand having a sequence of SEQ ID NO: 3, all of positions 1-7, 12-27, and 31-36 of the sense strand, and all of positions 1, 4, 6, 8, 9, 11-13, and 15-22 of the antisense strand may be modified with a 2′-O-methyl modified nucleoside; and all of positions 8-11 of the sense strand, and all of positions 2, 3, 5, 7, 10, and 14 of the antisense strand may be modified with a 2′-fluoro modified nucleoside, as shown in FIGS. 1A and 1B, respectively.

In some embodiments, the terminal 3′-end group (e.g., a 3′-hydroxyl) may be modified with a phosphate group or other group, which can be used, for example, to attach linkers, adapters, or labels or for the direct ligation of an oligonucleotide to another nucleic acid.

### 5′ Terminal Phosphates

The 5′-terminal phosphate groups of the oligonucleotide (e.g., RNAi oligonucleotide) may enhance the interaction with Argonaute 2. In certain embodiments, the oligonucleotide (e.g., RNAi oligonucleotide) includes a uridine at the first position of the 5′ end of the antisense strand. However, oligonucleotides having a 5′-phosphate group may be susceptible to degradation via phosphatases or other enzymes, which can limit their bioavailability in vivo. In some embodiments, oligonucleotides include analogs of 5′ phosphates that are resistant to such degradation. Therefore, the uridine at the 5′ end of the antisense strand may include a phosphate analog. The phosphate analog may be oxymethylphosphonate, vinylphosphonate, or malonylphosphonate. Furthermore, the 5′ end of an oligonucleotide strand may be attached to a chemical moiety that mimics the electrostatic and steric properties of a natural 5′-phosphate group (“phosphate mimic”) (see, Prakash et al., Nucleic Acids Res. 2015 Mar. 31; 43 (6): 2993-3011, the contents of which relating to phosphate analogs are incorporated herein by reference). Many phosphate mimics have been developed that can be attached to the 5′ end (see, U.S. Pat. No. 8,927,513, the contents of which relating to phosphate analogs are incorporated herein by reference). Other modifications have been developed for the 5′ end of oligonucleotides (see, WO 2011/133871, the contents of which relating to phosphate analogs are incorporated herein by reference). In certain embodiments, a hydroxyl group may be attached to the 5′ end of the oligonucleotide.

The oligonucleotide may have a phosphate analog at a 4′-carbon position of the sugar, referred to as a “4′-phosphate analog”. See, for example, WO 2018/045317, the contents of which relating to phosphate analogs are incorporated herein by reference. The oligonucleotide provided herein may include a 4′-phosphate analog at a 5′-terminal nucleotide. In some embodiments, the phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃. In some embodiments, R is CH₃. In some embodiments, the 4′-phosphate analog is 5′-methoxyphosphanate-4′-oxy. In some embodiments, the 4′-phosphate analog is 4′-(methyl methoxyphosphonate). In some embodiments, the phosphate analog is a 4′-O-monomethylphosphonate analog.

In some embodiments, a phosphate analog attached to the oligonucleotide is a methoxy phosphonate (MOP). The phosphate analog attached to the oligonucleotide may be a 5′ monomethyl protected MOP. In some embodiments, the following uridine nucleotide comprising a phosphate analog may be used, e.g., at the first position of the antisense strand:

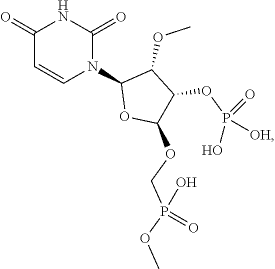 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as 4′-O-monomethylphosphonate-2′-O-methyl uridine ([MePhosphonate-40-mU]) or 5′-Methoxy, Phosphonate-4′oxy-2′-O-methyluridine (5′-MeMOP). The 5′-Methoxy, Phosphonate-4′oxy-2′-O-methyluridine (5′-MeMOP) may be the first nucleotide at the 5′ end of the antisense strand. In some embodiments, the first nucleotide at the 5′ end of either of SEQ ID NOs: 3 or 6 may be a 5′-Methoxy, Phosphonate-4′oxy-2′-O-methyluridine.

### Modified Internucleoside Linkages

Phosphate modifications or substitutions in the oligonucleotide may result in an oligonucleotide that includes at least one (e.g., at least 1, at least 2, at least 3, at least 5, or at least 6) modified internucleotide linkage. Any one of the oligonucleotides disclosed herein may include between 1 and 10 (e.g., 1 to 10, 2 to 8, 4 to 6, 3 to 10, 5 to 10, 1 to 5, 1 to 3 or 1 to 2) modified internucleotide linkages. For example, any one of the oligonucleotides disclosed herein may include 1, 2, 3, 4, 5, 6, 7, 8, 9, or 10 modified internucleotide linkages. In some embodiments, the oligonucleotide (e.g., RNAi oligonucleotide) may include 5 modified internucleotide linkages. For example, the sense strand of the oligonucleotide may include 1 modified internucleotide linkage, and the antisense strand may include 4 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. At least one modified internucleotide linkage of any one of the oligonucleotides as disclosed herein may be a phosphorothioate linkage. In certain embodiments, all of the modified internucleotide linkages of the oligonucleotide may be phosphorothioate linkages.

The oligonucleotide described herein may have a phosphorothioate linkage between one or more of: positions 1 and 2 of the sense strand, positions 1 and 2 of the antisense strand, positions 2 and 3 of the antisense strand, positions 20 and 21 of the antisense strand, and positions 21 and 22 of the antisense strand. For example, the sense strand of the oligonucleotide may have a phosphorothioate linkage between positions 1 and 2 of the sense strand, positions 1 and 2 of the antisense strand, positions 2 and 3 of the antisense strand, positions 20 and 21 of the antisense strand, and positions 21 and 22 of the antisense strand. Accordingly, the sense strand having a sequence of SEQ ID NO: 1 or 4 may have a phosphorothioate linkage between positions 1 and 2, and the antisense strand having a sequence of SEQ ID NO: 3 or 6 may have a phosphorothioate linkage between positions 1 and 2, 2 and 3, 20 and 21, and 21 and 22.

### Base Modifications

The oligonucleotides provided herein may have one or more modified nucleobases. Modified nucleobases, also referred to herein as base analogs, may be linked at the 1′ position of a nucleotide sugar moiety. The modified nucleobase may be a nitrogenous base. In certain embodiments, the modified nucleobase may contain a nitrogen atom. See, U.S. Published patent application No. 20080274462 the contents of which relating to modified nucleobases are incorporated herein by reference. The modified nucleotide may also include a universal base. However, in certain embodiments, a modified nucleotide may not contain a nucleobase (e.g., abasic).

In some embodiments a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering the structure of the duplex. In some embodiments, compared to a reference single-stranded nucleic acid (e.g., oligonucleotide or polynucleotide) that is fully complementary to a target nucleic acid, a single-stranded nucleic acid containing a universal base forms a duplex with the target nucleic acid that has a lower Tm than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tm than a duplex formed with the nucleic acid comprising the mismatched base. Non-limiting examples of universal-binding nucleotides include inosine, 1-β-D-ribofuranosyl-5-nitroindole, and/or 1-β-D-ribofuranosyl-3-nitropyrrole (see, US 2007/0254362; Van Aerschot et al., Nucleic Acids Res. 1995 Nov. 11; 23 (21): 4363-70; Loakes et al., Nucleic Acids Res. 1995 Jul. 11; 23 (13): 2361-6; Loakes et al., Nucleic Acids Res. 1994 Oct. 11; 22 (20): 4039-43. Each of the foregoing is incorporated by reference herein for their disclosures relating to base modifications).

### Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

A reversibly modified nucleotide may include a glutathione-sensitive moiety. Typically, nucleic acid molecules may be chemically modified with cyclic disulfide moieties to mask the negative charge created by the internucleotide diphosphate linkages and improve cellular uptake and nuclease resistance. See US 2011/0294869 originally assigned to Traversa Therapeutics, Inc. (“Traversa”), PCT Publication No. WO 2015/188197 to Solstice Biologics, Ltd. (“Solstice”), Meade et al., Nature Biotechnology, 2014,32:1256-1263 (“Meade”), PCT Publication No. WO 2014/088920 to Merck Sharp & Dohme Corp, each of which are incorporated by reference for their disclosures of such modifications. The reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (see, Dellinger et al. J. Am. Chem. Soc. 2003, 125:940-950).

Such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. The structure of the glutathione-sensitive moiety may be engineered to modify the kinetics of its release.

In some embodiments, a glutathione-sensitive moiety is attached to the sugar of the nucleotide. In some embodiments, a glutathione-sensitive moiety is attached to the 2′carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., US 2019/0177355, the contents of which are incorporated by reference herein for its relevant disclosures.

### Targeting Ligands

It may be desirable to target the oligonucleotides of the disclosure to one or more cells or one or more organs (e.g., cells of the liver). Such a strategy may help to avoid undesirable effects in other organs or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell, or organ, e.g., to facilitate delivery of the oligonucleotide to the liver. In certain embodiments, oligonucleotides disclosed herein may be modified to facilitate delivery of the oligonucleotide to the hepatocytes of the liver. An oligonucleotide may include a nucleotide that is conjugated to one or more targeting ligand.

A targeting ligand may include a carbohydrate, amino sugar, cholesterol, peptide, polypeptide, protein, or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide (SEQ ID NO: 78) to target tumor vasculature or stoma, transferring, lactoferrin, or an aptamer to target transferrin receptors expressed on CNS vasculature, or an anti-EGFR antibody to target EGFR on glioma cells. In some embodiments, the targeting ligand is one or more N-Acetylgalactosamine (GalNAc) moieties.

One or more (e.g., 1, 2, 3, 4, 5 or 6) nucleotides of an oligonucleotide may be each conjugated to a separate targeting ligand. In some instances, 2 to 4 nucleotides of an oligonucleotide are each conjugated to a separate targeting ligand. The targeting ligands may be conjugated to 2 to 4 nucleotides at either ends of the sense or antisense strand (e.g., the ligand is conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may include a stem-loop at either the 5′ or 3′ end of the sense strand and 1, 2, 3, or 4 nucleotides of the loop of the stem may be individually conjugated to a targeting ligand. In some embodiments, the oligonucleotide includes a stem-loop at the 3′ end of the sense strand and 3 nucleotides of the loop of the stem are individually conjugated to a targeting ligand.

In some embodiments, it is desirable to target an oligonucleotide that reduces the expression of CFB to the hepatocytes of the liver of the subject. Any suitable hepatocyte targeting moiety may be used for this purpose.

GalNAc is a high affinity ligand for asialoglycoprotein receptors (ASGPR), which are primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation, either indirect or direct, of GalNAc moieties to oligonucleotides of the instant disclosure may be used to target these oligonucleotides to the ASGPR expressed on these hepatocyte cells.

For example, an oligonucleotide of the disclosure may be conjugated directly or indirectly to a monovalent GalNAc. The oligonucleotide may be conjugated directly or indirectly to more than one (e.g., 2, 3, 4, or more) monovalent GalNAc, and is typically conjugated to 3 or 4 monovalent GalNAc moieties. The GalNAc moiety(ies) may be present within a loop region of the oligonucleotides described herein. The GalNAc moiety may be used to target the oligonucleotides of the disclosure to ASGPR on hepatocytes; at which point, the GalNAc conjugated oligonucleotide may be internalized and integrated into the intracellular RNAi machinery called the RNA-induced silencing complex (RISC). The RISC Argonaute-2 (Argo-2) protein within this complex targets the antisense strand of the oligonucleotide duplex to its complementary CFB mRNA and initiates its degradation, thus blocking translation of the target.

In some embodiments, 2 to 4 nucleotides of the loop (L) of the stem-loop are each conjugated to a separate GalNAc moiety. In some embodiments, three nucleotides of the loop of the stem-of the oligonucleotide may be conjugated directly or indirectly to three separate monovalent GalNAc moieties. In some embodiments, the oligonucleotide is conjugated to one or more bivalent GalNAc, trivalent GalNAc, or tetravalent GalNAc moieties.

The oligonucleotide described herein may include a monovalent GalNAc attached to a guanine nucleobase, referred to as [ademG-GalNAc] or 2′-aminodiethoxymethanol-guanine-GalNAc, as depicted below:

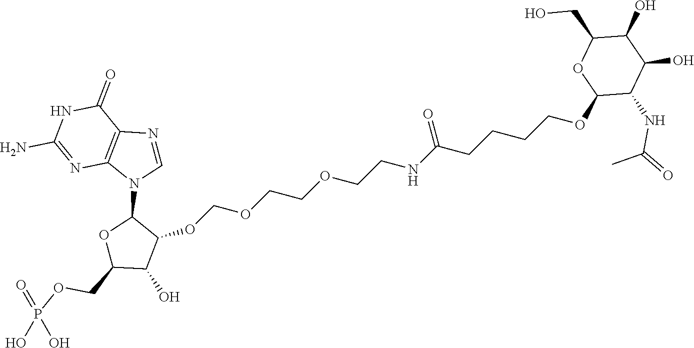 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

Additionally, or alternatively, the oligonucleotide herein may include a monovalent GalNAc attached to an adenine nucleobase, referred to as 2′-O-GalNAc-modified adenosine, [ademA-GalNAc], or 2′-aminodiethoxymethanol-adenine-GalNAc, as depicted below.

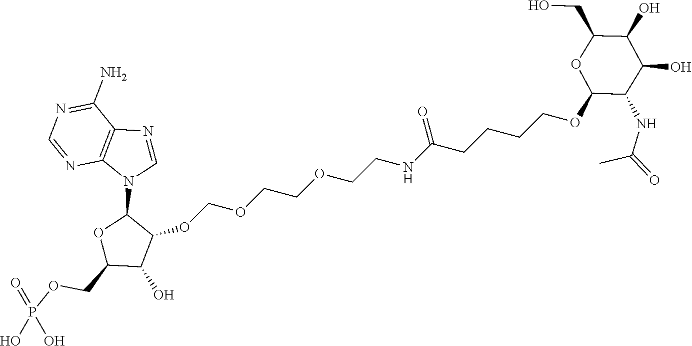

An example of such conjugation is shown below for a loop comprising from 5′ to 3′ the nucleotide sequence GAAA (L=linker, X=heteroatom) stem attachment points are shown. Such a loop may be present, for example, at nucleotide positions 27-30 of the molecule shown in FIGS. 1A and 2A (see also FIGS. 1C and 2C). In the chemical formula

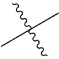

is an attachment point to the oligonucleotide strand.

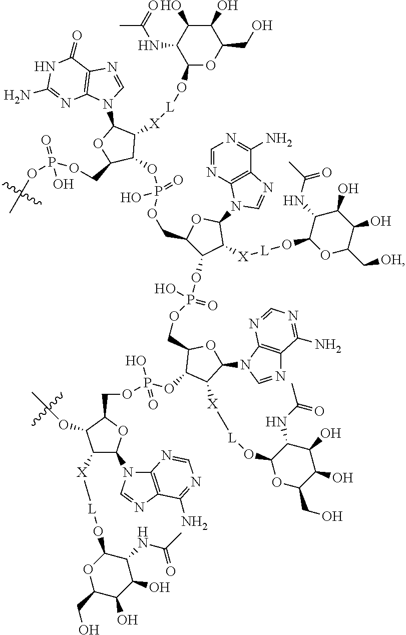

Appropriate methods or chemistry (e.g., click chemistry) can be used to link a targeting ligand to a nucleotide. A targeting ligand may be conjugated to a nucleotide using a click linker. Furthermore, an acetal-based linker may be used to conjugate a targeting ligand to a nucleotide of any one of the oligonucleotides described herein. Acetal-based linkers are disclosed, for example, in International Patent Application Publication Number WO 2016/100401 A1, which published on Jun. 23, 2016, and the contents of which relating to such linkers are incorporated herein by reference. The linker may be a labile linker. However, in other embodiments, the linker is stable (non-labile).

An example is shown below for a loop comprising from 5′ to 3′ the nucleotides GAAA, in which GalNAc moieties are attached to nucleotides of the loop using an acetal linker. Such a loop may be present in an oligonucleotide disclosed herein (see, for example, positions 27-30 of the oligonucleotides having the sequences of SEQ ID NO: 1 and 4). In the chemical formula,

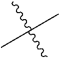

is an attachment point to the oligonucleotide strand.

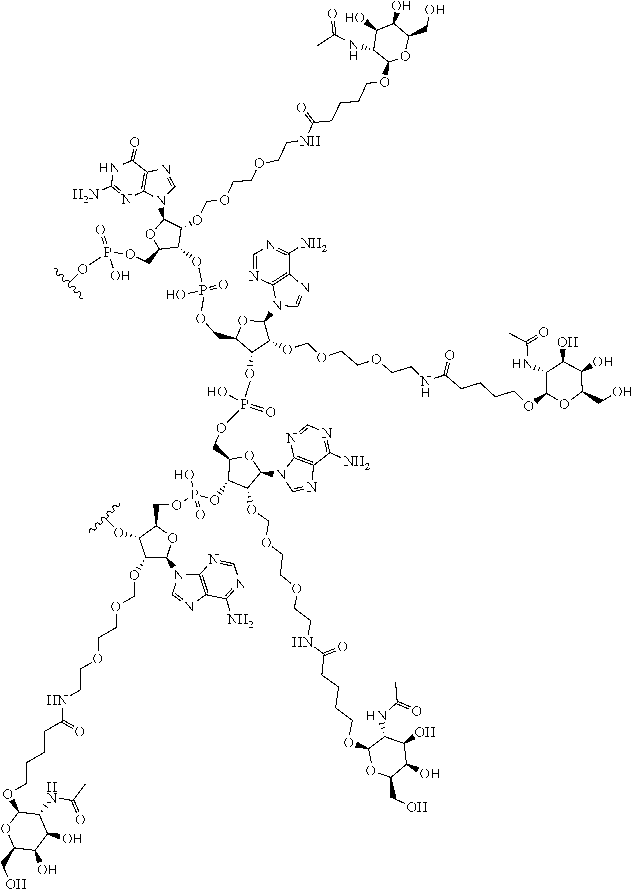 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In some embodiments, an oligonucleotide herein (e.g., an RNAi oligonucleotide) comprises a sense strand having a tetraloop, wherein three (3) GalNAc moieties are conjugated to nucleotides comprising the tetraloop, and wherein each GalNAc moiety is conjugated to one (1) nucleotide. In some embodiments, an oligonucleotide herein (e.g., an RNAi oligonucleotide) comprises a sense strand having a tetraloop comprising GalNAc-conjugated nucleotides, wherein the tetraloop comprises the following structure:

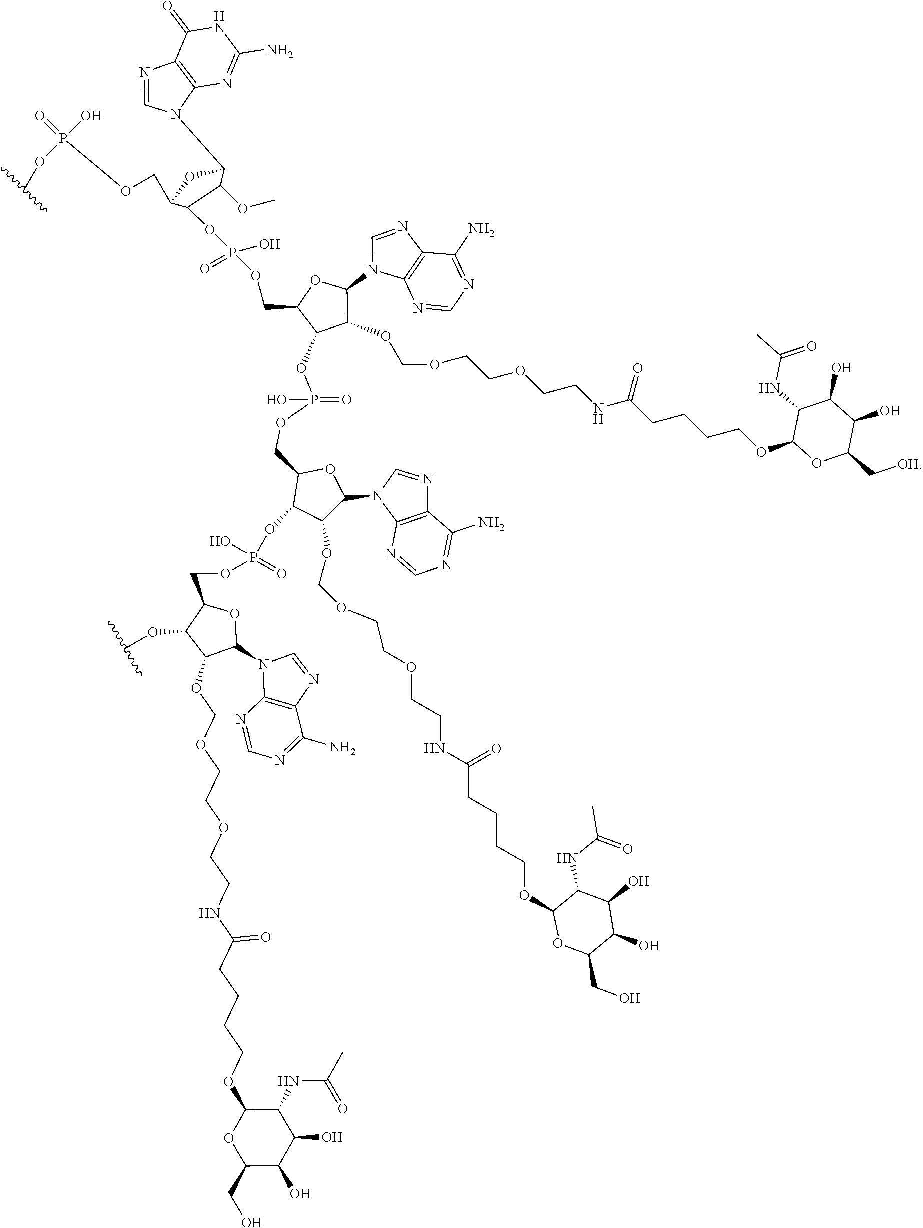

In some embodiments, a duplex extension (e.g., of up to 3, 4, 5, or 6 base pairs in length) is provided between a targeting ligand (e.g., a GalNAc moiety) and an oligonucleotide (e.g., RNAi oligonucleotide). In some embodiments, the duplex extension between a targeting ligand (e.g., a GalNAc moiety) and an oligonucleotide (e.g., RNAi oligonucleotide) is 6 base pairs in length.

### Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions including oligonucleotides (e.g., single-stranded or double-stranded oligonucleotides) to reduce the expression of CFB. Such compositions can be suitably formulated such that when administered to a subject, either into the immediate environment of a target cell or systemically, a sufficient portion of the oligonucleotides enter the cell to reduce CFB expression. Any of a variety of suitable oligonucleotide formulations can be used to deliver oligonucleotides for the reduction of CFB as disclosed herein. In some embodiments, an oligonucleotide, the pharmaceutical composition, the vector, or the cell is formulated in buffer solutions, such as phosphate buffered saline solutions, liposomes, micellar structures, vectors, and capsids.

Formulations as disclosed herein may include an excipient. The excipient may confer to a composition improved stability, improved absorption, improved solubility, and/or therapeutic enhancement of the active ingredient. The excipient may be a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide may be lyophilized for extending its shelf-life and then made into a solution before use (e.g., administration to a subject). Accordingly, an excipient in a composition including any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

The pharmaceutical composition including the oligonucleotide may be formulated to be compatible with its intended route of administration. Examples of routes of administration include parenteral, e.g., subcutaneous, intravenous, intradermal, oral (e.g., inhalation), transdermal (topical), transmucosal, and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). The carrier can be a solvent or dispersion medium containing, for example, water, ethanol, polyol (e.g., glycerol, propylene glycol, liquid polyetheylene glycol, and the like), and suitable mixtures thereof. In many cases, it will be optional to include isotonic agents, for example, sugars, polyalcohols, such as mannitol and sorbitol, and sodium chloride in the composition. Sterile injectable solutions may be prepared by incorporating the oligonucleotides in a required amount in a selected solvent with one or a combination of ingredients enumerated above, as required, followed by filtered sterilization.

In some embodiments, a pharmaceutical composition including the oligonucleotide comprises sterile water (WFI). In some embodiments, a pharmaceutical composition including the oligonucleotide comprises PBS.

In some embodiments, a pharmaceutical composition comprising the oligonucleotide is a preservative-free solution. In some embodiments, the pharmaceutical composition comprises a sterile solution in WFI. In some embodiments, 0.1N NaOH or 0.1N HCl is titrated to adjust the pH of the solution to a target of about 7.2 (e.g., pH 7.2). In some embodiments, the total concentration of the oligonucleotide may be about 160 mg/ml (e.g., 160/mg/mL) as the free acid form. In some embodiments, WFI may be added to bring the pharmaceutical composition to the desired total concentration of oligonucleotide. In some embodiments, the target fill volume is about 1.3 mL into a 2 mL glass vial. In some embodiments, the solution is to be administered to a subject subcutaneously.

In some embodiments, a composition may contain at least about 0.1% of the therapeutic agent (e.g., an oligonucleotide for reducing CFB expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### Pharmaceutical Use

Disclosed herein are methods for delivering to a cell or a subject an effective amount of any one of the oligonucleotides disclosed herein for purposes of reducing expression of CFB in the cell or subject.

The oligonucleotides disclosed herein can be introduced to a cell of a subject with a disease or disorder mediated by complement pathway activation or dysregulation (e.g., activation or dysregulation of CFB) using any appropriate nucleic acid delivery method. For example, the oligonucleotides may be delivered to the cell by injecting a solution containing the oligonucleotides, bombardment by particles covered by the oligonucleotides, exposing the cell or organism to a solution containing the oligonucleotides, or electroporation of cell membranes in the presence of the oligonucleotides.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche), all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

Effective intracellular concentrations of an oligonucleotide disclosed herein may also be achieved via the stable expression of a polynucleotide encoding the oligonucleotide (e.g., by integration into the nuclear or mitochondrial genome of a mammalian cell) or by the temporary expression in a cell contacted with a polynucleotide (e.g., a plasmid or other vector (e.g., a viral vector) encoding the oligonucleotide. Examples of expression vectors are disclosed in, e.g., WO 1994/011026 and are incorporated herein by reference. Expression vectors for use in the compositions and methods described herein contain an oligonucleotide sequence that reduces CFB expression, as well as, e.g., additional sequence elements used for the expression of these agents and/or the integration of these polynucleotide sequences into the genome of a mammalian cell. The expression vector may be a viral vector, a retroviral vector, an adenoviral vector, or an adeno-associated viral vector.

Other methods for delivering oligonucleotides to cells may also be used, such as lipid-mediated carrier transport, chemical-mediated transport, cationic liposome transfection such as calcium phosphate, and vectors including the oligonucleotides. The vectors used for delivery of the oligonucleotides described herein may be viral vectors, such as a retroviral vector (e.g., a lentiviral vector), an adenoviral vector (e.g., Ad5, Ad26, Ad34, Ad35, and Ad48), and an adeno-associated viral vector (AAV) (e.g., AAV1, AAV2, AAV3, AAV4, AAV5, AAV6, AAV7, AAV8, AAV9, and AAV10).

In some examples, an oligonucleotide described herein may be delivered in the form of a transgene that is engineered to express in a cell the oligonucleotides (e.g., its sense and antisense strands). Transgenes may be delivered using a vector, e.g., a viral vector (e.g., adenovirus, retrovirus, vaccinia virus, poxvirus, adeno-associated virus, or herpes simplex virus), as described above or a non-viral vector (e.g., plasmids or synthetic mRNAs). In some embodiments, transgenes can be injected directly into a subject, e.g., at or near the source of action (e.g., within or near the liver) or within the bloodstream.

### CFB Inhibition

Upon administration, the oligonucleotides of the disclosure are capable of binding to and inhibiting the expression of the CFB mRNA. Inhibition of the expression of a CFB gene may be manifested by a reduction of the amount of mRNA expressed by a first cell or group of cells (such cells may be present, for example, in a sample derived (e.g., obtained) from a subject) in which a CFB gene is transcribed and which has or have been treated (e.g., by contacting the cell or cells with an oligonucleotide (e.g., RNAi oligonucleotide) of the disclosure, or by administering an oligonucleotide (e.g., RNAi oligonucleotide) of the disclosure to a subject in which the cells are or were present) such that the expression of a CFB gene is inhibited, as compared to a second cell or group of cells substantially identical to the first cell or group of cells but which has not or have not been so treated (control cell(s) not treated with an oligonucleotide (e.g., RNAi oligonucleotide) or not treated with an oligonucleotide (e.g., RNAi oligonucleotide) targeted to the gene of interest). The level of target mRNA may be measured using techniques well known to one of skill in the art, such as RT-qPCR. The degree of inhibition may be expressed in terms of:

$$\frac{( mRNA in control cells ) - ( mRNA in treated cells )}{( mRNA in control cells )} \times 100 \%$$

A change in the levels of expression of the CFB gene may be assessed in terms of a reduction of a parameter that is functionally linked to CFB gene expression, e.g., CFB protein expression, CFB protein activity, or CFB signaling pathways. CFB gene silencing may be determined in any cell expressing CFB, either endogenous or heterologous from an expression construct, and by any assay known in the art.

The consequences of inhibition of the CFB mRNA can be confirmed by an appropriate assay to evaluate one or more properties of a cell or subject, or by biochemical techniques that evaluate molecules indicative of CFB expression (e.g., RNA, protein). The extent to which an oligonucleotide provided herein reduces levels of expression of CFB is evaluated by comparing expression levels to an appropriate control (e.g., a level of CFB mRNA expression in a cell or population of cells to which an oligonucleotide has not been delivered or to which a negative control has been delivered). An appropriate control level of CFB mRNA expression may be a predetermined level or value, such that a control level need not be measured every time. The predetermined level or value can take a variety of forms including a single cut-off value, such as a median or mean. For example, the predetermined level or value may be at or about a level of 200 μg/mL of CFB protein, which corresponds to a level of CFB protein that is typically found in the serum of healthy subjects.

The level of expression CFB mRNA in a sample may be determined, for example, by detecting a transcribed polynucleotide, or portion thereof, e.g., mRNA. RNA may be extracted from cells using RNA extraction techniques including, for example, using acid phenol/guanidine isothiocyanate extraction RNAZOL™ B; Biogenesis), RNEASY™ RNA preparation kits (Qiagen) or PAXGENE™ (PreAnalytix, Switzerland). The CFB mRNA in a sample may also be determined using real-time PCR (RT-PCR). For example, RNA may be extracted by homogenizing tissue samples in QIAzo Lysis reagent using TissueLyser II (Qiagen) and purifying using MAGMAX® Technology (ThermoFisher Scientific) according to the manufacturer's instructions. High-capacity cDNA reverse transcription kits (ThermoFisher Scientific) may then be used to prepare cDNA. Specific primers and probes for CFB and a housekeep control were used for PCR on a CFX384 Real-Time PCR Detection System (Bio-Rad Laboratories), and the BioRad CFX Maestro Software was used to estimate Ct values; the expression level was calculated in EXCEL® and plotted in Prism (GraphPad). Primers that can be used for RT-PCR include those described in Table 2. Primers having a nucleic acid sequence of any one of SEQ ID NOS: 39-42 may be used to determine the level of CFB in human cells. Likewise, primers having a nucleic acid sequence of SEQ ID NOs: 43 and 44 may be used to determine the level of CFB in monkey cells. Furthermore, primers having a nucleic acid sequence of SEQ ID NOS: 43 and 44 may be used to determine the level of CFB in mouse cells.

TABLE 2
Primers used for RT-PCR

| | | | | SEQ |
|---|---|---|---|---|
| Gene | | | Dye and | ID |
| Target | Primers | Sequence/Probe ID | Quencher | NO: |
| hCFB- | Forward | GACTCGGAAGGAGGTCTACAT | N/A | 39 |
| F2198 | | | | |
| hCFB- | Reverse | CCTCTGAGATGTCCTTGACTTTG | N/A | 40 |
| R2279 | | | | |
| hCFB- | Probe | /56- | FAM/ZEN/ | 69 |
| P2235 | | FAM/AAAGGCAGC/ZEN/TGTGAGAGAGATGCT/ | IABkFQ | |
| | | 3IABkFQ/ | | |
| hCFB- | Forward | AACAGAAGCGGAAGATCGTC | N/A | 41 |
| F1045 | | | | |
| hCFB- | Reverse | TCACACCATAACTTGCCACC | N/A | 42 |
| R1194 | | | | |
| hCFB- | Probe | CCAGCAACTTCACAGGAGCCAAAA | FAM/ZEN/ | 70 |
| P1126 | | | IABkFQ | |
| Monkey | Taqman | Mf02805463_g1 | 5′-FAM | — |
| CFB | Forward | AAT GAA CTG CAG GAC GAG G | N/A | 43 |
| | Reverse | AGG TGA GAT GAC AGG AGA TCC | N/A | 44 |
| | Probe | /5HEX/CAC TGA AGC /ZEN/GGG AAG GGA | 5′-Hex, Zen, 3′- | 72 |
| | | CTG G/3IABkFQ/ | IABkFQ | |
| RhHPRT1 | RhHPRT1- | CTT TCC TTG GTC AGG CAG TAT | N/A | 45 |
| | F583 | | | |
| | RhHPRT1- | CAA CAC TTC GTG GAG TCC TT | N/A | 46 |
| | R642 | | | |
| | RhHPRT1- | /5HEX/CC AAA GAT G/ZEN/G TCA AGG TCG | 5′-HEX, ZEN, 3′ | 74 |
| | R607 | CAA GC/3IABkFQ/ | IABLFQ | |
| RhPPIB | Taqman | Mf02802985_m1 | 5′-VIC-MGB | — |
| CFB | mCFB | CGG AAG GAG GTG TAC ATC AAG | N/A | 47 |
| | F2323 | | | |
| | mCFB | GAG GCA TCT TTG ACC TTC TCA TA | N/A | 48 |
| | R2391 | | | |
| | mCFB | /56-FAM/AG AGA TGC T/ZEN/A CAA AGG CCC | FAM/ZEN/IABkFQ | 76 |
| | P2367 | AAG GC/3IABkFQ/ | | |
| Mm | Forward | CAA ACT TTG CTT TCC CTG GT | N/A | 49 |
| HPRT | | | | |
| F576 | | | | |
| Mm | Reverse | CAA CAA AGT CTG GCC TGT ATC | N/A | 50 |
| HPRT | | | | |
| R664 | | | | |
| Mm | Probe | TGGTTAAGGTTGCAAGCTTGCTGGTG | 5′-Hex, Zen, 3′- | 77 |
| HPRT | | | IABkFQ | |
| P616 | | | | |

Typical assay formats utilizing ribonucleic acid hybridization include nuclear run-on assays, RT-PCR, RNase protection assays, northern blotting, in situ hybridization, and microarray analysis. Circulating mRNA may be detected using methods the described in PCT Publication WO 2012/177906, the entire contents of which are hereby incorporated herein by reference. The level of expression of the gene of interest may also be determined using a nucleic acid probe.

Isolated mRNA can be used in hybridization or amplification assays that include, but are not limited to, Northern or southern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. The mRNA may be immobilized on a solid surface and contacted with a probe, for example by running the isolated mRNA on an agarose gel and transferring the mRNA from the gel to a membrane, such as nitrocellulose. The probe(s) may also be immobilized on a solid surface and the mRNA is contacted with the probe(s), for example, in an AFFYMETRIX® GENECHIP® array. Known mRNA detection methods in the art may be adapted for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known in the art. These detection schemes are useful for the detection of nucleic acid molecules if such molecules are present in very low numbers. In some aspects of the disclosure, the level of expression of a gene of interest (e.g., CFB) is determined by quantitative fluorogenic RT-PCR (i.e., the TAQMAN™ System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads, or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

Using the assays described above, a determination can be made about the effectiveness of treatment with the oligonucleotides described herein based on the amount of CFB mRNA reduction. The reduction in levels of CFB mRNA may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of CFB mRNA or a level of CFB in the subject prior to the treatment. The appropriate control level may be a level of CFB mRNA expression in a cell or population of cells that has not been contacted with an oligonucleotide as described herein. In some embodiments, the effect of delivery of an oligonucleotide to a cell according to a method disclosed herein is assessed after a finite period of time. For example, levels of CFB mRNA may be analyzed in a cell at least 8 hours, 12 hours, 18 hours, 24 hours; or at least 3, 4, 5, 10, 15, 20, 30, 40, 50, 60, 70, or 80 days after introduction of the oligonucleotide into the cell.

Furthermore, inhibition of the CFB gene may result in the inhibition of CFB protein expression which may be manifested by a reduction in the level of the CFB protein that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells.

The consequences of inhibition of the CFB protein expression can be confirmed by an appropriate assay to evaluate one or more properties of a cell or subject, or by biochemical techniques that evaluate molecules indicative of CFB protein expression. The extent to which an oligonucleotide provided herein reduces levels of expression of CFB protein is evaluated by comparing expression levels to an appropriate control (e.g., a level of CFB protein expression in a cell or population of cells to which an oligonucleotide has not been delivered or to which a negative control has been delivered). An appropriate control level of CFB protein expression may be a predetermined level or value, such that a control level need not be measured every time, such as an amount of CFB protein determined to be in the normal range, e.g., about 200 μg/mL in serum. The predetermined level or value can take a variety of forms including a single cut-off value, such as a median or mean.

The level of CFB protein produced by the expression of the CFB gene may be determined using any method known in the art for the measurement of protein levels. Such methods include, for example, electrophoresis, capillary electrophoresis, high performance liquid chromatography (HPLC), liquid chromatography tandem mass spectrometry (LC/MS/MS), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

Using the assays described above, a determination can be made about the effectiveness of treatment with the oligonucleotides described herein based on the amount of CFB protein reduction. The reduction in levels of CFB protein may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of CFB (e.g., about 200 μg/mL). The appropriate control level may be a level of CFB expression in a cell or population of cells that has not been contacted with an oligonucleotide as described herein. The effect of delivery of an oligonucleotide to a cell according to a method disclosed herein may be assessed after a finite period of time. For example, levels of CFB may be analyzed in a cell at least 8 hours, 12 hours, 18 hours, 24 hours; or at least one, two, three, four, five, six, seven, or fourteen days after introduction of the oligonucleotide into the cell. The level of CFB may be determined in order to assess whether re-treatment of the subject is needed. For example, if a level of CFB increases to a pre-treatment level (or a level that is at least about 20% or more (e.g., 30%, 40%, 50%, 60%, 70%, 80%, 90%, or more) of the pre-treatment level), the subject may be in need of re-treatment.

Furthermore, inhibition of the CFB gene using the methods described herein may result in reducing transcription of CFB mRNA in a cell of a subject identified as having a disease mediated by complement pathway activation and dysregulation. Methods provided herein are useful in any appropriate cell type (e.g., a cell that expresses CFB, such as a hepatocyte). In some embodiments, the cell is a primary cell that has been obtained from a subject and that may have undergone a limited number of passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell an effective amount of an oligonucleotide(s) disclosed herein for purposes of reducing expression of CFB solely in hepatocytes.

An effective amount of an oligonucleotide(s) disclosed herein may be determined as the amount of an oligonucleotide(s) that results in a reduction in symptoms of a disease or disorder mediated by complement pathway activation or dysregulation, such as one of the diseases or disorders described herein. The reduction in symptoms of a disease or disorder mediated by complement pathway activation or dysregulation may be a reduction of at least 10%, at least 20%, at least 30%, at least 40%, at least 50, at least 60%, at least 70%, at least 80%, at least 90%, or 100%, e.g., as determined using clinical assessments known to a person of skill in the art. The amount of reduction in symptoms of a disease or disorder mediated by complement pathway activation or dysregulation may be used to determine if subject is in need of being treated again with an RNAi oligonucleotide(s), pharmaceutical composition(s), vector(s), or cell(s) as described herein. Examples of assays to determine reduction in a disease or disorder mediated by complement pathway activation or dysregulation include but are not limited to measuring and/or quantifying circulating CFB protein functional assays (e.g., Weislab assay and hemolytic assay). Quantitation of CFB deposition may be performed via IHC or immunofluorescence or via specific disease biomarkers.

Furthermore, an oligonucleotide described herein that includes both a sense strand and an antisense strand as a duplex oligonucleotide may be introduced to a cell of a subject using any appropriate nucleic acid delivery. The duplex oligonucleotide may be delivered to the cell by injecting a solution containing the oligonucleotide, bombardment by particles covered by the oligonucleotide, exposing the cell or organism to a solution containing the oligonucleotide, or electroporation of cell membranes in the presence of the oligonucleotide. The duplex oligonucleotides may also be delivered to the cells using lipid-mediated carrier transport, chemical-mediated transport, cationic liposome transfection such as calcium phosphate, and vectors encoding the nucleic acids of the single-strand oligonucleotide. The vectors used for delivery of the duplex oligonucleotide may be viral vectors, such as a retroviral vector (e.g., a lentiviral vector), an adenoviral vector (e.g., Ad5, Ad26, Ad34, Ad35, and Ad48), and an adeno-associated viral vector (AAV) (e.g., AAV1, AAV2, AAV3, AAV4, AAV5, AAV6, AAV7, AAV8, and AAV9)

### Treatment Methods

Also, disclosed herein are methods for the treatment of diseases mediated by complement pathway activation or dysregulation, including, e.g., one or more of the diseases associated with complement pathway activation or dysregulation disclosed herein, in a subject by administration of the composition described herein (e.g., an oligonucleotide, a vector encoding an oligonucleotide, a cell containing the vector, and a pharmaceutical composition). The method may include the treatment of diseases mediated by complement pathway activation or dysregulation in a subject by administration of a pharmaceutically acceptable salt (e.g., a sodium salt) of the RNAi oligonucleotide described herein. The methods described herein typically involve administering to a subject an effective amount of an oligonucleotide, or pharmaceutically acceptable salt thereof, that is, an amount capable of producing a desirable therapeutic result (e.g., knockdown of CFB expression). A therapeutically acceptable amount may be an amount that is capable of treating a disease or disorder mediated by complement pathway activation or dysregulation (e.g., activation or dysregulation of CFB). The appropriate dosage for any one subject will depend on certain factors, including the subject's size, body surface area, age, the particular composition to be administered, the active ingredient(s) in the composition, time and route of administration, general health, and other drugs being administered concurrently. Such treatments could be used, for example, to slow, halt, or prevent any type of disease or disorder mediated by complement pathway activation or dysregulation and may be administered either prophylactically or therapeutically. Administration of a prophylactic agent can occur prior to the detection of, or the manifestation of symptoms characteristic of the disease or disorder mediated by complement pathway activation or dysregulation, such that the disease or disorder is prevented or, alternatively, delayed in its progression. Subjects at risk for a disease or disorder mediated by complement pathway activation or dysregulation can be identified by, for example, one or a combination of diagnostic or prognostic assays known in the art.

The compositions disclosed herein may be administered to a subject using any standard method. For example, any one of the compositions disclosed herein may be administered enterally (e.g., orally, by gastric feeding tube, by duodenal feeding tube, via gastrostomy, or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously. The most suitable route for administration in any given case will depend on the particular composition administered, the subject, the particular disease or disorder mediated by complement pathway activation or dysregulation being treated, pharmaceutical formulation methods, administration methods (e.g., administration time and administration route), the subject's age, body weight, sex, severity of the diseases being treated, the subject's diet, and the subject's excretion rate.

The subject suffering from the disease or disorder mediated by complement pathway activation or dysregulation may be administered the oligonucleotides described herein, for example, annually (e.g., once every 12 months), semi-annually (e.g., once every six months), quarterly (e.g., once every three months), bi-monthly (e.g., once every two months), monthly, or weekly. In other instances, the oligonucleotides may be administered one or more times every one, two, or three weeks, one or more times per month, every other month, one or more times per three months, one or more times quarterly, one or more times every six months, or one or more times per year. In certain embodiments, the oligonucleotides may be administered daily.

The subject to be treated for a disease or disorder mediated by complement pathway activation or dysregulation may be a human or non-human primate or another mammalian subject. Other exemplary subjects that may be treated with the oligonucleotides described herein include domesticated animals such as dogs and cats; livestock such as horses, cattle, pigs, sheep, goats, and chickens; and animals such as mice, rats, guinea pigs, and hamsters.

### Dosages

A dosage of the composition of the disclosure (e.g., a composition including an RNAi oligonucleotide, or pharmaceutically acceptable salt thereof, as described herein) can vary depending on many factors, such as the pharmacodynamic properties of the compound, the mode of administration, the age, health, and weight of the recipient, the nature and extent of the symptoms, the frequency of the treatment and/or the type of concurrent treatment, if any, and the clearance rate of the compound in the subject to be treated. One of skill in the art can determine the appropriate dosage based on the above factors.

The oligonucleotides of the disclosure, or pharmaceutically acceptable salts thereof, may be administered in an amount and for a time effective to result in one or more of (e.g., 2 or more, 3 or more, 4 or more of): (a) decreased expression of CFB protein in a cell of the subject, (b) reduced transcription of CFB in the cell of the subject, (c) reduced level of CFB protein in the cell of the subject, (d) reduced activity of the CFB protein the in cell of the subject; and/or (e) reduction in one or more symptoms of a disease or disorder mediated by complement pathway activation or dysregulation.

Accordingly, the disclosure relates to a method for treating a disease or disorder mediated by complement pathway activation or dysregulation in a subject in need thereof, in which the method includes administering an effective amount of an oligonucleotide described herein that binds specifically to CFB mRNA and inhibits expression of CFB protein in the subject. For example, the disclosure provides a method of treating a disease or disorder mediated by alternative complement pathway dysregulation in a subject in need thereof including administering to the subject a therapeutically effective amount of an oligonucleotide, pharmaceutical composition, vector, or cell disclosed herein.

The disease or disorder mediated by complement pathway activation or dysregulation to be treated utilizing the disclosed methods and compositions may be, e.g., cutaneous disorders, neurological disorders, nephrology disorders, acute care, rheumatic disorders, pulmonary disorders, dermatological disorders, hematologic disorders, and ophthalmic disorders.

The treatment of diseases mediated by complement pathway activation or dysregulation can be accomplished by administration of an oligonucleotide, or pharmaceutically acceptable salt thereof, that inhibits the expression and/or translation of CFB mRNA (e.g., the expression of CFB protein), such as those described herein.

The disclosed compositions can be administered in amounts determined to be appropriate by those of skill in the art. In some embodiments, the oligonucleotide, or pharmaceutically acceptable salt thereof, described herein may be administered initially in a suitable dosage that may be adjusted as required, depending on the clinical response.

In some instances, the oligonucleotide, or pharmaceutically acceptable salt thereof, is administered at a dose of 0.01-100 mg/kg (e.g., 0.01-1 mg/kg, 1-5 mg/kg, 5-20 mg/kg, 20-50 mg/kg, 50-100 mg/kg) of bodyweight of a subject. In certain instances, the oligonucleotide is administered at a concentration of 0.01 mg/kg-50 mg/kg (e.g., 0.01-1 mg/kg, 1-5 mg/kg, 5-10 mg/kg, 10-20 mg/kg, 20-30 mg/kg, 30-40 mg/kg, 40-50 mg/kg) bodyweight of the subject. In other instances, the oligonucleotide is administered at a concentration of 0.01 mg/kg-20 mg/kg (e.g., 0.01-1 mg/kg, 1-5 mg/kg, 5-10 mg/kg, 10-15 mg/kg, 15-20 mg/kg) bodyweight of the subject. In other instances, the oligonucleotide is administered at a concentration of 0.01 mg/kg-15 mg/kg (e.g., 0.01-1 mg/kg, 1-2 mg/kg, 2-5 mg/kg, 5-8 mg/kg, 8-10 mg/kg, 10-12 mg/kg, 12-15 mg/kg) bodyweight of the subject. In other instances, the oligonucleotide is administered at a concentration of 0.01 mg/kg-10 mg/kg (e.g., 0.01-1 mf/kg, 1-2 mg/kg, 2-5 mg/kg, 5-8 mg/kg, 8-10 mg/kg) bodyweight of the subject. In other instances, the oligonucleotide is administered at a concentration of 0.01 mg/kg-5 mg/kg (e.g., 0.01-1 mg/kg, 1-2 mg/kg, 2-3 mg/kg, 3-4 mg/kg, 4-5 mg/kg) bodyweight of the subject. In other instances, the oligonucleotide is administered at a concentration of 0.1 mg/kg-20 mg/kg (0.1-1 mg/kg, 1-5 mg/kg, 5-10 mg/kg, 10-15 mg/kg, and 15-20 mg/kg) bodyweight of the subject. In other instances, the oligonucleotide is administered at a concentration of 0.1 mg/kg-10 mg/kg (e.g., 0.1-1 mg/kg, 1-2 mg/kg, 2-5 mg/kg, 5-7 mg/kg, and 7-10 mg/kg) bodyweight of the subject. In other instances, the oligonucleotide is administered at a concentration of 0.1 mg/kg-5 mg/kg (e.g., 0.1-1 mg/kg, 2-3 mg/kg, 3-4 mg/kg, and 4-5 mg/kg) bodyweight of the subject. In other instances, the oligonucleotide is administered at a concentration of 1 mg/kg-50 mg/kg (e.g., 1-10 mg/kg, 10-20 mg/kg, 20-30 mg/kg, 30-40 mg/kg, and 40-50 mg/kg) bodyweight of the subject. In other instances, the oligonucleotide is administered at a concentration of 1 mg/kg-20 mg/kg (e.g., 1-5 mg/kg, 5-10 mg/kg, 10-15 mg/kg and 15-20 mg/kg) bodyweight of the subject. In other instances, the oligonucleotide is administered at a concentration of 1 mg/kg-10 mg/kg (e.g., 1-2 mg/kg, 2-5 mg/kg, 5-7 mg/kg, and 7-10 mg/kg) bodyweight of the subject. In other instances, the oligonucleotide is administered at a concentration of 1 mg/kg-5 mg/kg (e.g., 1-2 mg/kg, 2-3 mg/kg, 3-4 mg/kg, and 4-5 mg/kg) bodyweight of the subject. In other instances, the oligonucleotide is administered at a concentration of 30 mg/kg-300 mg/kg (e.g., 30-200 mg/kg, 30-100 mg/kg, 30-50 mg/kg, 50-300 mg/kg, 100-300 mg/kg, 200-300 mg/kg, and 250-300 mg/kg).

In certain embodiments, the oligonucleotide, or pharmaceutically acceptable salt thereof, is administered at a dose of less than 10 mg/kg (e.g., 9 mg/kg or less, 8 mg/kg or less, 7 mg/kg or less, 6 mg/kg or less, 5 mg/kg or less, 4 mg/kg or less, 3 mg/kg or less, 2 mg/kg or less, 1 mg/kg or less) bodyweight of the subject. In other embodiments, the oligonucleotide is administered at a dose of about 10 mg/kg or less. In another embodiment, the oligonucleotide is administered at a dose of about 9 mg/kg or less (e.g., 8.9 mg/kg, 8 mg/kg, 7 mg/kg, 5 mg/kg, 3 mg/kg, and 1 mg/kg or less). In other embodiments, the oligonucleotide is administered at a dose of about 8 mg/kg or less (e.g., 7.9 mg/kg, 7 mg/kg, 5 mg/kg, 3 mg/kg, and 1 mg/kg or less). In another embodiment, the oligonucleotide is administered at a dose of about 7 mg/kg or less (e.g., 6.9 mg/kg, 6 mg/kg, 4 mg/kg, 2 mg/kg, and 1 mg/kg or less). In another embodiment, the oligonucleotide (e.g., RNAi oligonucleotide) is administered at a dose of about 6 mg/kg or less (e.g., 5.9 mg/kg, 5 mg/kg, 3 mg/kg, and 1 mg/kg or less). In another embodiment, the oligonucleotide is administered at a dose of about 5 mg/kg or less (e.g., 4.9 mg/kg, 4 mg/kg, 3 mg/kg, 2 mg/kg, and 1 mg/kg or less). In another embodiment, the oligonucleotide is administered at a dose of about 4 mg/kg or less (e.g., 3.9 mg/kg, 3 mg/kg, 2 mg/kg, and 1 mg/kg or less). In another embodiment, the oligonucleotide is administered at a dose of about 3 mg/kg or less (e.g., 2.9 mg/kg, 2.5 mg/kg, 2 mg/kg, 1 mg/kg or less). In another embodiment, the oligonucleotide is administered at a dose of about 2 mg/kg or less (e.g., 1.9 mg/kg, 1.5 mg/kg, 1 mg/kg, and 0.5 mg/kg or less). In another embodiment, the oligonucleotide is administered at a dose of about 1 mg/kg or less (e.g., 0.9 mg/kg, 0.8 mg/kg, 0.7 mg/kg, 0.6 mg/kg, 0.5 mg/kg, 0.4 mg/kg, 0.3 mg/kg, 0.2 mg/kg, and 0.1 mg/kg or less).

In another embodiment, the oligonucleotide, or pharmaceutically acceptable salt thereof, is administered at a dose of about 0.1-10 mg/kg, about 0.2-10 mg/kg, about 0.3-10 mg/kg, about 0.4-10 mg/kg, about 0.5-10 mg/kg, about 1-10 mg/kg, about 2-10 mg/kg, about 3-10 mg/kg, about 4-10 mg/kg, about 5-10 mg/kg, about 6-10 mg/kg, about 7-10 mg/kg, about 8-10 mg/kg, or about 9 mg/kg of bodyweight of a subject.

In other instances, the dosage of a composition (e.g., a composition including an oligonucleotide described herein) is a prophylactically or a therapeutically effective amount. In some cases, a viral vector (e.g., an rAAV vector) including the oligonucleotide described herein is administered at a dose of 10⁵, 10⁶, 10⁷, 10⁸, 10⁹, 10¹⁰, 10¹¹, 10¹², 10¹³, 10¹⁴, or 10¹⁵ genome copies (GC) per subject. In some embodiments the viral vector (e.g., rAAV vector) is administered at a dose of 10⁵, 10⁶, 10⁷, 10⁸, 10⁹, 10¹⁰, 10¹¹, 10¹², 10¹³, or 10¹⁴ GC/kg (total weight of the subject). In other instances, the oligonucleotide is administered in a dosage between 0.1 mg/kg to about 150 mg/kg (e.g., 0.5 mg/kg, 1 mg/kg, 5 mg/kg, 10 mg/kg, 20 mg/kg, 50 mg/kg, 75 mg/kg, 100 mg/kg, 150 mg/kg).

Optionally, the disclosed oligonucleotides may be administered as part of a pharmaceutically acceptable composition suitable for delivery to a subject, as is described herein. The disclosed agents are included within these compositions in amounts sufficient to provide a desired dosage and/or elicit a therapeutically beneficial effect, as can be readily determined by those of skill in the art.

The disclosed compositions described herein may be administered in an amount (e.g., an effective amount) and for a time sufficient to treat the subject or to effect one of the outcomes described above (e.g., a reduction in one or more symptoms of disease in the subject). The disclosed compositions may be administered once or more than once. The disclosed compositions may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months, or more following administration of a composition of the disclosure depending on the composition and the route of administration used for treatment. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated (e.g., for the life of the subject). For example, a subject diagnosed with PNH and treated with a composition disclosed herein may be given one or more (e.g., 1, 2, 3, 4, 5, 6, 7, 8, 9, 10, or more) additional treatments if initial or subsequent rounds of treatment do not elicit a therapeutic benefit including reduction of any one of the symptoms associated with PNH, such as fatigue, weakness, shortness of breath, bruising or bleeding easily, recurring infections, severe headache, blood clots, and difficulty controlling bleeding, or a reduction in the levels of CFB mRNA or CFB protein levels in the cells or serum of the subject.

### Kits

The disclosure also features kits including (a) a pharmaceutical composition including an oligonucleotide (e.g., RNAi oligonucleotide) agent, or pharmaceutically acceptable salt thereof, that reduces the level and/or activity of CFB in a cell or subject described herein and, optionally, a pharmaceutically acceptable carrier, excipient, or diluent. The kit may contain a vector encoding an oligonucleotide(s) (e.g., an RNAi oligonucleotide(s)) described herein or a cell including a vector encoding an oligonucleotide(s) (e.g., an RNAi oligonucleotide(s)) described herein. The kit may also include a package insert with instructions to perform any of the methods described herein. In some embodiments, the kit includes (a) a pharmaceutical composition including an oligonucleotide (e.g., RNAi oligonucleotide) agent that reduces the level and/or activity of CFB in a cell or subject described herein, (b) an additional therapeutic agent, and (c) a package insert with instructions to perform any of the methods described herein.

### EXAMPLES

The following examples are intended as illustration only, are not meant to limit the disclosure in any way.

### Example 1: Preparation of RNAi Oligonucleotides

### Oligonucleotide Synthesis and Purification

The RNAi oligonucleotides described in this Example and the following Examples were chemically synthesized using methods described herein. Generally, RNAi oligonucleotides were synthesized using solid phase oligonucleotide synthesis methods as described for 19-23mer siRNAs (see, e.g., Scaringe et al. (1990) Nucleic Acids Res. 18:5433-5441 and Usman et al. (1987) J. Am. Chem. Soc. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) Cold Spring Harb Perspect Biol. 9 (1): a023812; Beaucage S. L., Caruthers M. H. Studies on Nucleotide Chemistry V: Deoxynucleoside Phosphoramidites—A New Class of Key Intermediates for Deoxypolynucleotide Synthesis. Tetrahedron Lett. 22:1859-1862, 1981; doi: 10.1016/S0040-4039 (01) 90461-7).

RNAi oligonucleotides having a 19mer core sequence were formatted into constructs having a 25mer sense strand and a 27mer antisense strand to allow for processing by the RNAi machinery. The 19mer core sequence was complementary to a region in the CFB mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) Methods Mol. Biol. 20:81-114; Wincott et al. (1995) Nucleic Acids Res. 23:2677-2684). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A: B to 52:48 Buffers A: B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DET Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 M concentration) in duplex buffer consisting of 100 mM potassium acetate, 30 mM HEPES, pH 7.5. Complementary sense and antisense strands were mixed in equal molar amounts to yield a final solution of, for example, 50 M duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

### Example 2: Generation of CFB-Targeting RNAi Oligonucleotides

Identification of CFB mRNA Target Sequences

Complement Factor B (CFB) is a protein involved in the alternative pathway of complement. To generate RNAi oligonucleotide inhibitors of CFB expression, a computer-based algorithm was used to computationally identify CFB mRNA target sequences suitable for assaying inhibition of CFB expression by the RNAi pathway. Over 300 RNAi oligonucleotides guide (antisense) strand sequences, each having a region of complementarity to a suitable CFB target sequence of human CFB mRNA (see Table 3), were prepared and assayed in vitro for CFB expression inhibition. From these RNAi oligonucleotides, a subset of nine (see Table 4) were selected for further study. The subset of nine guide sequences identified by the algorithm were also complementary to the corresponding CFB target sequence of monkey CFB mRNA (SEQ ID NO: 51; Table 3). CFB RNAi oligonucleotides comprising a region of complementarity to homologous CFB mRNA target sequences with nucleotide sequence similarity are predicted to have the ability to target homologous CFB mRNAs.

TABLE 3
Sequences of Human and Monkey CFB mRNA

| Species | Ref Seq # | SEQ ID NO |
|---|---|---|
| Human (Hs) | NM_001710 | 12 |
| Cynomolgus monkey (Mf) | XM_005553440 | 51 |

### Example 3: Identification of RNAi Oligonucleotides to Inhibit CFB Expression In Vitro

RNAi oligonucleotides (formatted as DsiRNA oligonucleotides) designed to inhibit CFB expression were individually evaluated in vitro using a cell-based assay. The methods used to prepare the oligonucleotides are described in Example 1. The methods used to design and create the CFB mRNA target sequences are described in Example 2.

### In Vitro Cell-Based Assays

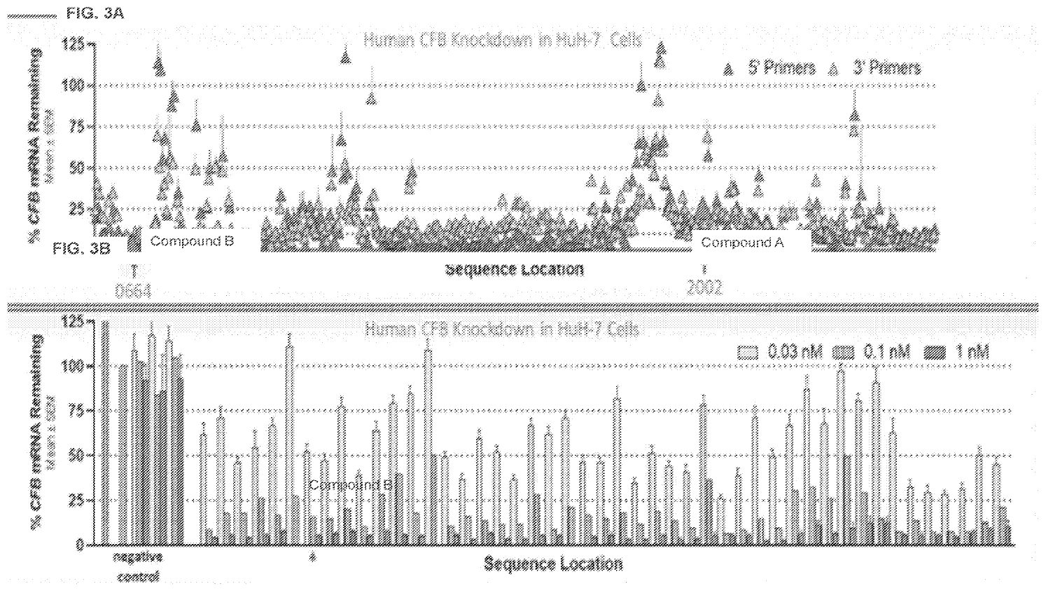

The ability of each of the RNAi oligonucleotides generated to reduce CFB mRNA was measured using in vitro cell-based assays. Briefly, human hepatocyte (Huh7) cells expressing endogenous human CFB gene were transfected with each of the RNAi oligonucleotides at 1 nM in separate wells of a multi-well cell-culture plate (Compounds A-I). Cells were maintained for 24 hours following transfection with the modified RNAi oligonucleotides, and then the amount of remaining CFB mRNA from the transfected cells was determined using TAQMAN®-based qPCR assays. Two qPCR assays, a 3′ assay and a 5′ assay, were used to determine CFB mRNA levels as measured using PCR probes conjugated to 6-carboxy-fluorescein (FAM) and normalized to the HPRT housekeeping gene. Each primer pair was assayed for % remaining CFB mRNA as shown in FIG. 3A. RNAi oligonucleotides resulting in less than or equal to 8% CFB mRNA remaining in the RNAi oligonucleotide-transfected cells when compared to mock-transfected cells were considered RNAi oligonucleotide “hits”.

A dose response study was performed by transfecting HuH-7 human liver cells expressing endogenous CFB with the RNAi oligonucleotides at three different concentrations (0.03 nM, 0.1 nM, and 1 nM; see FIG. 3B) as indicated in separate wells of a multi-well cell-culture plate. Cells were maintained for 24 hr following transfection, and then levels of remaining CFB mRNA from the transfected cells was determined using TAQMAN®-based qPCR assays. Two qPCR assays, a 3′ assay and a 5′ assay, were used to determine mRNA levels as measured by HEX and FAM probes, respectively. The nine RNAi oligonucleotides (Compounds A-I) were further tested in in vivo screening assays.

Taken together, these results show that RNAi oligonucleotides designed to target human CFB mRNA inhibit CFB expression in cells, as determined by a reduced amount of CFB mRNA in RNAi oligonucleotide-transfected cells relative to control cells. These results demonstrate that the nucleotide sequences comprising the RNAi oligonucleotides are useful for generating RNAi oligonucleotides to inhibit CFB expression. Further, these results demonstrate that multiple CFB mRNA target sequences are suitable for the RNAi-mediated inhibition of CFB expression (Table 4).

TABLE 4
Analysis of CFB mRNA in Huh7 cells

| | SEQ ID | Sense | SEQ ID | Antisense | | | | |
|---|---|---|---|---|---|---|---|---|
| | NO | Strand with | NO | Strand with | | | | |
| | (Sense | Modifications | (Antisense | Modifications | CFB-5′ Assay | | CFB-3′ Assay | |
| Compound | Strand) | SEQ ID NO: | Strand) | SEQ ID NO: | % remaining | SEM | % remaining | SEM |
| A | 1 | 66 | 3 | 67 | 24.8 | 3.5 | 23.0 | 3.9 |
| B | 4 | 37 | 6 | 38 | 1.1 | 0.3 | 6.2 | 1.0 |
| C | 17 | 52 | 18 | 53 | 2.3 | 0.2 | 6.9 | 0.8 |
| D | 19 | 54 | 20 | 55 | 2.7 | 0.4 | 7.9 | 1.7 |
| E | 21 | 56 | 22 | 57 | 4.4 | 1.3 | 3.2 | 1.3 |
| F | 23 | 58 | 24 | 59 | 1.3 | 0.5 | 7.3 | 0.7 |
| G | 25 | 60 | 26 | 61 | 0.3 | 0.2 | 5.9 | 2.2 |
| H | 27 | 62 | 28 | 63 | 6.5 | 0.3 | 5.6 | 0.8 |
| I | 29 | 64 | 30 | 65 | 6.2 | 0.8 | 6.4 | 1.1 |

### Example 4: Screening of RNAi Oligonucleotides in Mice Expressing Human CFB cDNA (HDI Mice)

The in vitro screening assay in Example 3 validated the ability of CFB-targeting oligonucleotides to knock-down target mRNA. To confirm the ability of the RNAi oligonucleotides to knockdown CFB in vivo, an HDI mouse model was used.

The oligonucleotides in Table 4 were evaluated in mice engineered to transiently express human CFB mRNA in hepatocytes of the mouse liver. Briefly, 6-8-week-old female CD-1 mice (n=4-5) were subcutaneously administered the indicated RNAi oligonucleotides at a dose of 0.25 mg/kg, 0.3 mg/kg, 0.5 mg/kg, 1 mg/kg, or 3 mg/kg formulated in PBS. A control group of mice (n=5) were administered only PBS. Three days later (72 hours), the mice were hydrodynamically injected (HDI) with a DNA plasmid encoding the full human CFB gene (SEQ ID NO: 12) (25 ug) under control of a ubiquitous cytomegalovirus (CMV) promoter sequence. One day after introducing the DNA plasmid, liver samples from HDI mice were collected. Total RNA derived from these HDI mice were subjected to qRT-PCR analysis to determine CFB mRNA levels as described in Example 3. mRNA levels were measured for human mRNA. The values were normalized for transfection efficiency using the NeoR gene included on the DNA plasmid.

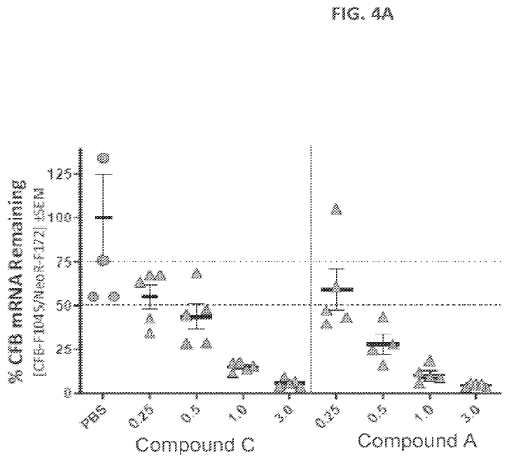

The results in FIGS. 4A-4C demonstrate that the RNAi oligonucleotides designed to target human CFB mRNA inhibited human CFB mRNA expression in HDI mice, as determined by a reduction in the amount of human CFB mRNA expression in liver samples from HDI mice treated with RNAi oligonucleotides relative to control HDI mice treated with only PBS. All the RNAi oligonucleotides tested were able to reduce CFB expression. Overall, the HDI study identified a number of potential RNAi oligonucleotides for inhibiting CFB expression in liver.

### Example 5: Screening of RNAi Oligonucleotides in Cynomolgus Macaques

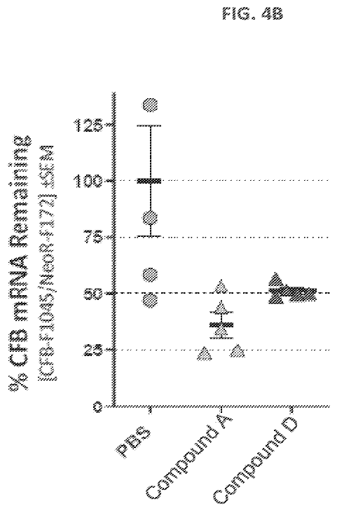

All Compounds A to I, as described in Example 4, which were pre-selected during the mouse screening, were tested in cynomolgus macaques (NHP) for duration of CFB mRNA silencing after a single subcutaneous administration of the Compounds A-I at 4 mg/kg. Liver biopsy of all tested animals (n=5/Compound) were collected before dosing and on Day 28 and Day 56 post injection. As demonstrated in FIG. 5, there was at least 50% reduction of liver CFB mRNA levels for most Compounds tested in comparison to normalized baseline levels and time-matched PBS controls as determined by RT-qPCR. Two lead Compounds (Compounds A and B) were selected based on the knockdown level of CFB mRNA in the liver of cynomolgus macaques post single administration for testing in a multidose study.

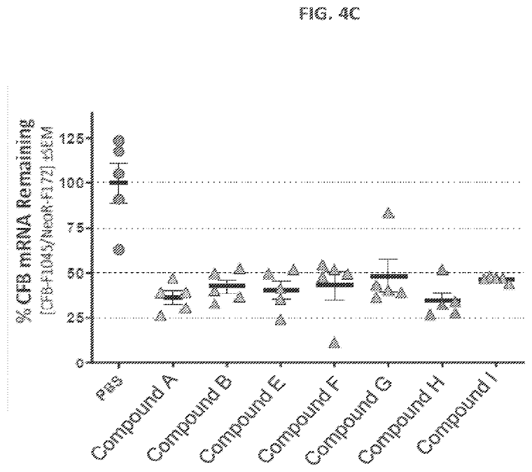

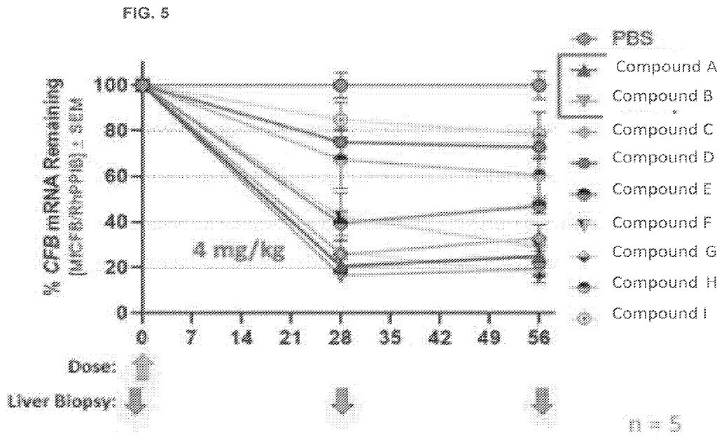

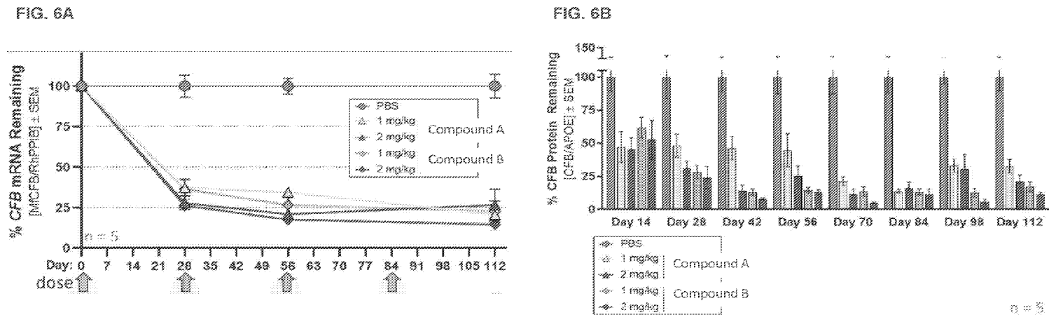

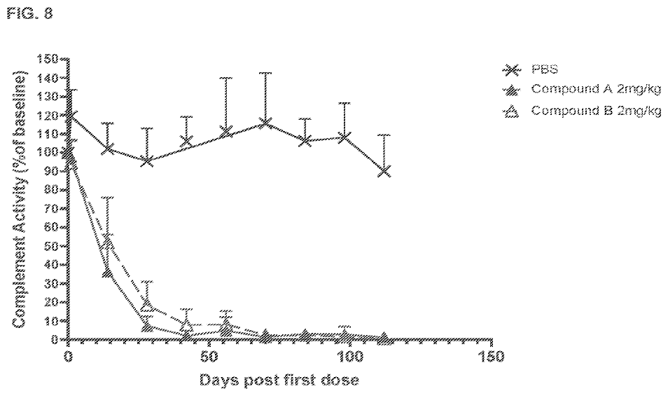

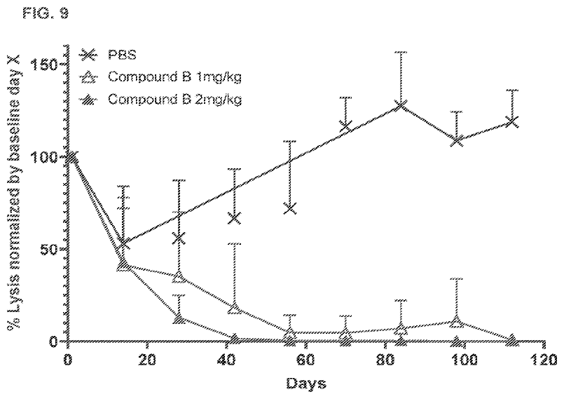

Compounds A and B were selected from the single dose study for further evaluation in a multiple dose NHP study. Cynomolgus macaques were dosed subcutaneously with 1 mg/kg or 2 mg/kg on day 0, day 28, day 56, and day 84 for a total of 4 doses. Liver biopsies were collected pre-dosing and on Days 28, 56, and 112 post initial treatment for evaluation of liver CFB mRNA levels by RT-qPCR (FIG. 6A). Serum samples were collected on pre-dosing, Day 14. 28, 42, 56, 70, 84, 98, and 112 post initial dose for evaluation of CFB protein levels by immunoblot (FIG. 6B), complement activity by WIESLAB® AP assay (FIG. 8), and by hemolysis of rabbit erythrocytes (FIG. 9; only Compound B was tested in the hemolysis assay). PBS-treated animals were used as control from CFB liver mRNA, CFB serum protein, and functional assays. Multiple treatment of cynomolgus macaques with Compound A or B led to a sustained duration of CFB mRNA silencing in the liver, significant reduction of circulating CFB in serum, a >95% reduction of alternative pathway complement activity, and complete inhibition of lysis of rabbit erythrocytes in a hemolytic assay after multiple administrations of Compounds A and B, as depicted in FIGS. 6A, 6B, 8, and 9, respectively.

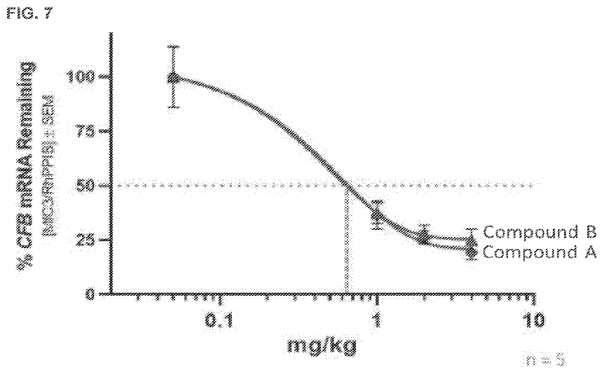

The potency of Compounds A and B was calculated by combining Day 28 results for both single and multidose NHP studies. The approximate ED₅₀ for Compound A (0.65 mg/kg) and Compound B (0.65 mg/kg) was calculated from a dose-response curve generated for both Compounds (FIG. 7).

### Example 6: Pharmacokinetic and Pharmacodynamic Study of Compound J on CFB Expression in CD-1 Mice

CD-1 mice were treated with Compound J to assess the percent of CFB mRNA knockdown in the livers of the mice and the amount of CFB protein in serum of the mice as a result of Compound J administration. Compound J is an RNAi oligonucleotide that targets mouse CFB expression, which acts as a surrogate for the RNAi oligonucleotides which target human CFB expression, such as Compound A and Compound B. The percent knockdown of the liver CFB mRNA as a result of Compound J administration was measured using RT-qPCR. The amount of CFB in serum was qualitatively measured by immunoblot. The mice received a single, subcutaneous dose of Compound J at 0.25 mg/kg, 0.5 mg/kg, or 3 mg/kg.

The single administration of Compound J showed a dose-dependent liver CFB mRNA knockdown percentage, with greater than 90% reduction of CFB mRNA in the liver from animals that received 3 mg/kg dose (n=5 mice/timepoint). The nadir of mRNA knockdown was 3-21 days after 3 mg/kg dose as shown in FIG. 10A. The percentage of CFB protein in the serum of CD-1 mice was measured over the course of the study and was correspondingly suppressed (FIG. 10B).

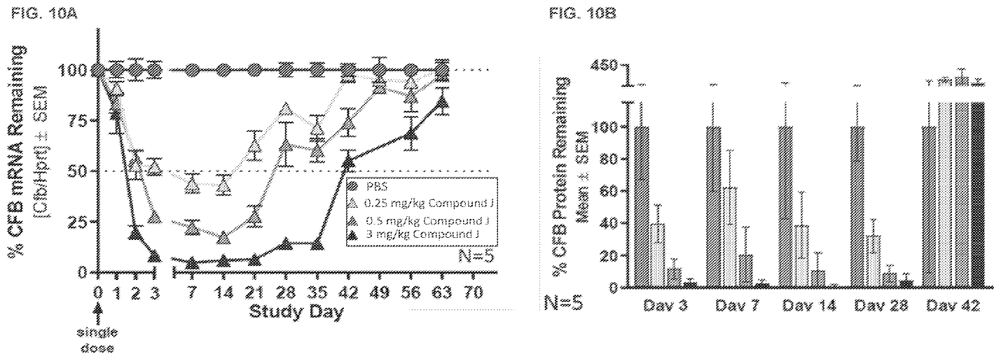

The amount of Compound J in plasma, spleen, liver, and kidney tissues of CD-1 mice, which were administered a single, subcutaneous dose of 3 mg/kg of Compound J, was measured using stem loop-qPCR over a period of 672 hours after receiving the dose (FIG. 11). Pharmacokinetic analysis indicated that the highest exposure of Compound J was in liver, followed by spleen, kidney and plasma (FIG. 11).

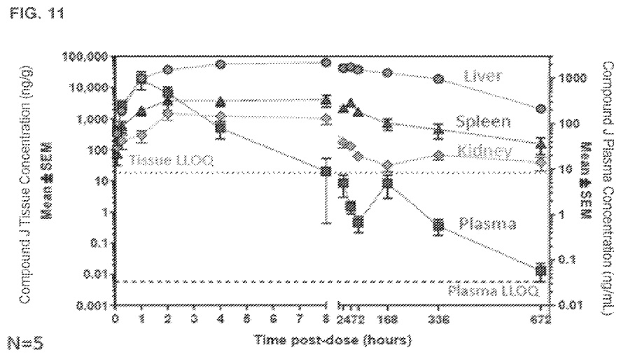

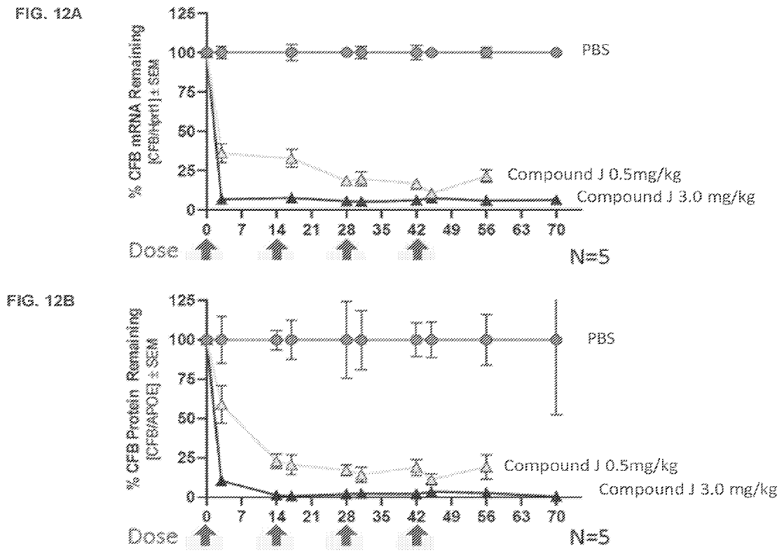

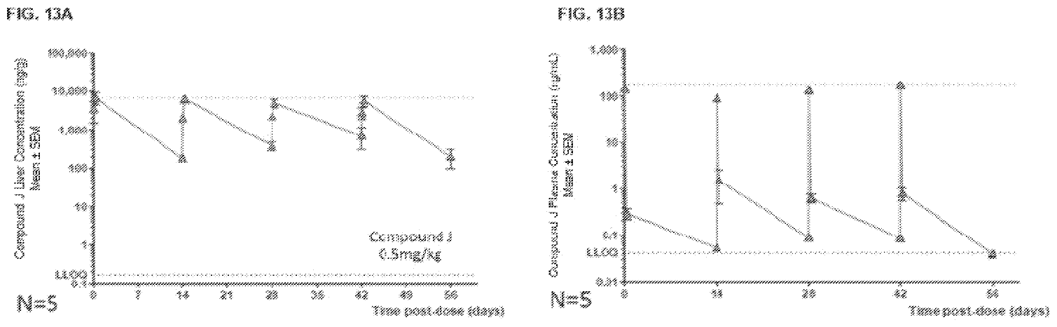

The percentage of liver CFB mRNA was also measured using RT-qPCR and the amount of CFB protein in serum was qualitatively assessed by immunoblot over a 70-day period, where CD-1 mice received four doses of either 0.5 mg/kg or 3 mg/kg of Compound J on days 0, 14, 28, and 42 as shown in FIGS. 12A and 12B, respectively. Liver biopsies and serum collections were performed on day 3, 14, 17, 28, 31, 42, 45, 56, and 70 after the initial dose for animals administered a high dose (3 mg/kg) of Compound J, and at the same time points (except for a collection at day 63 rather than at day 70) for animals administered a low dose (0.5 mg/kg) of Compound J. The liver and plasma concentration of Compound J after 4 doses of 0.5 mg/kg was analyzed from the liver biopsies and plasma samples using Stem Loop qPCR (SL-qPCR) as shown in FIGS. 13A and 13B, respectively. PBS-treated CD-1 mice were used as control for both CFB liver mRNA and CFB serum protein levels.

This multidose study showed that Compound J (a murine surrogate) showed a dose-dependent knockdown of liver CFB mRNA that was sustained over the course of 70 days. The reduction of circulating CFB protein levels corresponded to the reduction of CFB mRNA observed in the liver. Additionally, plasma and liver concentrations of Compound J from dosed animals showed no accumulation of Compound J with biweekly dosing (0.5 mg/kg) (see FIGS. 13A and 13B, respectively).

Further, a single dose Absorption, Distribution, Metabolism, and Excretion (ADME) study in male CD-1 mice was conducted, and the pharmacokinetics of Compound B was characterized following the administration of a single 3, 10, or 100 mg/kg subcutaneous or 3 mg/kg intravenous dose. Following a single subcutaneous administration plasma Compound B concentration reached Tₘₐₓ at 1 hour for all three subcutaneous dose groups and was followed by a rapid distribution phase primarily to the liver. Bioavailability was approximately 22% based on a comparison of area under the plasma concentration-time curve from zero to the last measurable concentration (AUCₗₐₛₜ) after a subcutaneous versus an intravenous dose at 3 mg/kg. Plasma exposure compared to the 3 mg/kg dose group increased roughly in a dose-proportional manner for the 10 mg/kg group and a greater-than-dose proportional manner for the 100 mg/kg group. Liver and kidney exposure, based on maximum concentration observed after administration (Cₘₐₓ) and AUCₗₐₛₜ, increased approximately in a dose-proportional manner at 10 mg/kg and in a less-than-dose proportional manner at 100 mg/kg compared to the 3 mg/kg dose group. The elimination half-life in the liver ranged from 3.94 to 4.98 days

### Example 7: Effect of Compound J on CFB Expression in a CAIA-Induced Arthritis Mouse Model

The effect of Compound J on treating symptoms related to arthritis was studied using a collagen antibody-induced arthritis (CAIA) induced arthritis mouse model, which is a simple model for rheumatoid arthritis. The CAIA-induced arthritis mouse model was generated by administering a collagen antibody to the mouse on day 0, followed by administration of an LPS booster on day 3.

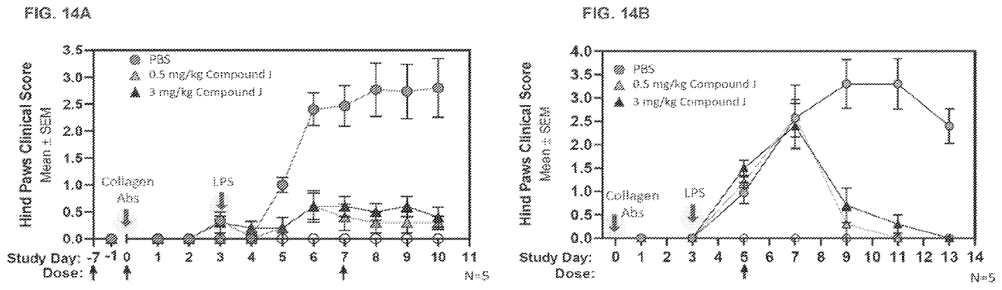

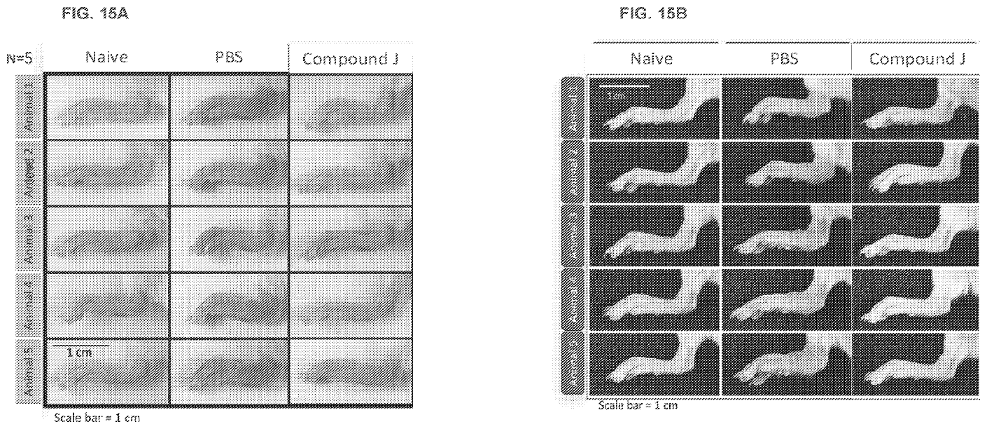

Compound J was tested in both preventative and therapeutic studies. Animals were dosed with 1.5 or 3 mg/kg of Compound J on day −7 for the preventative study (FIG. 14A) and after disease onset on day 5 for the therapeutic study (FIG. 14B). The hind paw inflammation was analyzed visually on day 10 and results from both the preventative and therapeutic studies are shown in FIGS. 15A and 15B, respectively. Prophylactic treatment with Compound J prevented the swelling of hind paws, a characteristic hallmark of this model (FIG. 15A). Therapeutic treatment with Compound J completely reverted clinical disease manifestation after a single dose when compared to PBS-treated control animals (FIG. 15B).

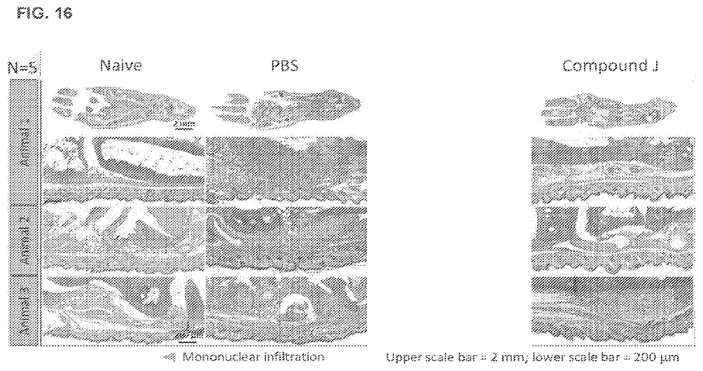

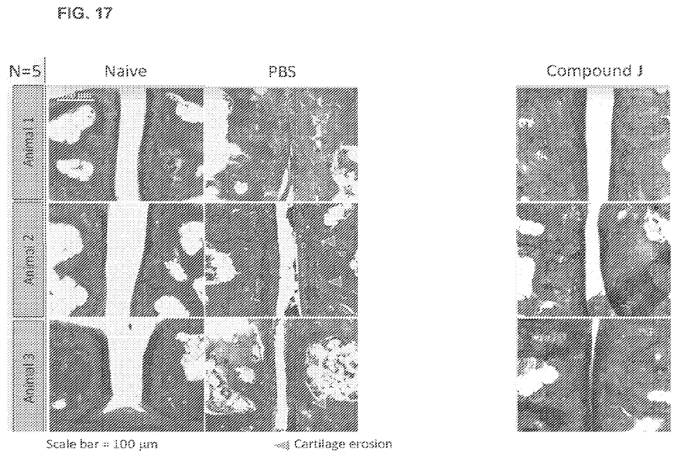

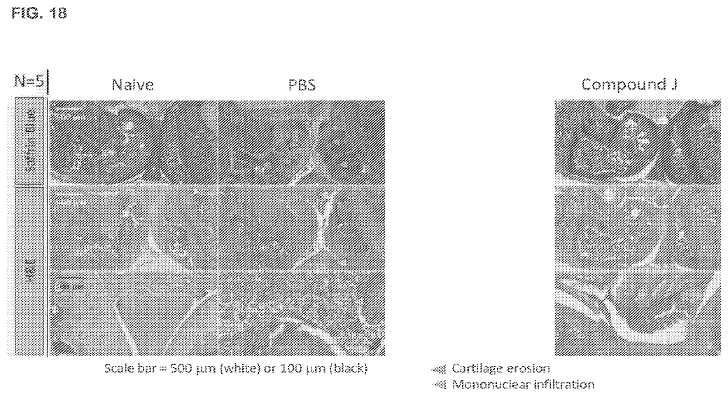

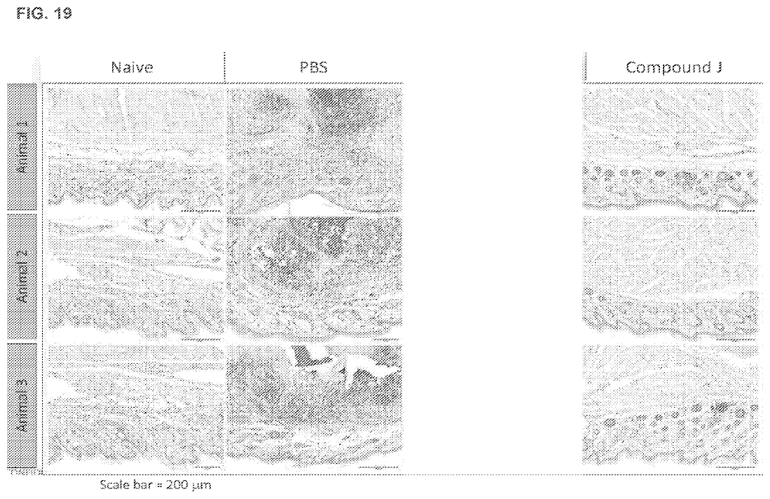

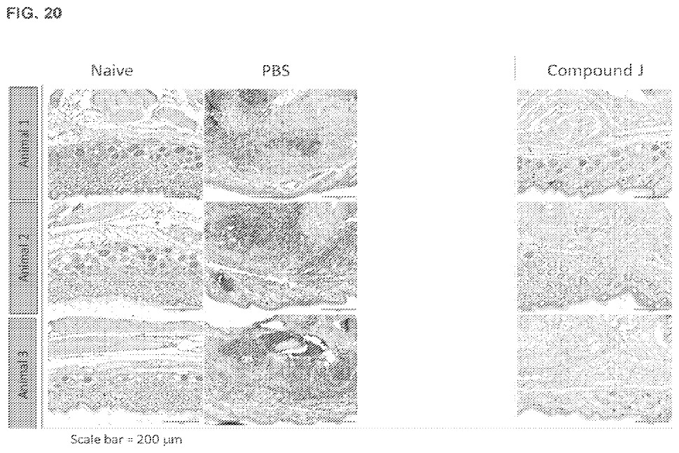

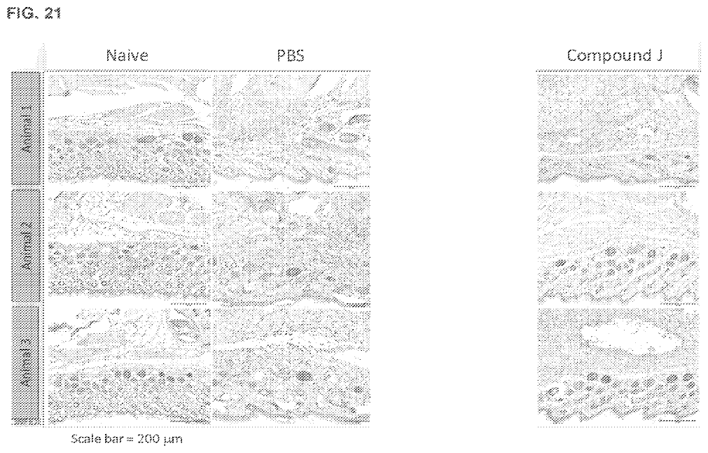

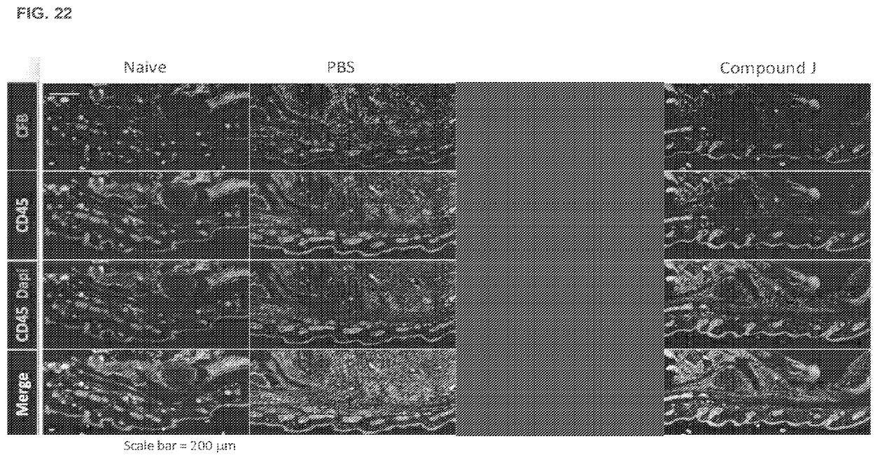

Hematoxylin and eosin (H&E) staining was performed on the biopsy of the hind paws and knees and shows a reduction of local mononuclear cells infiltration in mice that were treated preventatively with 3 doses of 3 mg/kg of Compound J (FIGS. 16 and 18). Additionally, lymphocytes (CD45 positive cells), leukocytes (CD11b positive cells) and macrophages (F4/80 positive cells) marker staining was performed on biopsy samples as shown in FIGS. 19, 20, and 21, respectively, to show the reduction of local inflammation as a result of therapeutic treatment with single 3 mg/kg dose of Compound J. Biopsy samples were also stained with Safranin O to visualize cartilage in the knees of the CAIA-induced arthritis mouse model. Animals treated with 3 mg/kg of Compound J showed a remarkable reduction in cartilage erosion in comparison to PBS-treated mice when treated preventatively (FIG. 17 and FIG. 18). Experiments using in situ hybridization to CFB and CD45 mRNAs were performed on biopsy samples in order to assess complement expression at local sites of inflammation for CAIA-induced arthritic mice with and without treatment with 3 mg/kg Compound J, which is shown in FIG. 22. The hepatic knockdown of CFB with Compound J reduced the infiltration of lymphocytes (CD45 positive cells) and the local CFB mRNA expression with therapeutic treatment with Compound J in comparison to PBS-treated animals as a control group.

### Example 8: Effect of Compound J on CFB Expression in a Multiple Sclerosis Mouse Model

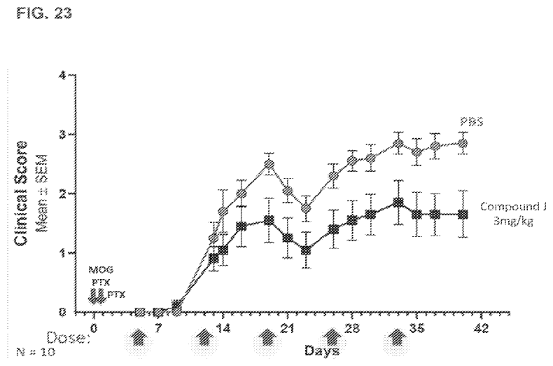

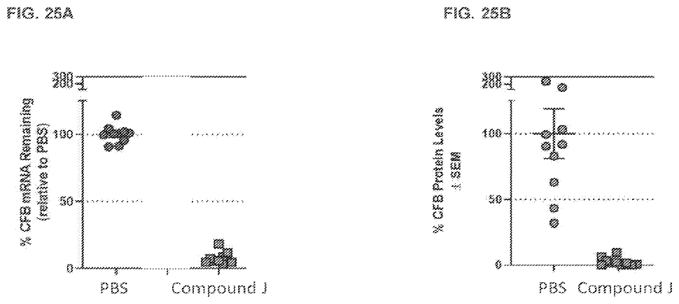

Myelin oligodendrocyte glycoprotein (MOG)-induced experimental autoimmune encephalomyelitis (EAE) mouse model can be used to investigate the immune-mediated mechanism of neuroinflammation and demyelination. MOG-induced EAE mice were treated preventatively with a 3 mg/kg dose of Compound J (n=2 experiments). The liver CFB mRNA levels after treatment with Compound J as well as the CFB protein in serum was assessed using RT-qPCR and an immunoblot respectively as shown in FIGS. 25A and 25B. Likewise, the percentage of CFB mRNA remaining after treatment with Compound J as well as the amount of CFB in serum of MOG-induced EAE mice after being treated with a dosage of 3 mg/kg of Compound J in comparison to animals treated with PBS was assessed. The hepatic knockdown of CFB with Compound J reduced the severity of the disease (FIG. 23).

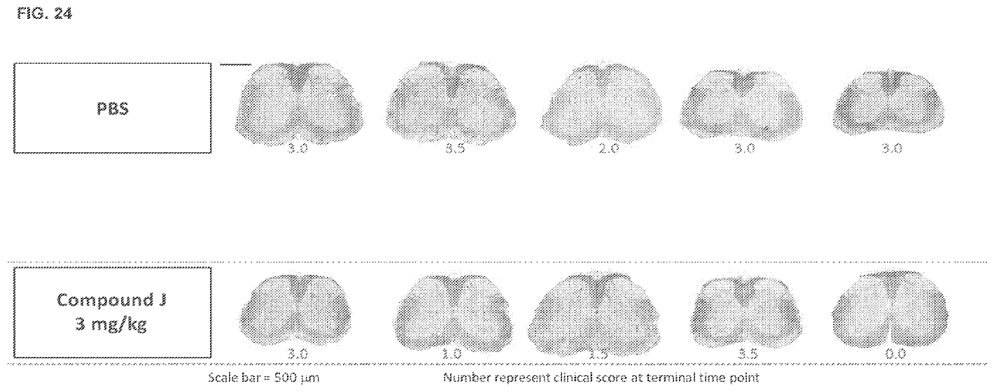

Lumbar spinal cord samples were also obtained from MOG-induced EAE mice treated with Compound J. Luxol fast blue staining along with H&E staining was performed on spinal cord samples in order to visualize myelination as well as mononuclear cell infiltration as shown in FIG. 24. Luxol fast blue spinal cord samples were compared between disease animals treated with 3 mg/kg of Compound J, PBS, as shown in FIG. 24. MOG-induced animals treated with Compound J showed a modest reduction of the de-myelination and prevention of immune cell infiltration.

### Example 9: Treating Multiple Sclerosis with Compound B in Humans

A subject suffering from multiple sclerosis can be treated with a pharmaceutical composition containing Compound B (e.g., in a dose amount of about 3 mg/kg). The subject can be administered the composition at a frequency of once a week, for example, by intramuscular injection, for a period of about 12 months or longer (e.g., until symptoms resolve or stabilize). Approximately once a month, the subject's symptoms and serum CFB levels can be evaluated by a clinician to assess the efficacy of Compound B. The subject's serum CFB can be quantified using a blood serum sample and can be compared to the amount of CFB protein found in the serum of the subject prior to being administered Compound B or relative to a control amount of CFB protein or the amount of CFB protein present in a serum sample from a normal subject (e.g., a disease-free subject). Treatment with Compound B may be determined to be effective if the amount of CFB protein in serum decreases by at least 10% in comparison to the amount of CFB protein in serum prior to treatment with Compound B. Additionally, the subject's symptoms associated with multiple sclerosis, such as blurry vision, slurred speech, dizziness, tingling, lack of coordination, and unsteady gait, can be assessed by a clinician to evaluate if there is a decrease in any or all of the symptoms a subject is experiencing in comparison to the symptoms the subject was experiencing prior to being administered Compound B.

### Example 10: Treating Arthritis with Compound B in Humans

A subject diagnosed with arthritis can be treated with a pharmaceutical composition containing Compound B (e.g., in a dose of about 1.5 mg/kg). The subject can be administered the composition at a frequency of about once a month, for example, by intramuscular injection, for a period of about 6 months or longer (e.g., until symptoms resolve or stabilize). The subject can be evaluated (e.g., by assessing the subject's symptoms and/or serum CFB levels) by a clinician to assess the efficacy of Compound B, for example, every one or two months. The subject's serum CFB can be quantified using a blood serum sample and can be compared to the amount of CFB protein found in the serum of the subject prior to being administered Compound B or relative to a control amount of CFB protein or the amount of CFB protein present in a serum sample from a normal subject (e.g., a disease-free subject). Treatment with Compound B may be determined to be effective if the amount of CFB protein in serum decreases by at least 10% in comparison to the amount of CFB protein in serum prior to treatment with Compound B. Additionally, the subject's symptoms associated with arthritis, including pain, stiffness, swelling, redness, and a decreased range of motion, can be assessed by a clinician to evaluate if there is a decrease in any or all of the symptoms a subject is experiencing in comparison to the symptoms the subject was experiencing prior to being administered Compound B.

### Example 11: Evaluation of Toxicology in Cynomolgus Monkeys and Mice

The safety and tolerability of subcutaneously administered Compound B has been evaluated in cynomolgus monkeys and mice.

In a safety pharmacology study, subcutaneous administration of 0.9% sodium chloride was followed by increasing doses of Compound B (at 30, 100 and 300 mg/kg) given once every 7 days to the same cynomolgus monkeys. No cardiovascular or neurologic effects were observed at any dose level. At 300 mg/kg, minimal lower minute volume and minimal lower tidal volume were noted during respiratory evaluations. These respiratory findings were considered non adverse based on the minimal severity and all the animals remained in good health throughout the study. Therefore, the no adverse effect level (NOAEL) in this study was determined to be 300 mg/kg.

Genetic toxicology assessments, which included in vitro micronucleus assays and an in vitro bacterial reverse mutation assays, were negative for inducing micronuclei and for mutagenic activity, respectively.

In a 6-month mouse study, the potential toxicity of repeat subcutaneous dosing (0, 30, 100, 300 mg/kg every 4 weeks for a total of 7 doses) of Compound B, and the reversibility, persistence, or delayed occurrence of any effects after an 8-week recovery period were evaluated. The range of dose levels in this study was selected to achieve at least a 10-fold exposure multiple over the expected exposure at the highest intended clinical dose. The toxicokinetic characteristics of Compound B were also determined. Nonadverse clinical pathology findings included increased neutrophil (2.08× and 1.39×, respectively), monocyte (2.90× and 2.38×, respectively), and lymphocyte counts (1.92× and 1.35×, respectively) resulting in increased total white blood cell counts (1.94× and 1.38×, respectively) in the 300 mg/kg/day group males and females at Day 171 and decreased cholesterol concentration (62.9×) in the 300 mg/kg/day group males at Day 171. Complete reversibility of all clinical pathology findings was evident at the end of the recovery period. Nonadverse microscopic findings included hepatocellular karyocytomegaly, with correlating higher liver/gallbladder weights, mixed cell inflammation of the liver, intracytoplasmic basophilic granules in the kidney, and subcutaneous infiltrates of histiocytes, mixed cell inflammation of the dermis, and degeneration, necrosis, and/or regeneration of the muscularis carnosus at the injection site at the terminal euthanasia with hepatocellular karyocytomegaly, mixed cell inflammation of the liver, subcutaneous infiltrates of the histiocytes, and degeneration, necrosis, and/or regeneration of the muscularis carnosus at the injection site still present at the recovery euthanasia.

Liver findings in these animals included microvesicular fatty change, single cell necrosis, and karyocytomegaly. Since the liver was considered a target tissue at the terminal euthanasia, the liver changes seen in these decedent mice were considered potentially Compound B related. In addition to the two main study deaths, there were 2 animals that had early mortality of unknown relation to Compound B in the toxicokinetics group. These animals were either found dead (300 mg/kg group female on Day 14) or were euthanized due to moribundity (30 mg/kg group male on Day 99). A potentially pertinent macroscopic finding from these animals was a pale discolored liver in the 30 mg/kg group male. Based on the mortality observed in the 100 and 300 mg/kg group females of the main study, the NOAEL was considered to be 30 mg/kg for females and 300 mg/kg for males. These doses corresponded to plasma mean AUClast values of 17,400 and 609,000 hr*ng/ml and mean Cmax values of 6490 and 140,000 ng/mL for the 30 mg/kg/day group females and 300 mg/kg group males, respectively, on Day 169.

In the 9-month monkey study, the potential toxicity of repeat subcutaneous dosing (0, 30, 100, 300 mg/kg every 4 weeks for a total of 10 doses of Compound B), and the reversibility, persistence, or delayed occurrence of any effects after an 8-week recovery period were evaluated. The range of dose levels in this study was selected to achieve at least a 10-fold exposure multiple over the expected exposure at the highest intended clinical dose. The toxicokinetic characteristics and pharmacodynamic effects of Compound B were assessed. At the end of the dosing phase of this study at ≥30 mg/kg, there were minimal to moderate nonadverse microscopic findings of eosinophilic hepatocellular globules in the liver and vacuolated/granular macrophages noted in multiple tissues (digestive system, genitourinary system, lymphoid system, brain (choroid plexus), eye (choroid or ciliary body), heart, adrenal gland, thyroid gland, skin, skeletal muscle, femorotibial joint, and/or at the subcutaneous administration sites). These findings persisted at the end of the recovery phase at a lower incidence and/or severity, indicating ongoing yet incomplete recovery. Exposure at all dose levels produced expected and inter-related pharmacodynamic effects as shown by a >95% decrease in serum CFB protein concentrations, and >95% decrease in complement functional activity of the Complement Alternative Pathway by Day 28 and a >89% decrease in hepatic CFB mRNA expression following euthanasia. Based on the lack of any adverse findings following the dosing period, the NOAEL was determined to be 300 mg/kg, with associated AUCₗₐₛₜ of 1360000 hr*ng/ml and Cmax of 66200 ng/ml (males and females combined, Day 253).

### Example 12: CFB Assessment Assays

Various assays including assessment of Compound B in plasma or tissue, WIESLAB® complement functional activity assays, assessment of circulating CFB, CFB mRNA expression levels, and pharmacokinetic assays may be used as described herein to characterize the effect of Compound B on CFB levels.

### Compound B in Plasma

Concentrations of Compound B in plasma of mouse and monkey were measured through high-performance liquid chromatography-fluorescence detection (HPLC-FD) analytical method. The analyte (Compound B) in 30 μL of plasma sample was enzymatically treated with Proteinase K, hybridized with a fluorescent probe (Peptide Nucleic Acid; 22-mer peptide nucleic acid PNA probe) with sequence complementarity to the antisense strand of Compound B, and injected into a high-performance liquid chromatography (HPLC) equipped with a fluorescence detector. Chromatographic separation was performed using a gradient system on Shimadzu Prominence systems using DNAPAC™ PA200 analytical columns. The mobile phases were 30% Acetonitrile (25 mM Tris HCl, 1 mM EDTA, 2M Urea) for mobile phase A and 1M NaClO₄ in mobile phase A for mobile phase B. FL Detector monitored signals from 436 nm (Ex) to 484 nm (Em). Compound B concentrations were calculated using LabSolutions 6.70 with a linear regression using the least squares method (with 1/c2 weighting) over a quantification range of 2.00 ng/ml to 2000 ng/ml with the low and high ends of these ranges defining the lower limit of Quantification (LLOQ) and upper limit of quantification (ULOQ), respectively. This assay was used, for example, in Examples 5 and 6 as described above.

### Compound B in Tissue

Concentrations of Compound B in liver and kidney in both monkey and mouse were measured through HPLC-FD analytical method. Using a 2.5 mg tissue sample in 50-μL aliquot volume (tissue homogenate), Compound B in tissue samples was enzymatically treated with Proteinase K and followed by hybridization with the 22-mer PNA probe that had sequence complementarity to the antisense strand of Compound B. Processed samples were injected into an HPLC equipped with a fluorescence detector. Chromatographic separation was performed using a gradient system on a Shimadzu Prominence system using a DNAPAC™ PA200 analytical columns. The mobile phases are 30% Acetonitrile (25 mM Tris HCl, 1 mM EDTA, 2M Urea) for mobile phase A and 1M NaClO₄ in mobile phase A for mobile phase B. Compound B concentrations were calculated using LabSolutions 6.89 with a linear regression using the least squares method (with 1/c2 weighting) over a quantification range of 30.0 ng/g to 20,000 ng/g with the low and high ends of these ranges defining the LLOQ and ULOQ, respectively. This assay was used, for example, in Examples 5 and 6 as described above.

### Wieslab® Complement Functional Activity Assay (CCP, CAP, CLP)

The complement classical pathway (CCP), CAP, and complement lectin pathway activities were evaluated using a WIESLAB® Complement System Screen assay, using labeled antibodies specific for a neoantigen to detect the human terminal complement complex (C5b-9) complex produced as a result of complement activation. The assay is also able to detect cynomolgus monkey C5b-9. The amount of neoantigen generated was proportional to the level of functional activity of the individual pathways. Wells in the assay's microtiter strips were coated with specific activators of the classical, or the alternative, or the lectin pathways. Monkey serum samples were diluted in diluent containing a blocker which ensures that only the respective pathway was activated. The wells were washed, and C5b-9 was detected with a specific alkaline phosphatase-labeled antibody to the neoantigen expressed. The amount of complement activation correlated with the color intensity measured by absorbance at 405 nm. The value for the positive control provided in the test kit was defined as 100% complement activation. All measured values were expressed as percent (%) complement activity, determined as follows:

[(Sample−negative control)/(positive control−negative control)]*100

This assay was used, for example, in Example 5 above.

### Protein Level

The amount of cynomolgus circulating Factor B protein levels was evaluated by Western Blot using measurement of relative CFB serum protein concentrations normalized to the level of Transferrin (TF) in monkey samples. Diluted serum samples mixed in sample buffer were combined with fluorescent master mix. Samples were boiled at 95° C. for 5 minutes, vortexed and spun down. Samples were then placed on ice and run in Western Blot. Quantification was performed using ProteinSimple software according to the manufacturer's instructions. The degree of CFB protein reduction in the treatment groups was calculated as the percent of expression relative to the average level of the PBS-treated control group on the same study day where monkey CFB levels in the PBS-treated control group was set at 100%. Graphs of mean±standard deviation were generated in and data were analyzed using GraphPad Prism (GraphPad Software, La Jolla, CA). An unpaired t test was performed to compare monkey CFB protein levels in Compound B-treated groups relative to the PBS-treated control group from the same study day.

Assessment of circulating Factor B protein levels was also evaluated using a Factor B enzyme-linked immunosorbent assay (ELISA) kit designed for the quantitative measurement of Complement Factor B concentrations in Human. A complement Factor B specific antibody was precoated onto 96-well plates and blocked. Standards or test samples were added to the wells and subsequently a Complement Factor B specific biotinylated detection antibody was added and then followed by washing with wash buffer. Streptavidin-Peroxidase Conjugate was added and unbound conjugates were washed away with wash buffer. Tetramethylbenzidine (TMB) was then used to visualize Streptavidin-Peroxidase enzymatic reaction. TMB was catalyzed by Streptavidin-Peroxidase to produce a blue color product that changes into yellow after adding acidic stop solution. The density of yellow coloration was directly proportional to the amount of Complement Factor B capture in plate. Back-calculated concentration of the sample was determined by the curve fitting regression program generated by the calibration standards. This assay was used, for example, in Example 5 as described above.

CFB mRNA Expression Levels

The amount of factor B in cynomolgus liver was determined by measuring Factor B mRNA expression relative to peptidyl-prolyl cis-trans isomerase B (PPIB) mRNA expression in RNA isolated from cynomolgus monkey liver using a duplex real-time quantitative polymerase chain reaction (qPCR) assay following reverse transcription. mRNA was first isolated from frozen liver tissue, and quantified. Subsequently, mRNA was transcribed into complementary DNA (cDNA). The cDNA was then used as the template for the qPCR reaction to measure Factor B mRNA level with normalization to PPIB. The degree of Factor B mRNA in the treated groups was calculated as the percent of expression (normalized to PPIB mRNA levels) relative to untreated or the pre-dose group, where Factor B mRNA expression in the control group was set at 100%. This assay was used, for example, in Examples 5 and 11 as described above.

### Pharmacokinetic Assays

Concentrations of Compound B in human plasma are measured through HPLC-FD analytical method. The analyte (Compound B) in 30 μL of plasma sample is enzymatically treated with Proteinase K, hybridized with a fluorescent probe (Peptide Nucleic Acid; 22-mer PNA probe) having sequence complementarity to the antisense strand of Compound B, and injected into an HPLC equipped with a fluorescence detector. Chromatographic separation is performed using a gradient system on Shimadzu Prominence systems using DNAPAC™ PA200 analytical columns. The mobile phases are 30% Acetonitrile (25 mM Tris HCl, 1 mM EDTA, 2M Urea) for mobile phase A and 1M NaClO₄ in mobile phase A for mobile phase B. FL Detector monitored signals from 436 nm (Ex) to 484 nm (Em). Compound B concentrations is calculated using LabSolutions 6.70 with a linear regression using the least squares method (with 1/c2 weighting) over a quantification range of 2.00 ng/ml to 2000 ng/ml with the low and high ends of these ranges defining the LLOQ and ULOQ, respectively.

### Antidrug Antibody Assay

An antidrug antibody (ADA) assay for Compound B can be performed using human serum and an electrochemiluminescence (ECL) bridging assay. Positive controls (PCs) are generated from rabbits immunized against an immunogenic cocktail consisting of keyhole limpet hemocyanin (KLH)-conjugated Compound B and KLH-conjugated oligonucleotides of various lengths corresponding to modified Compound B sequences. The PCs, negative controls (NCs), and study samples can be subjected to an acid dissociation step at ambient room temperature then added to a plate containing TRIS, biotin-Compound B, and ruthenium-labeled Compound B, enabling formation of bridging complexes between the labeled Compound B and the Compound B antibodies present in the sample. After incubation, NC, PC, and study samples can be transferred to a streptavidin-coated plate and incubated in the dark for 1 hour during which drug binds to the plate capturing the ADA bridging complex. The plate is then washed, and an MESO SCALE DISCOVERY® (MSD®) read buffer is added to generate an ECL signal which is directly proportional to the amount of ADA present in the sample.

### Example 13: Effect of Compound J in Membranous Nephropathy Rat Model

The effect of Compound J on symptoms related to membranous nephropathy was studied using the Passive Heyman's Nephritis (PHN) rat model, which is a simple model for membranous nephropathy. The PHN rat model was generated by administering a single dose of sheep anti-rat FX1a antibody to the rat on day 0.

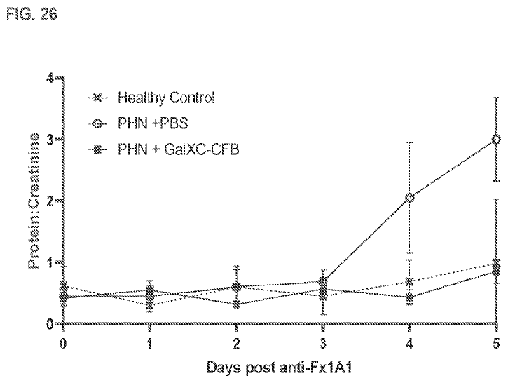

Compound J was tested in a preventative study. In this study, animals were dosed with 12 mg/kg of Compound J on day −14, −7, and 0 (FIG. 26). Kidney function was assessed as the ratio of protein:creatinine levels measured from urine samples collected daily from animals as shown in FIG. 26. Prophylactic treatment with Compound J prevented the development of proteinuria when compared to PBS-treated PHN control animals, a characteristic hallmark of this model (FIG. 26).

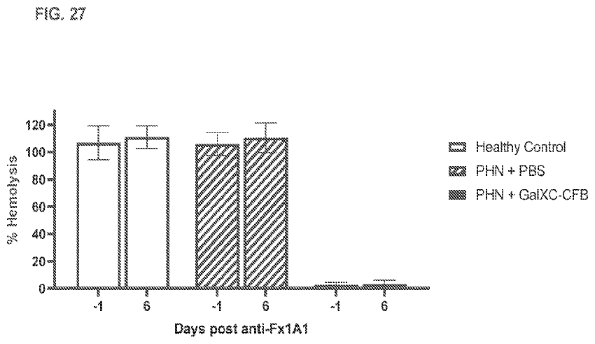

Serum samples were collected on pre-disease induction Day −1 and 6-days post-disease induction for evaluation of complement activity by hemolysis of rabbit erythrocytes (FIG. 27). Healthy and PBS-treated PHN animals were used as control for CFB functional assays. PHN rats after receiving 2 (day −1 serum) or 3 doses (Day 6) with Compound J showed a >95% reduction of alternative pathway complement activity as measured by the lysis of rabbit erythrocytes in a hemolytic assay when compared to the lysis level observed from either healthy or PBS-treated PHN control animals (FIG. 27). PBS was administered in the same multidose regimen to comprise a disease control group, and sham animals were used as healthy controls.

### OTHER EMBODIMENTS

All publications, patents, and patent applications mentioned in this specification are herein incorporated by reference to the same extent as if each independent publication or patent application was specifically and individually indicated to be incorporated by reference. While some embodiments are herein described one of skill in the art will appreciate that further modifications and embodiments are encompassed including variations, uses or adaptations generally following the principles described herein and including such departures from the present disclosure that come within known or customary practice within the art and may be applied to the essential features hereinbefore set forth, and follows in the scope of the claims.

1. An RNAi oligonucleotide, or a pharmaceutically acceptable salt thereof, for reducing complement factor B (CFB) expression, the oligonucleotide comprising a sense strand and an antisense strand, wherein the sense strand and the antisense strand form a duplex region, wherein the antisense strand comprises a region of complementarity to a CFB mRNA target sequence of SEQ ID NO: 13 or 14, and wherein the region of complementarity is at least 20 contiguous nucleotides in length, and wherein the sense strand comprises a nucleotide sequence as set forth in SEQ ID NO: 37 and the antisense strand comprises a nucleotide sequence as set forth in SEQ ID NO: 38.

2.-7. (canceled)

8. The RNAi oligonucleotide of claim 1, or a pharmaceutically acceptable salt thereof, wherein the 3′ end of the sense strand comprises a stem-loop set forth as S1-L-S2, wherein S1 is complementary to S2, and wherein L forms a loop between S1 and S2 of 3-5 nucleotides in length.

9. (canceled)

10. The RNAi oligonucleotide of claim 8, or a pharmaceutically acceptable salt thereof, wherein L is a tetraloop.

11. The RNAi oligonucleotide of claim 10, or a pharmaceutically acceptable salt thereof, wherein the tetraloop comprises the nucleic acid sequence of GAAA.

12.-65. (canceled)

66. A pharmaceutical composition comprising the RNAi oligonucleotide of claim 1, or a pharmaceutically acceptable salt thereof, and a pharmaceutically acceptable carrier, excipient, or diluent.

67. A method for treating a subject having a disease, disorder, or condition mediated by complement pathway activation or dysregulation, the method comprising administering to the subject the RNAi oligonucleotide of claim 1, or a pharmaceutically acceptable salt thereof.

68.-74. (canceled)

75. The method of claim 67, wherein the subject is a human.

76. (canceled)

77. The method of claim 67, wherein the disease, disorder, or condition mediated by complement pathway activation or dysregulation is selected from the group consisting of paroxysmal nocturnal hemoglobinuria (PNH), C3 glomerulopathy (C3G), immunoglobulin A nephropathy (IgAN), membranous nephropathy (MN), including primary MN, E. coli-induced or typical hemolytic uremic syndrome (HUS), atypical hemolytic uremic syndrome (aHUS), age-related macular degeneration, geographic atrophy, diabetic retinopathy, uveitis, intermediate uveitis, Behcet's uveitis, retinitis pigmentosa, macular edema, multifocal choroiditis, Vogt-Koyanagi-Harada syndrome, birdshot retinochoriodopathy, sympathetic ophthalmia, ocular cicatricial pemphigoid, ocular pemphigus, nonarthritic ischemic optic neuropathy, post-operative inflammation, retinal vein occlusion, neurological disorders, multiple sclerosis, stroke, Guillain Barre Syndrome, traumatic brain injury, Parkinson's disease, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, arthritis, autoimmune heart disease, inflammatory bowel disease, ischemia-reperfusion injuries, Barraquer-Simons Syndrome, hemodialysis, anti-neutrophil cytoplasmic antibody (ANCA) vasculitis, cryoglobulinemia, psoriasis, transplantation, diseases of the central nervous system such as Alzheimer's disease and other neurodegenerative conditions, dense deposit disease, blistering cutaneous diseases, membranoproliferative glomerulonephritis type II (MPGN II), chronic graft vs. host disease, Felty syndrome, pyoderma gangrenosum (PG), hidradenitis suppurativa (HS), pulmonary arterial hypertension, primary Sjogren's syndrome, primary biliary cholangitis, autosomal dominant polycystic kidney disease, and myelin oligodendrocyte glycoprotein antibody disease (MOGAD).

78. The method of claim 67, wherein the disease, disorder, or condition mediated by complement pathway activation or dysregulation is rheumatoid arthritis.

79.-81. (canceled)

82. The method of claim 67, wherein the RNAi oligonucleotide, or a pharmaceutically acceptable salt thereof, is administered at a dose of between about 0.1 mg/kg to about 150 mg/kg.

83. A method for reducing CFB expression in a cell, a population of cells, or a subject, the method comprising the step of:
i) contacting the cell or the population of cells with an RNAi oligonucleotide comprising a sense strand and an antisense strand, wherein the sense strand and the antisense strand form a duplex region, wherein the antisense strand comprises a region of complementarity to a CFB mRNA target sequence of SEQ ID NO: 13 or 14, and wherein the region of complementarity is at least 20 contiguous nucleotides in length, and wherein the sense strand comprises a nucleotide sequence as set forth in SEQ ID NO: 37 and the antisense strand comprises a nucleotide sequence as set forth in SEQ ID NO: 38, or a pharmaceutically acceptable salt thereof; or
ii) administering to the subject an RNAi oligonucleotide comprising a sense strand and an antisense strand, wherein the sense strand and the antisense strand form a duplex region, wherein the antisense strand comprises a region of complementarity to a CFB mRNA target sequence of SEQ ID NO: 13 or 14, and wherein the region of complementarity is at least 20 contiguous nucleotides in length, and wherein the sense strand comprises a nucleotide sequence as set forth in SEQ ID NO: 37 and the antisense strand comprises a nucleotide sequence as set forth in SEQ ID NO: 38, or a pharmaceutically acceptable salt thereof.

84.-89. (canceled)

90. A kit comprising the RNAi oligonucleotide of claim 1, or a pharmaceutically acceptable salt thereof.

91.-94. (canceled)

95. The RNAi oligonucleotide of claim 1, wherein the RNAi oligonucleotide comprises a pharmaceutically acceptable salt.

96. The RNAi oligonucleotide of claim 95, wherein the pharmaceutically acceptable salt is or comprises acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate, methylamine, dimethylamine, trimethylamine, triethylamine, or ethylamine, or is an alkali or alkaline earth metal salt.

97. The RNAi oligonucleotide of claim 96, wherein the alkali or alkaline earth metal salt is selected from the group consisting of sodium, lithium, potassium, calcium, and magnesium.

98. The RNAi oligonucleotide of claim 95, wherein the pharmaceutically acceptable salt is a sodium salt.

99. A method for treating a subject having a disease, disorder, or condition mediated by complement pathway activation or dysregulation, the method comprising administering to the subject the pharmaceutical composition of claim 66.

100. The method of claim 99, wherein the pharmaceutical composition is formulated for daily, weekly, monthly, or yearly administration.

101. The method of claim 99, wherein the pharmaceutical composition is formulated for intravenous, subcutaneous, intramuscular, oral, nasal, sublingual, intrathecal, or intradermal administration.

102. The method of claim 101, wherein the pharmaceutical composition is formulated for subcutaneous administration.


SEQUENCE LISTING (78 sequences)

SEQ ID NO: 1 - RNA, 36 nt, synthetic construct
  source 1..36: mol_type = other RNA
caggaattcctgaattttaagcagccgaaaggctgc

SEQ ID NO: 2 - RNA, 20 nt, synthetic construct
  source 1..20: mol_type = other RNA
caggaattcctgaattttaa

SEQ ID NO: 3 - RNA, 22 nt, synthetic construct
  source 1..22: mol_type = other RNA
ttaaaattcaggaattcctggg

SEQ ID NO: 4 - RNA, 36 nt, synthetic construct
  source 1..36: mol_type = other RNA
acaatgtgagtgatgagatagcagccgaaaggctgc

SEQ ID NO: 5 - RNA, 20 nt, synthetic construct
  source 1..20: mol_type = other RNA
acaatgtgagtgatgagata

SEQ ID NO: 6 - RNA, 22 nt, synthetic construct
  source 1..22: mol_type = other RNA
tatctcatcactcacattgtgg

SEQ ID NO: 7 - RNA, 16 nt, synthetic construct
  source 1..16: mol_type = other RNA
gcagccgaaaggctgc

SEQ ID NO: 8 - skipped

SEQ ID NO: 9 - RNA, 58 nt, synthetic construct
  source 1..58: mol_type = other RNA
caggaattcctgaattttaagcagccgaaaggctgcttaaaattcaggaattcctggg

SEQ ID NO: 10 - RNA, 58 nt, synthetic construct
  source 1..58: mol_type = other RNA
acaatgtgagtgatgagatagcagccgaaaggctgctatctcatcactcacattgtgg

SEQ ID NO: 11 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 12 - RNA, 2646 nt, Homo sapiens
  source 1..2646: mol_type = mRNA
gacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacaaaaaaaaaaaaaaaaaaaa

SEQ ID NO: 13 - DNA, 19 bp, synthetic construct
  source 1..19: mol_type = other DNA
caggaattcctgaatttta

SEQ ID NO: 14 - DNA, 19 bp, synthetic construct
  source 1..19: mol_type = other DNA
acaatgtgagtgatgagat

SEQ ID NO: 15 - RNA, 36 nt, synthetic construct
  source 1..36: mol_type = other RNA
gtgaccagatttcttttcaagcagccgaaaggctgc

SEQ ID NO: 16 - RNA, 23 nt, synthetic construct
  source 1..23: mol_type = other RNA
tttgaaaagaaatctggtcacgg

SEQ ID NO: 17 - RNA, 36 nt, synthetic construct
  source 1..36: mol_type = other RNA
gtgatgagatctctttccaagcagccgaaaggctgc

SEQ ID NO: 18 - RNA, 22 nt, synthetic construct
  source 1..22: mol_type = other RNA
ttggaaagagatctcatcacgg

SEQ ID NO: 19 - RNA, 36 nt, synthetic construct
  source 1..36: mol_type = other RNA
tctacctggtgctagatggagcagccgaaaggctgc

SEQ ID NO: 20 - RNA, 22 nt, synthetic construct
  source 1..22: mol_type = other RNA
tccatctagcaccaggtagagg

SEQ ID NO: 21 - RNA, 36 nt, synthetic construct
  source 1..36: mol_type = other RNA
aaaagtgtctagtcaacttagcagccgaaaggctgc

SEQ ID NO: 22 - RNA, 22 nt, synthetic construct
  source 1..22: mol_type = other RNA
taagttgactagacacttttgg

SEQ ID NO: 23 - RNA, 36 nt, synthetic construct
  source 1..36: mol_type = other RNA
ccaattactgtcattgatgagcagccgaaaggctgc

SEQ ID NO: 24 - RNA, 22 nt, synthetic construct
  source 1..22: mol_type = other RNA
tcatcaatgacagtaattgggg

SEQ ID NO: 25 - RNA, 36 nt, synthetic construct
  source 1..36: mol_type = other RNA
aggattatctggatgtctaagcagccgaaaggctgc

SEQ ID NO: 26 - RNA, 22 nt, synthetic construct
  source 1..22: mol_type = other RNA
ttagacatccagataatcctgg

SEQ ID NO: 27 - RNA, 36 nt, synthetic construct
  source 1..36: mol_type = other RNA
acaagagaagtcgtttcatagcagccgaaaggctgc

SEQ ID NO: 28 - RNA, 22 nt, synthetic construct
  source 1..22: mol_type = other RNA
tatgaaacgacttctcttgtgg

SEQ ID NO: 29 - RNA, 36 nt, synthetic construct
  source 1..36: mol_type = other RNA
aagtcgtttcattcaagttagcagccgaaaggctgc

SEQ ID NO: 30 - RNA, 22 nt, synthetic construct
  source 1..22: mol_type = other RNA
taacttgaatgaaacgacttgg

SEQ ID NO: 31 - DNA, 20 bp, synthetic construct
  source 1..20: mol_type = other DNA
gtgaccagatttcttttcaa

SEQ ID NO: 32 - RNA, 2767 nt, Mus musculus
  source 1..2767: mol_type = mRNA
gctccatcacacagtccatggaaagactgatcttttaaattgggggtagtggaggtggtggtctgtgcttgttaggaggggtctgggggctaagagggagctttgaaagggaagttctggcccttggtcagtcaagggtggggctcacatagtttctgtttcctcagttggcagttcagctggggccctccttcatgaatgttccgggaagcagtggctgcgtgcgcagggtaggctggccaggctgcagatgccagagcagattgcataaaaggttaggggacagtgggaaaggggtgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacagattaaagcagttacaataacaaaaaaaaaaaaaaaaaaaaaa

SEQ ID NO: 33 - DNA, 36 bp, synthetic construct
  source 1..36: mol_type = other DNA
caggaattcctgaattttaagcagccgaaaggctgc

SEQ ID NO: 34 - DNA, 22 bp, synthetic construct
  source 1..22: mol_type = other DNA
ttaaaattcaggaattcctggg

SEQ ID NO: 35 - DNA, 36 bp, synthetic construct
  source 1..36: mol_type = other DNA
acaatgtgagtgatgagatagcagccgaaaggctgc

SEQ ID NO: 36 - DNA, 22 bp, synthetic construct
  source 1..22: mol_type = other DNA
tatctcatcactcacattgtgg

SEQ ID NO: 37 - DNA, 36 bp, synthetic construct; identical to SEQ ID NO: 35
  source 1..36: mol_type = other DNA
  modified_base 1: mod_base = OTHER; note = 2'-O-methyladenosine phosphorothioate
  modified_base 2: mod_base = OTHER; note = 2'-O-methylcytidine
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-O-methyluridine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorouridine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methyladenosine
  modified_base 17: mod_base = OTHER; note = 2'-O-methylguanosine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA

SEQ ID NO: 38 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluoroadenosine phosphorothioate
  modified_base 3: mod_base = OTHER; note = 2'-fluorouridine
  modified_base 4: mod_base = OTHER; note = 2'-fluorocytidine
  modified_base 5: mod_base = OTHER; note = 2'-fluorouridine
  modified_base 6: mod_base = OTHER; note = 2'-O-methylcytidine
  modified_base 7: mod_base = OTHER; note = 2'-fluoroadenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-fluoroadenosine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylcytidine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methylguanosine
  modified_base 20: mod_base = OTHER; note = 2'-O-methyluridine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
tatctcatcactcacattgtgg

SEQ ID NO: 39 - DNA, 21 bp, synthetic construct
  source 1..21: mol_type = other DNA
gactcggaaggaggtctacat

SEQ ID NO: 40 - DNA, 23 bp, synthetic construct
  source 1..23: mol_type = other DNA
cctctgagatgtccttgactttg

SEQ ID NO: 41 - DNA, 20 bp, synthetic construct
  source 1..20: mol_type = other DNA
aacagaagcggaagatcgtc

SEQ ID NO: 42 - DNA, 20 bp, synthetic construct
  source 1..20: mol_type = other DNA
tcacaccataacttgccacc

SEQ ID NO: 43 - DNA, 19 bp, synthetic construct
  source 1..19: mol_type = other DNA
aatgaactgcaggacgagg

SEQ ID NO: 44 - DNA, 21 bp, synthetic construct
  source 1..21: mol_type = other DNA
aggtgagatgacaggagatcc

SEQ ID NO: 45 - DNA, 21 bp, synthetic construct
  source 1..21: mol_type = other DNA
ctttccttggtcaggcagtat

SEQ ID NO: 46 - DNA, 20 bp, synthetic construct
  source 1..20: mol_type = other DNA
caacacttcgtggagtcctt

SEQ ID NO: 47 - DNA, 21 bp, synthetic construct
  source 1..21: mol_type = other DNA
cggaaggaggtgtacatcaag

SEQ ID NO: 48 - DNA, 23 bp, synthetic construct
  source 1..23: mol_type = other DNA
gaggcatctttgaccttctcata

SEQ ID NO: 49 - DNA, 20 bp, synthetic construct
  source 1..20: mol_type = other DNA
caaactttgctttccctggt

SEQ ID NO: 50 - DNA, 21 bp, synthetic construct
  source 1..21: mol_type = other DNA
caacaaagtctggcctgtatc

SEQ ID NO: 51 - RNA, 2475 nt, Macaca fascicularis
  source 1..2475: mol_type = mRNA
aggggaagggaatgtgaccaggtctaggtctggagtttcagcttggacactgagctaagtagacaagcaaaacaagccaggacacgccatcctgccccaggcccagcttctctcctgccttctaatgccatggggagcagtctcagcccccagctctacctgatgcccttcatcttgggcctcttatctggaggtgtgaccaccactccattgtcttcggcccagcctcaaggatcctgctctctggagggggtagagatcaaaggtggctccttccgacttctccaagagggccaggcactggaatacgtgtgtccttctggcttctacccgtaccctgtgcagacacgtacctgcagatccacggggtcctggagcaccctgcagactcaagatcgaaaaactgtcaagaaggcagagtgcagagcaatccgctgtccacgaccacaggacttcgagaacggggaataccggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccaggcatccccattggcacaaggaaggtgggcagccggtaccgccttgaagacagcgtcacctaccactgcagccgggggcttaccctgcgtggctcccagcggcgaacatgtcaggaaggtggctcttggagcgggacggagccttcctgccaagactccttcatgtacgacacccctcaagaggtggccgaagctttcctgtcttccctgacggagaccatagaaggagtcgatgccgaggatgggcacagcccaggggaacaacagaagcggaggatcatcctagacccttcaggctccatgaacatctacctggtgctagatggatcagacagcattggggccggcaacttcacaggagccaaaaagtgtctagtcaacttaattgagaaggtggcaagttatggtgtgaagccaagatatgctctagtgacatatgccacataccccagaatttgggtcaaagtgtctgaccaagagagcagcaatgcagactgggtcacgaagaagctcagtgaaatcaattatgaagaccacaagttgaagtcagggactaacaccaagagggccctccaggcagtgtacagcatgatgagttggccagaggacatccctcctgaaggctggaaccgcacccgccatgtcatcatcctcatgaccgatggattgcacaacatgggcggggacccaattactgtcattgatgagatccgggacttgttatacatcggcaaggatcgcaaaaacccgagggaggattatctggatgtctatgtgtttggggttggacctttggtggaccaagtgaacatcaatgctttggcttccaagaaagacaatgagcaacatgtgttcaaagtcaaggatatggaaaacctggaagacgttttcttccaaatgattgatgaaagccagtctctgagtctctgtggcatggtttgggaacacacgacgggtaccgattaccacaagcaaccatggcaggccaagatctcagtcactcgcccttcgaagggacatgagagctgtatgggggctgtggtgtctgagtactttgtgctgacagcagcacattgttttactgtggacgacaaggaacactcgatcaaggtcagcgtggggaagaagcgggacctggagatagaaaaagtcctatttcaccccgactacaacattagcgggaaaaaagaagcaggaattcctgaattttatgactatgacgttgccctgatcaagctcaagaaaaagttgaattatgacccgactatcaggcccatttgtctcccctgtaccgagggaacaactcgagctttgaggcttcctccaactaccacttgccagcaacagaaggaagagctgctccctgcacaggatatcaaagctctgtttgtgtctgaggaggagaagaagctgactcggaaggaggtctacatcaagaatggggataagaaaggcagctgtgagagagatgctcaatatgccccaggctatgacaaagtcaaggacatctcggaggtggtcacccctcggttcctttgtactggaggagtgagtccctatgctgaccccaatacttgcagaggtgattctggcggccccttgatagttcacaagagaagtcgtttcattcaagttggtgtcatcagctggggagtagtggatgtctgcaaaaaccagaagcggcaaaagcaggtacctgctcacgcccgagactttcacgtcaacctcttccaagtgctgccctggctgaaggagaaactccaagatgaggatttgggttttctctaaggggtttcctgctggacaggggcgcgggattgaattaaaacagctgcgacaaca

SEQ ID NO: 52 - DNA, 36 bp, synthetic construct
  source 1..36: mol_type = other DNA
  modified_base 1: mod_base = OTHER; note = 2'-O-methylguanosine phosphorothioate
  modified_base 2: mod_base = OTHER; note = 2'-O-methyluridine
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
gtgatgagatctctttccaagcagccgaaaggctgc

SEQ ID NO: 53 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluorouridine phosphorothioate
  modified_base 3: mod_base = OTHER; note = 2'-fluoroguanosine
  modified_base 4: mod_base = OTHER; note = 2'-fluoroguanosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-fluoroguanosine
  modified_base 11: mod_base = OTHER; note = 2'-O-methyladenosine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methyladenosine
  modified_base 20: mod_base = OTHER; note = 2'-O-methylcytidine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
ttggaaagagatctcatcacgg

SEQ ID NO: 54 - DNA, 36 bp, synthetic construct
  source 1..36: mol_type = other DNA
  modified_base 1: mod_base = OTHER; note = 2'-O- methyluridine phosphorothioate
  modified_base 2: mod_base = OTHER; note = 2'-O-methylcytidine
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-O-methyluridine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methylguanosine
  modified_base 19: mod_base = OTHER; note = 2'-O-methylguanosine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
tctacctggtgctagatggagcagccgaaaggctgc

SEQ ID NO: 55 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluorocytidine phosphorothioate
  modified_base 3: mod_base = OTHER; note = 2'-fluorocytidine
  modified_base 4: mod_base = OTHER; note = 2'-fluoroadenosine
  modified_base 5: mod_base = OTHER; note = 2'-fluorouridine
  modified_base 6: mod_base = OTHER; note = 2'-O-methylcytidine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-fluorocytidine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methyluridine
  modified_base 18: mod_base = OTHER; note = 2'-O-methyladenosine
  modified_base 19: mod_base = OTHER; note = 2'-O-methylguanosine
  modified_base 20: mod_base = OTHER; note = 2'-O-methyladenosine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
tccatctagcaccaggtagagg

SEQ ID NO: 56 - DNA, 36 bp, synthetic construct
  source 1..36: mol_type = other DNA
  modified_base 1: mod_base = OTHER; note = 2'-O-methyladenosine phosphorothioate
  modified_base 2: mod_base = OTHER; note = 2'-O-methyladenosine
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methyluridine
  modified_base 7: mod_base = OTHER; note = 2'-O-methylguanosine
  modified_base 8: mod_base = OTHER; note = 2'-fluorouridine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methyluridine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
aaaagtgtctagtcaacttagcagccgaaaggctgc

SEQ ID NO: 57 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluoroadenosine phosphorothioate
  modified_base 3: mod_base = OTHER; note = 2'-fluoroadenosine
  modified_base 4: mod_base = OTHER; note = 2'-fluoroguanosine
  modified_base 5: mod_base = OTHER; note = 2'-fluorouridine
  modified_base 6: mod_base = OTHER; note = 2'-O-methyluridine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-O-methylcytidine
  modified_base 10: mod_base = OTHER; note = 2'-fluorouridine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methyluridine
  modified_base 20: mod_base = OTHER; note = 2'-O-methyluridine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
taagttgactagacacttttgg

SEQ ID NO: 58 - DNA, 36 bp, synthetic construct
  source 1..36: mol_type = other DNA
  modified_base 1: mod_base = OTHER; note = 2'-O-methylcytidine phosphorothioate
  modified_base 2: mod_base = OTHER; note = 2'-O-methylcytidine
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyluridine
  modified_base 6: mod_base = OTHER; note = 2'-O-methyluridine
  modified_base 7: mod_base = OTHER; note = 2'-O-methyladenosine
  modified_base 8: mod_base = OTHER; note = 2'-fluorocytidine
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorouridine
  modified_base 12: mod_base = OTHER; note = 2'-O-methylcytidine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylguanosine
  modified_base 17: mod_base = OTHER; note = 2'-O-methyladenosine
  modified_base 18: mod_base = OTHER; note = 2'-O-methyluridine
  modified_base 19: mod_base = OTHER; note = 2'-O-methylguanosine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
ccaattactgtcattgatgagcagccgaaaggctgc

SEQ ID NO: 59 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluorocytidine phosphorothioate
  modified_base 3: mod_base = OTHER; note = 2'-fluoroadenosine
  modified_base 4: mod_base = OTHER; note = 2'-fluorouridine
  modified_base 5: mod_base = OTHER; note = 2'-fluorocytidine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyluridine
  modified_base 9: mod_base = OTHER; note = 2'-O-methylguanosine
  modified_base 10: mod_base = OTHER; note = 2'-fluoroadenosine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methylguanosine
  modified_base 20: mod_base = OTHER; note = 2'-O-methylguanosine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
tcatcaatgacagtaattgggg

SEQ ID NO: 60 - DNA, 36 bp, synthetic construct
  source 1..36: mol_type = other DNA
  modified_base 1: mod_base = OTHER; note = 2'-O-methyladenosine phosphorothioate
  modified_base 2: mod_base = OTHER; note = 2'-O-methylguanosine
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyluridine
  modified_base 6: mod_base = OTHER; note = 2'-O-methyluridine
  modified_base 7: mod_base = OTHER; note = 2'-O-methyladenosine
  modified_base 8: mod_base = OTHER; note = 2'-fluorouridine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
aggattatctggatgtctaagcagccgaaaggctgc

SEQ ID NO: 61 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluorouridine phosphorothioate
  modified_base 3: mod_base = OTHER; note = 2'-fluoroadenosine
  modified_base 4: mod_base = OTHER; note = 2'-fluoroguanosine
  modified_base 5: mod_base = OTHER; note = 2'-fluoroadenosine
  modified_base 6: mod_base = OTHER; note = 2'-O-methylcytidine
  modified_base 7: mod_base = OTHER; note = 2'-fluoroadenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-fluorocytidine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylcytidine
  modified_base 20: mod_base = OTHER; note = 2'-O-methyluridine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
ttagacatccagataatcctgg

SEQ ID NO: 62 - DNA, 36 bp, synthetic construct
  source 1..36: mol_type = other DNA
  modified_base 1: mod_base = OTHER; note = 2'-O-methyladenosine phosphorothioate
  modified_base 2: mod_base = OTHER; note = 2'-O-methylcytidine
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-O-methylguanosine
  modified_base 8: mod_base = OTHER; note = 2'-fluoroadenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorouridine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
acaagagaagtcgtttcatagcagccgaaaggctgc

SEQ ID NO: 63 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluoroadenosine phosphorothioate
  modified_base 3: mod_base = OTHER; note = 2'-fluorouridine
  modified_base 4: mod_base = OTHER; note = 2'-fluoroguanosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-fluoroadenosine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methylguanosine
  modified_base 20: mod_base = OTHER; note = 2'-O-methyluridine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
tatgaaacgacttctcttgtgg

SEQ ID NO: 64 - DNA, 36 bp, synthetic construct
  source 1..36: mol_type = other DNA
  modified_base 1: mod_base = OTHER; note = 2'-O-methyladenosine phosphorothioate
  modified_base 2: mod_base = OTHER; note = 2'-O-methyladenosine
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-O-methyluridine
  modified_base 8: mod_base = OTHER; note = 2'-fluorouridine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methyluridine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA
aagtcgtttcattcaagttagcagccgaaaggctgc

SEQ ID NO: 65 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluoroadenosine phosphorothioate
  modified_base 3: mod_base = OTHER; note = 2'-fluoroadenosine
  modified_base 4: mod_base = OTHER; note = 2'-fluorocytidine
  modified_base 5: mod_base = OTHER; note = 2'-fluorouridine
  modified_base 6: mod_base = OTHER; note = 2'-O-methyluridine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-fluorouridine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methyluridine
  modified_base 20: mod_base = OTHER; note = 2'-O-methyluridine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
taacttgaatgaaacgacttgg

SEQ ID NO: 66 - DNA, 36 bp, synthetic construct; identical to SEQ ID NO: 33
  source 1..36: mol_type = other DNA
  modified_base 1: mod_base = OTHER; note = 2'-O-methylcytidine phosphorothioate
  modified_base 2: mod_base = OTHER; note = 2'-O-methyladenosine
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyluridine
  modified_base 8: mod_base = OTHER; note = 2'-fluorouridine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorouridine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylguanosine
  modified_base 22: mod_base = OTHER; note = 2'-O-methylcytidine
  modified_base 23: mod_base = OTHER; note = 2'-O-methyladenosine
  modified_base 24: mod_base = OTHER; note = 2'-O-methylguanosine
  modified_base 25: mod_base = OTHER; note = 2'-O-methylcytidine
  modified_base 26: mod_base = OTHER; note = 2'-O-methylcytidine
  modified_base 27: mod_base = OTHER; note = 2'-O-methylguanosine
  modified_base 28: mod_base = OTHER; note = 2'-O-GalNAc-modified adenosine
  modified_base 29: mod_base = OTHER; note = 2'-O-GalNAc-modified adenosine
  modified_base 30: mod_base = OTHER; note = 2'-O-GalNAc-modified adenosine
  modified_base 31: mod_base = OTHER; note = 2'-O-methylguanosine
  modified_base 32: mod_base = OTHER; note = 2'-O-methylguanosine
  modified_base 33: mod_base = OTHER; note = 2'-O-methylcytidine
  modified_base 34: mod_base = OTHER; note = 2'-O-methyluridine
  modified_base 35: mod_base = OTHER; note = 2'-O-methylguanosine
  modified_base 36: mod_base = OTHER; note = 2'-O-methylcytidine
  misc_feature 1..7: note = RNA
  misc_feature 8..11: note = DNA
  misc_feature 12..36: note = RNA

SEQ ID NO: 67 - DNA, 22 bp, synthetic construct
  source 1..22: mol_type = other DNA
  modified_base 1: mod_base = OTHER; note = 4'-O-monomethylphosphonate-2'-O-methyl uridine phosphorothioate
  modified_base 2: mod_base = OTHER; note = 2'-fluorouridine phosphorothioate
  modified_base 3: mod_base = OTHER; note = 2'-fluoroadenosine
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-fluoroadenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methyluridine
  modified_base 20: mod_base = OTHER; note = 2'-O-methylguanosine phosphorothioate
  modified_base 21: mod_base = OTHER; note = 2'-O-methylguanosine phosphorothioate
  modified_base 22: mod_base = OTHER; note = 2'-O-methylguanosine
  misc_feature 1: note = RNA
  misc_feature 2..3: note = DNA
  misc_feature 4: note = RNA
  misc_feature 5: note = DNA
  misc_feature 6: note = RNA
  misc_feature 7: note = DNA
  misc_feature 8..9: note = RNA
  misc_feature 10: note = DNA
  misc_feature 11..13: note = RNA
  misc_feature 14: note = DNA
  misc_feature 15..22: note = RNA
ttaaaattcaggaattcctggg

SEQ ID NO: 68 - skipped

SEQ ID NO: 69 - DNA, 15 bp, synthetic construct
  source 1..15: mol_type = other DNA
tgtgagagagatgct

SEQ ID NO: 70 - DNA, 24 bp, synthetic construct
  source 1..24: mol_type = other DNA
ccagcaacttcacaggagccaaaa

SEQ ID NO: 71 - skipped

SEQ ID NO: 72 - DNA, 13 bp, synthetic construct
  source 1..13: mol_type = other DNA
gggaagggactgg

SEQ ID NO: 73 - skipped

SEQ ID NO: 74 - DNA, 15 bp, synthetic construct
  source 1..15: mol_type = other DNA
gtcaaggtcgcaagc

SEQ ID NO: 75 - skipped

SEQ ID NO: 76 - DNA, 15 bp, synthetic construct
  source 1..15: mol_type = other DNA
acaaaggcccaaggc

SEQ ID NO: 77 - DNA, 26 bp, synthetic construct
  source 1..26: mol_type = other DNA
tggttaaggttgcaagcttgctggtg

SEQ ID NO: 78 - protein, 5 aa, synthetic construct
CREKA